(19) United States
(12) Patent Application Publication
(10) Pub. No.: US 2004/0053933 A1
(43) Pub. Date: Mar. 18, 2004

(54) Ligands of melanocortin receptors and compositions and methods related thereto

(75) Inventors: Joseph Pontillo, San Diego, CA (US); Dragan Marinkovic, Del Mar, CA (US); Marion C. Lanier, San Diego, CA (US); Joe Anh Tran, San Marcos, CA (US); Melissa Arellano, San Diego, CA (US); Jessica Parker, San Diego, CA (US); Jodie Nelson, San Diego, CA (US); Chen Chen, San Diego, CA (US); Fabio C. Tucci, San Diego, CA (US); Caroline Chen, San Diego, CA (US); Wanlong Jiang, San Diego, CA (US); Nicole White, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences, Inc., San Diego, CA

(21) Appl. No.: 10/434,803

(22) Filed: May 9, 2003

Related U.S. Application Data
(60) Provisional application No. 60/379,517, filed on May 10, 2002.
(60) Provisional application No. 60/422,272, filed on Oct. 29, 2002.

(51) Int. Cl. A61K 31/506; A61K 31/497; A61K 31/501; A61K 31/496; C 07D 4 3/02

(52) U.S. Cl. 514/252.02; 514/252.11; 514/252.14; 544/238; 544/295; 544/357; 544/360; 514/253.01

(57) ABSTRACT

Compounds which function as melanocortin receptor ligands and having utility in the treatment of melanocortin receptor-based disorders. The compounds have the following structure (I):

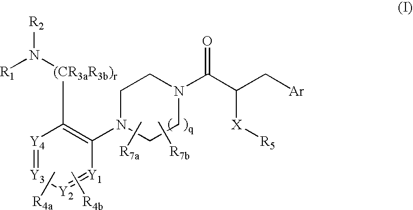

including stereoisomers, prodrugs, and pharmaceutically acceptable salts thereof, wherein Ar, R₁, R₂, R₃ₐ, R_{3b}, R₄ₐ, R_{4b}, R₅, R₇ₐ, R_{7b}, q, r, X, Y₁, Y₂, Y₃ and Y₄ are as defined herein. Pharmaceutical compositions containing a compound of structure (I), as well as methods relating to the use thereof, are also disclosed.

### CROSS-REFERENCE TO RELATED APPLICATIONS

This application claims the benefit of U.S. Provisional Application No. 60/379,517 filed May 10, 2002, and U.S. Provisional Application No. 60/422,272 filed Oct. 29, 2002 (both of which are hereby incorporated by reference in their entirety).

### BACKGROUND OF THE INVENTION

1. Field of the Invention

This invention is generally directed to ligands of a melanocortin receptor, as well as to compositions and methods for using such ligands to alter activity of a melanocortin receptor.

2. Description of the Related Art

Melanocortin (MC) receptors are members of the family of G-protein coupled receptors. To date, five distinct MC receptors (i.e., MC1-R, MC2-R, MC3-R, MC4-R and MC5-R) have been identified in a variety of tissues and these receptors have been shown to mediate a number of physiological processes. Ligands, including peptides and small molecules, have been shown to act as agonists or antagonists at these receptors.

The role of specific MC receptors in physiological processes has been the object of intense study since their discovery and cloning. These receptors are expressed in a variety of tissues including melanocytes, adrenal cortex, brain, gut, placenta, skeletal muscle, lung, spleen, thymus, bone marrow, pituitary, gonads and adipose tissue. A putative role of MC receptors has been shown in melanocytes, stimulatory actions on learning, attention and memory, motor effects, modification of sexual behavior, facilitation of nerve regeneration, anti-inflammatory and antipyretic effects, and the regulation of food intake and body weight.

The pro-opiomelanocortin (POMC) gene product is processed to produce a number of biologically active peptides that are expressed in the pituitary, and two locations in the brain: the arcuate nucleus of the hypothalamus and the solitary tract nucleus of the brain stem. These peptides elicit a range of biological activities. Two POMC peptides, α-melanocyte stimulating hormone (α-MSH) and adrenocorticotropic hormone (ACTH) control melanocyte and adrenocortical function, respectively, in the periphery.

Cloning studies have defined a family of five melanocortin (MC) receptors that respond to POMC peptides (reviewed in Rec. Prog. Hor. Res. 51:287-318, 1996). Each receptor in this family is pharmacologically distinct in its particular response to the POMC peptides α-MSH, γ-MSH and ACTH and to two peptide antagonists. Among the five receptors, MC4-R has the highest affinity for α-MSH. MC4-R differs from the other MC receptors in that it binds both natural melanocortin antagonists, agouti (Nature 371:799-802, 1994) and agouti-related protein (AgRP) (Biochem. Biophys. Res. Commun. 237:629-631, 1997). In contrast, MC1-R only binds agouti, MC2-R does not bind AgRP, MC3-R only binds AgRP, and MC5-R has only low affinity binding for AgRP (Mol. Endocrinology 13:148-155, 1999).

The expression of specific MC receptors is restricted anatomically. MC1-R is expressed primarily in melanocytes, while MC2-R is expressed in adrenocortical cells. MC3-R is expressed in brain, placenta and gut, and MC4-R is expressed primarily in the brain where its mRNA can be detected in nuclei that bind α-MSH. MC4-R is notably absent from adrenal cortex, melanocyte and placental tissues. Both MC3-R and MC4-R are expressed in arcuate and paraventricular neurons. MC5-R is expressed in brain, adipose tissues, muscle and exocrine glands.

α-Melanocyte stimulating hormone (α-MSH) is a tridecapeptide whose principal action (i.e., the activation of a set of G-protein coupled melanocortin receptors), results in a range of physiological responses including pigmentation, sebum production and feeding behavior. Cyclized peptide derivatives of α-MSH are potent modulators of these receptors. When administered by intracerebroventricular (i.c.v) injection into fasted animals, peptides exhibiting MCR-4 antagonist activity increase food intake and body weight. Moreover, overexpression of a naturally occurring peptide antagonist, agouti-related peptide (AgRP) has a similar effect on food intake and body weight. The development of small molecule antagonists of the MC4-R would selectively enhance the feeding response. MC4-R antagonists have a unique clinical potential because such compounds would stimulate appetite as well as decrease metabolic rate. Additionally, chronic MC4-R blockade causes an increase in lean body mass as well as fat mass, and the increase in lean body mass is independent of the increase in fat mass. Orally active forms of a small molecule MC4-R antagonist would provide a therapeutic strategy for indications in which cachexia is a symptom.

The MC receptors are also key mediators of steroid production in response to stress (MC2-R), regulation of weight homeostasis (MC4-R), and regulation of hair and skin pigmentation (MC1-R). They may have additional applications in controlling both insulin regulation (MC4-R) and regulation of exocrine gland function (MC5-R) (Cell 91:789-798, 1997); the latter having potential applications in the treatment of disorders such as acne, dry eye syndrome and blepharitis. Melanocortin peptides have also been reported to have anti-inflammatory activity, although the receptor(s) involved in mediating these effects have not yet been determined. Endocrine disorders such as Cushing's disease and congenital adrenal hyperplasia, which are characterized by elevated levels of ACTH, could be effectively treated with ACTH receptor (MC2-R) antagonists. Some evidence suggests that depression, which is characterized by elevated levels of glucocorticoids, may also be responsive to these same compounds. Similarly, elevated glucocorticoids can be an etiological factor in obesity. Synthetic melanocortin receptor agonists have been shown to initiate erections in men (J. Urol. 160:389-393, 1998). An appropriate MC receptor agonist could be an effective treatment for certain sexual disorders.

MC1-R provides an ideal target for developing drugs that alter skin pigmentation. MC1-R expression is localized to melanocytes where it regulates eumelanin pigment synthesis. Two small clinical trials indicate that broad-spectrum melanocortin agonists induce pigmentation with limited side effects. The desired compound would have a short half-life and be topically applied. Applications include skin cancer prevention, UV-free tanning, inhibition of tanning and treatment of pigmentation disorders, such as tyrosinase-positive albinism.

The role of melanocortin receptors in regulation of adiposity signaling and food intake has been recently reviewed (Nature 404:661-669,2000). Direct experimental evidence for the individual role of MC4 and MC3 receptors in energy homeostasis has not yet been reported due to the lack of potent and specific MC4 and MC3 agonists. Central administration of synthetic, non-selective MC3-R and MC4-R agonists, such as cyclic side-chain-lactam-modified peptide MT-II suppresses food intake in rodents and monkeys, and stimulates energy expenditure resulting in reduced adiposity (Endocrinology 142:2586-2592,2001). Conversely, selective peptide antagonists of the MC4 receptor stimulate food consumption and result in increased body weight, suggesting the main effects of agonist induced inhibition of food consumption are mediated by MC4 receptor activity. (European J Pharmacol. 405:25-32, 2000). Selective small molecule MC4-R antagonists also stimulate food intake in animal models of cachexia.

Genetically modified animals lacking the MC4 receptor are hyperphagic and obese (Cell 88:131-141, 1997). Humans with defective melanocortin 4 receptors exhibit marked hyperphagia and increased body mass relative to their normal siblings (Nature Genet. 20:111-114, 1998). In addition, studies with mice lacking functional MC3 receptors suggest that agonist stimulation of this receptor may also play a role in control of energy homeostasis, feeding efficiency, metabolism and bodyweight (Endocrinology 141:3518-3521, 2000). Therefore MC4-R and MC3-R agonists may be useful in the control of obesity and intreatment of related disorders including diabetes.

Accordingly, while significant advances have been made in this field, there is still a need in the art for ligands to the MC receptors and, more specifically, to agonists and/or antagonists to such receptors, particularly small molecules. There is also a need for pharmaceutical compositions containing the same, as well as methods relating to the use thereof to treat conditions associated with the MC receptors. The present invention fulfills these needs, and provides other related advantages.

### BRIEF SUMMARY OF THE INVENTION

In brief, this invention is directed to compounds that function as melanocortin (MC) receptor ligands. In this context, the term “ligand” means a molecule that binds, forms a complex with, or otherwise interacts with one or more of the MC receptors. This invention is also directed to compositions containing one or more of such compounds in combination with one or more pharmaceutically acceptable carriers, as well as to methods for treating conditions or disorders associated with MC receptors.

In one embodiment, this invention is directed to compounds that have the following structure (I):

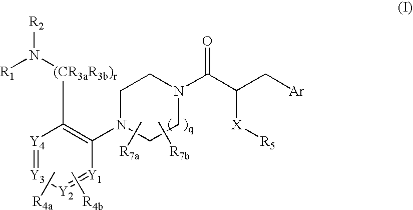

including stereoisomers, prodrugs, and pharmaceutically acceptable salts thereof, wherein Ar R₁, R₂, R₃ₐ, R_{3b}, R₄ₐ, R_{4b}, R₅, R₇ₐ, R_{7b}, q, r, X, Y₁, Y₂, Y₃ and Y₄ are as defined herein.

The compounds of this invention have utility over a broad range of therapeutic applications, and may be used to treat disorders or illnesses, including (but not limited to) eating disorders, obesity, inflammation, pain, skin disorders, skin and hair coloration, sexual dysfunction, dry eye, acne and/or Cushing's disease. A representative method of treating such a disorder or illness includes administering an effective amount of a compound of this invention, preferably in the form of a pharmaceutical composition, to an animal (also referred to herein as a “patient”, including a human) in need thereof. The compound may be an antagonist or agonist or may stimulate a specific melanocortin receptor while functionally blocking a different melanocortin receptor. Accordingly, in another embodiment, pharmaceutical compositions are disclosed containing one or more compounds of this invention in combination with a pharmaceutically acceptable carrier.

In one embodiment, the compounds of this invention are agonists to one or more MC receptors, and are useful in medical conditions where a melanocortin receptor agonist is beneficial. For example, the compounds of this invention may be utilized as MC4-R specific agonists or MC3-R specific agonists. Alternatively, the agonist may have mixed activity on the MC3 and MC4 receptor, and function as an antagonist of one of these receptors. In this context, the compounds of this invention may be used to treat obesity, erectile and/or sexual dysfunction, or diabetes mellitus.

In another embodiment, compounds of this invention may serve as antagonists to either the MC3-R or MC4-R receptor. Such antagonists have beneficial therapeutic effects, especially in the treatment of cachexia or wasting disease associated with cancer, AIDS, failure to thrive syndrome, and diseases associated with aging and senility. In more specific embodiments, the compounds are MC4-R antagonists for treatment of cachexia or wasting disease associated with cancer, AIDS, failure to thrive syndrome, and diseases associated with aging and senility.

These and other aspects of this invention will be apparent upon reference to the following detailed description and attached figures. To that end, certain patent and other documents are cited herein to more specifically set forth various aspects of this invention. Each of these documents is hereby incorporated by reference in its entirety.

### DETAILED DESCRIPTION OF THE INVENTION

As mentioned above, in one embodiment the present invention is generally directed to compounds having the following structure (I):

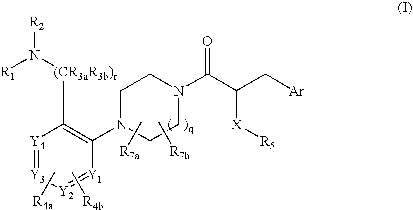

or a stereoisomer, prodrug or pharmaceutically acceptable salt thereof,

wherein:

q is 1 or2;

r is 1,2, or 3;

Y₁, Y₂, Y₃ and Y₄ are independently CH or N, with the proviso that no more than two of Y₁, Y₂, Y₃ and Y₄ are N, and with the further proviso that, when two of Y₁, Y₂, Y₃ and Y₄ are N, either Y₁ and Y₃ are N or Y₂ and Y₄ are N;

Ar is phenyl, substituted phenyl, naphthyl, or substituted naphthyl;

X is a bond, —O—, —S—, —N(R₆ₐ)—, —N(R₆ₐ)C(═O)—, —N(R₆ₐ)S(═O)₂—, —N(R₆ₐ)C(═O)NR_{6b}—, —C(═O)O—, —OC(═O)—, —N(R₆ₐ)C(═O)NR_{6b}O—, N(R₆ₐ)C(═O)NNR_{6c}—, or —N(R₆ₐ)C(═O)O—;

R₁ and R₂ are the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;

R₃ₐ and R_{3b} are, at each occurrence, the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;

R₄ₐ and R_{4b} are optional ring substituents and, when one or both are present, are the same or different and independently hydroxy, alkyl, substituted alkyl, cyano, halogen, alkoxy, or alkylamino;

R₅ is hydrogen, alkyl, substituted alkyl, aryl, substituted aryl, heterocycle, or substituted heterocycle;

R₆ₐ, R_{6b} and R_{6c} are, at each occurrence, the same or different and independently hydrogen, alkyl, or substituted alkyl; and

R₇ₐ and R_{7b} are optional ring substituents and, when one or both are present, are the same or different and independently hydrogen, lower alkyl, or substituted lower alkyl;

with the proviso that when r is 1 then R₁, R₂, R₃ₐ and R_{3b} are not all hydrogen.

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂cyclohexyl, and the like; while unsaturated cyclic alkyls include cyclopentenyl, cyclohexenyl, —CH₂cyclohexenyl, and the like. Cyclic alkyls are also referred to herein as a “homocycle” or “homocyclic ring”, including bicyclic rings in which the homocycle is fused to a benzene ring. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Aryl” means an aromatic carbocyclic moiety such as phenyl or naphthyl.

“Arylalkyl” means an alkyl having at least one alkyl hydrogen atom replaced with an aryl moiety, such as benzyl (i.e., —CH₂phenyl), —(CH₂)₂phenyl, —(CH₂)₃phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, oxadiazolyl, benzoxadiazolyl, thiadiazolyl, indazolyl and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” (also referred to herein as a “heterocyclic ring”) means a 4- to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, piperazinyl, piperazinonyl, piperazindionyl, pyrrolidindionyl, azetidinyl, azetidinonyl, oxetanonyl, thietanyl, thietanonyl, thietanedionyl, thietanetrionyl, tetrahydrofuranonyl, tetrahydrothiophenyl S-oxide, tetrahydrothiophenyl S-dioxide, pyridinonyl, piperidinonyl, homopiperidinyl, homopiperidinonyl, imidazolinyl, imidazolonyl, pyrazolinyl, pyrazolinonyl, oxazolinyl, oxazolinonyl, isooxazolinyl, isooxazolinonyl, thiazolinyl, thiazolinonyl, isothiazolyl, isothiazolinyl, isothiazolinonyl, morpholinonyl, 1,4-thiazinanyl, 1,4-thiazinanonyl, 1,4-thiazinane-dionyl, 1,4-thiazinane-trionyl, pyrimidinonyl, tetrahydro-1,3-diazinonyl, tetrahydro-1,3-oxazinonyl, tetrahydro-1,3-thiazinonyl, hexahydropyridazinyl, tetrahydropyridazinonyl, tetrahydro-1,2-oxazinyl, tetrahydro-1,2-oxazinonyl, 1,2-thiazinane-dionyl, 1,2-thiazinane-trionyl, 1,2-diazepinyl, 1,2-diazepinonyl, 1,2-oxazepinyl, 1,2-oxazepinonyl, 1,2-thiazepinyl, 1,2-thiazepinonyl, 1,3-diazepinyl, 1,3-diazepinonyl, 1,3-oxazepinyl, 1,3-oxazepinonyl, 1,3-thiazepinyl, 1,3-thiazepinonyl, homopiperazinyl, homopiperazinonyl, homomorpholinyl, homomorpholinonyl, homothiazepine, homothiazepinonyl, homothiazepinedionyl, homothiazepinetrionyl, and the like.

“Heterocyclealkyl” means an alkyl having at least one alkyl hydrogen atom replaced with a heterocycle, such as —CH₂morpholinyl, and the like.

The term “substituted” as used herein means any of the above groups (i. e., alkyl, aryl, arylalkyl, heteroaryl, heteroarylalkyl, heterocycle and heterocyclealkyl) wherein at least one hydrogen atom is replaced with a substituent. In the case of a oxo substituent (“═O”) two hydrogen atoms are replaced. When substituted, “substituents” within the context of this invention include oxo, halogen, hydroxy, cyano, nitro, amino, alkylamino, dialkylamino, alkyl, alkoxy, thioalkyl, sulfonylalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐNR_{b}, —NRₐC(═O)OR_{b} —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ, —S(═O)₂ORₐ, —CH₂S(═O)₂Rₐ, —CH₂S(═O)₂N(Rₐ)₂, ═NS(═O)₂Rₐ, and —S(═O)₂N(Rₐ)₂, wherein Rₐ and R_{b} are the same or different and independently hydrogen, alkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

“Halogen” means fluoro, chloro, bromo and iodo.

“Haloalkyl” means an alkyl having at least one hydrogen atom replaced with halogen, such as trifluoromethyl and the like.

“Alkoxy” means an alkyl moiety attached through an oxygen bridge (i.e., —O-alkyl) such as methoxy, ethoxy, and the like.

“Thioalkyl” means an alkyl moiety attached through a sulfur bridge (i.e., —S-alkyl) such as methylthio, ethylthio, and the like.

“Sulfonylalkyl” means an alkyl moiety attached through a sulfonyl bridge (i.e., —S0₂-alkyl) such as methylsulfonyl, ethylsulfonyl, and the like.

“Alkylamino” and “dialkylamino” mean one or two alkyl moieties, respectively, attached through a nitrogen bridge (i.e., —N-alkyl) such as methylamino, ethylamino, dimethylamino, diethylamino, and the like.

“Hydroxyalkyl” means an alkyl substituted with at least one hydroxyl group.

In addition, it should be understood that each and everyone combination of above groups—that is, q, r, Y₁, Y₂, Y₃, Y₄, Ar, X, R₁, R₂, R₃ₐ, R_{3b}, R₄ₐ, R_{4b}, R₅, R₆ₐ, R_{6b}, R₇ₐ and R_{7b} (with the exception of those specific embodiment removed by negative proviso)—are specifically disclosed within and encompassed by this invention.

In one embodiment, compounds of this invention have structure (II) when q is 1 and structure (III) when q is 2:

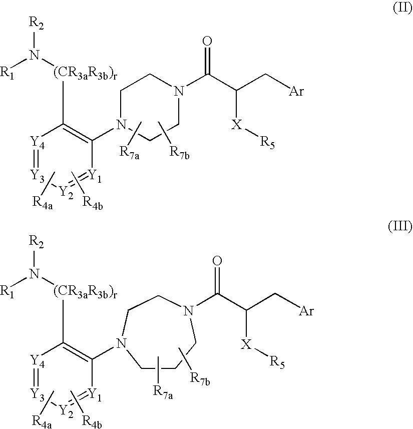

In another embodiment, compounds of this invention have structure (IV) when each of Y₁, Y₂, Y₃ and Y₄ are CH:

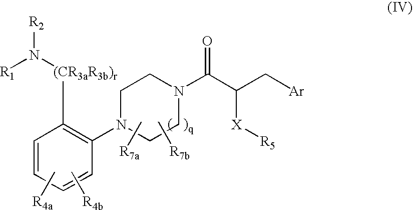

In another embodiment, compounds of this invention have structure (V), (VI) (VII) or (VIII) when one of Y₁, Y₂, Y₃ and Y₄ are N (the remainder being CH):

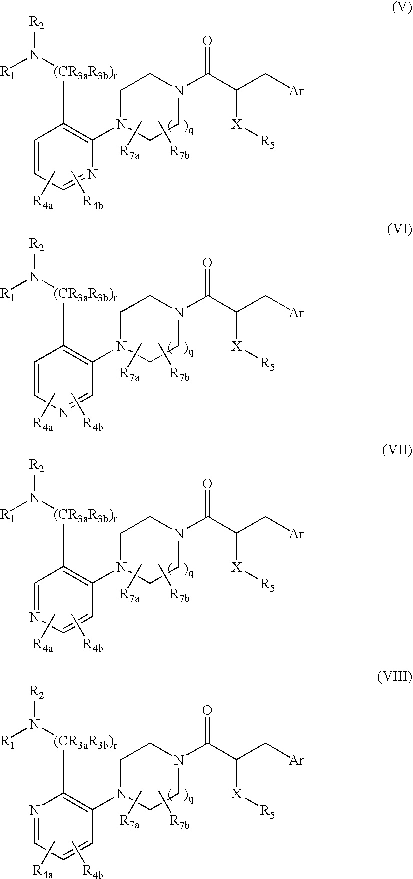

In another embodiment, compounds of this invention have structures (IX) or (X) when two of Y₁, Y₂, Y₃ and Y₄ are N (the remainder being CH):

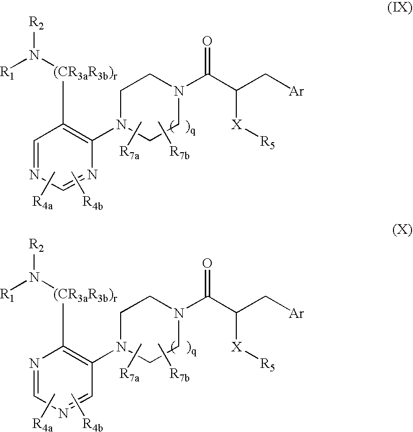

In a further embodiment, X is an amide bond (“—N(R₆ₐ)C(═O)—”) and compounds of this invention have structure (XI), while in still a further embodiment Ar is phenyl substituted with, for example, halogen as represented by structure (XII):

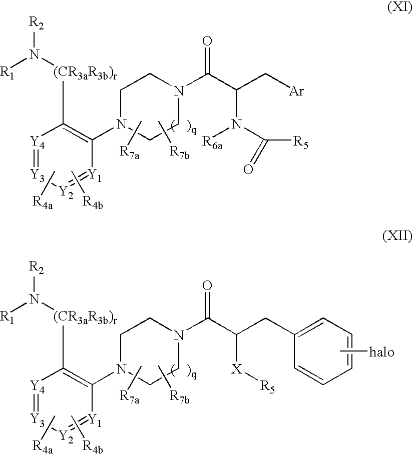

In still further embodiments, the compounds of this invention have the following structure (XIII) when r is 1 and structure (XIV) with r is 2:

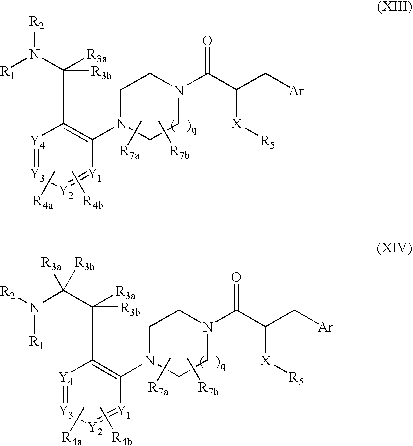

In yet another embodiment, R₁ and/or R₂ are not joined to the nitrogen atom via an amide bond—that is, R₁ and/or R₂ are not joined to the nitrogen atom through a carbonyl which, when taken together with the nitrogen atom, would form a “C(═O)N” linkage. Such a linkage could be formed if one or both of R₁ and R₂ were substituted alkyl, wherein the carbon atom joined to the nitrogen atom was substituted with oxo (i.e., ═O). Thus, in this embodiment, compounds of structure (I) do not include compounds having the following structures:

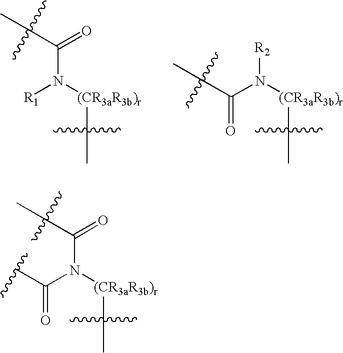

wherein “ [symbol] ” represents the remainder of structure (I).

In a further embodiment of this invention, X is —N(R₆ₐ)— where R₆ₐ is alkyl or substituted alkyl, as represented by compounds having structures (XV) and (XVI):

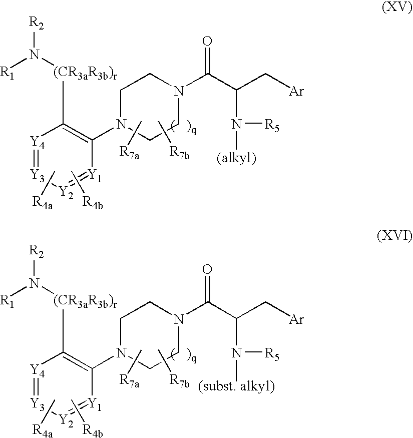

In still a further embodiment, X is a bond, and compounds of this invention have the following structure (XVII):

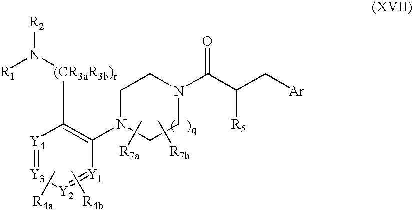

In a more specific embodiment of structure (XVII), R₅ is a heterocycle or substituted heterocycle, as represented by compounds having structures (XVIII) and (XIX):

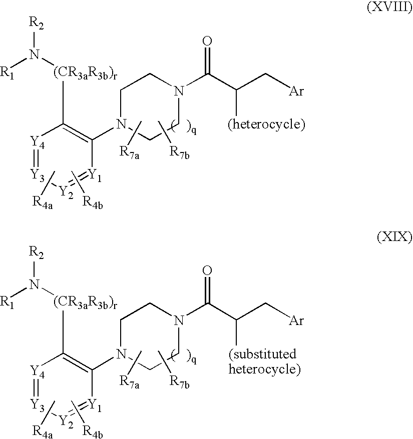

In another more specific embodiment of structure (XVII), R₅ is hydrogen, and compounds of this invention have structure (XX):

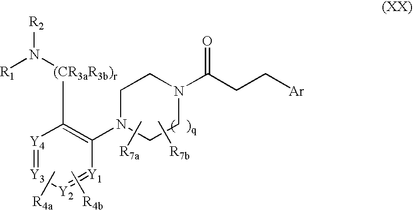

An another embodiment, X is —S—, —N(R₆ₐ)—, —N(R₆ₐ)C(═O)—, —N(R₆ₐ)S(═O)₂—, —N(R₆ₐ)C(═O)NR_{6b}—, —C(═O)O—, or —N(R₆ₐ)C(═O)O—, wherein R₆ₐ is alkyl or substituted alkyl as represented by the following structures (XXI) through (XVII):

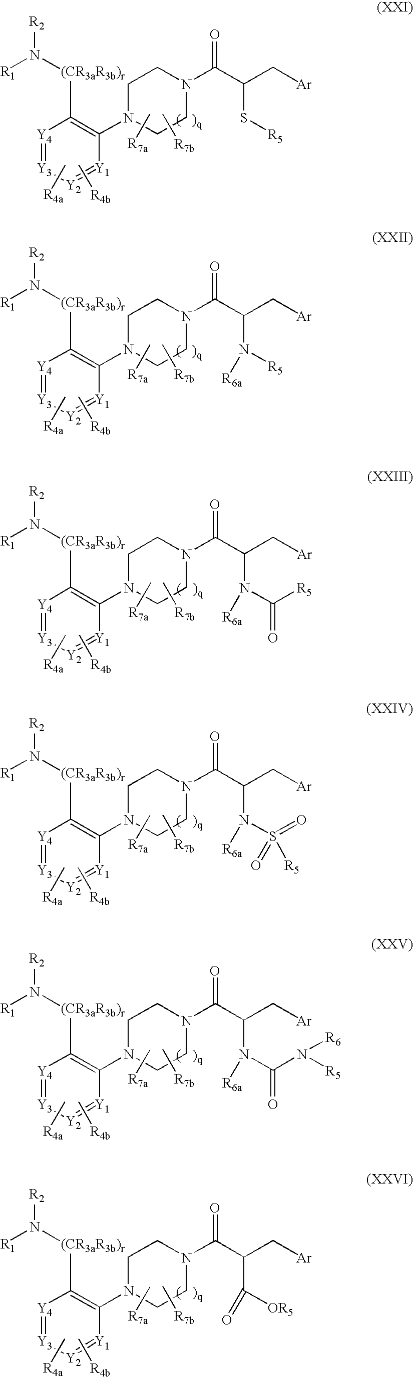

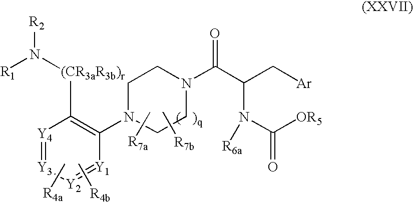

The compounds of the present invention may be prepared by known organic synthesis techniques, including the methods described in more detail in the following Reaction Schemes and Examples (at some instances, NH is simply shown as N for purpose of abbreviation). Furthermore, compounds of the present invention may be synthesized by a number of methods, both convergent and sequential, utilizing solution or solid phase chemistry.

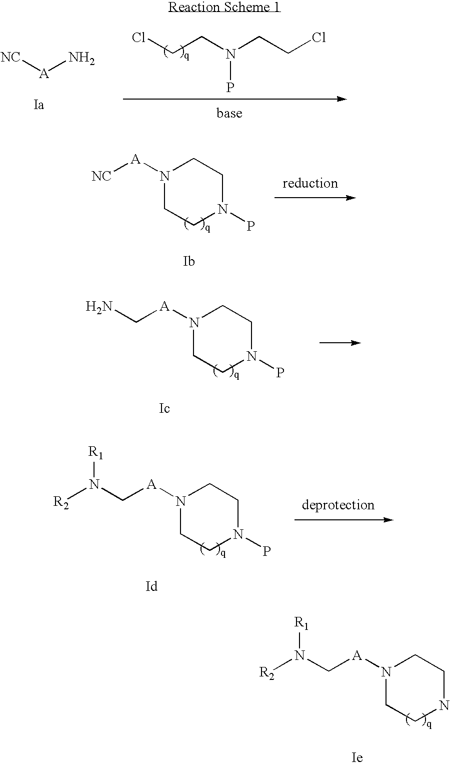

An aromatic group “A” (i.e., phenyl, pyridyl or pyrimidinyl optionally substituted with one or both of R₄ₐ and R_{4b}) directly substituted with a cyano and a NH₂ group, illustrated as 1a, may be reacted with a protected bis (2-chloroethyl)amine under basic conditions to produce 1b. Reduction of 1b produces intermediate 1c that can further react in various ways to form a large number of secondary or tertiary amines 1d. Reagents used to obtain 1d can be aldehydes, ketones, alkyl and aryl halides but are not limited to these. When the reagent is a keto compound, reductive amination of 1c using a reducing agent such as sodium triacetoxyborohydride in solvent such as dichloroethane in the presence or not of an acid catalyst such as acetic acid at 0 to 100° C. for 1-24 hours gives 1d. Halides addition can be used in basic conditions such as triethylamine to get to 1d. A combination of halide addition and/or reductive amination can also be used. 1d was then deprotected to give 1e.

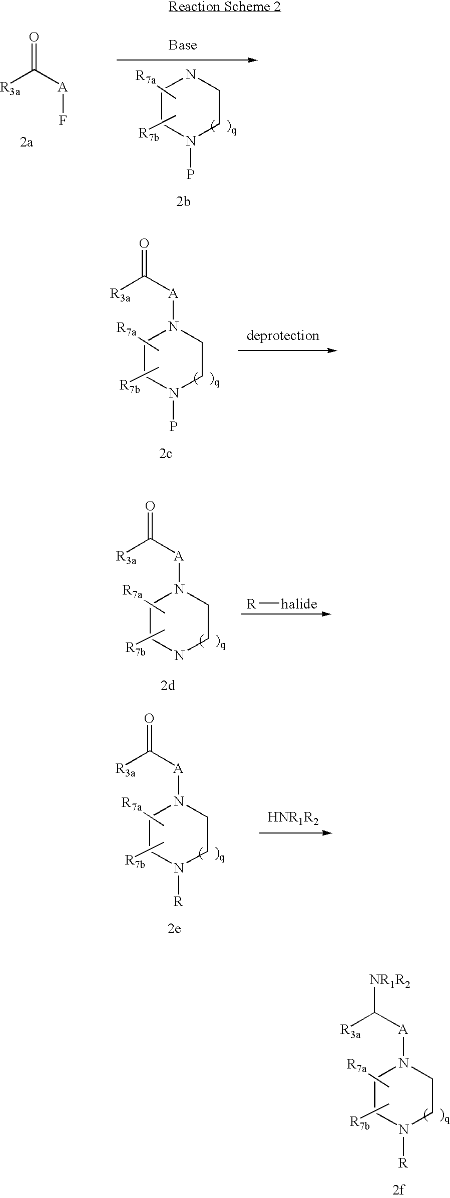

An aromatic group A directly substituted by halogen such as fluorine and a ketone, illustrated as 2a, can be reacted with 2b in basic conditions such as potassium carbonate in solvent such as DMSO or dimethylformamide, at 25 to 150° C. for 1-24 hours to yield 2c. 2c is then deprotected to give 2d and mixed with various R-halide to give 2e. Reductive amination of 2e with an appropriate amine using a reducing agent such as sodium triacetoxyborohydride in solvent such as dichloroethane in the presence or not of an acid catalyst such as acetic acid at 0 to 100° C. for 1-24 hours gives 2f.

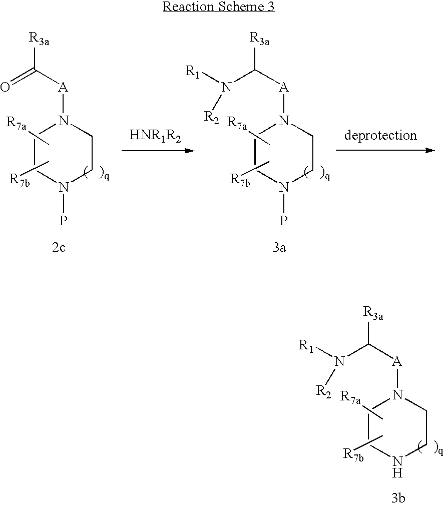

Reductive amination of 2c with an appropriate amine using a reducing agent such as sodium triacetoxyborohydride in solvent such as dichloroethane in the presence or not of an acid catalyst such as acetic acid at 0 to 100° C. for 1-24 hours gives 3a. When R₁ and/or R₂ is a hydrogen, 3a can be further reacted either with an alkyl or aryl halide or undergo reductive amination. 3a can be deprotected to give 3b.

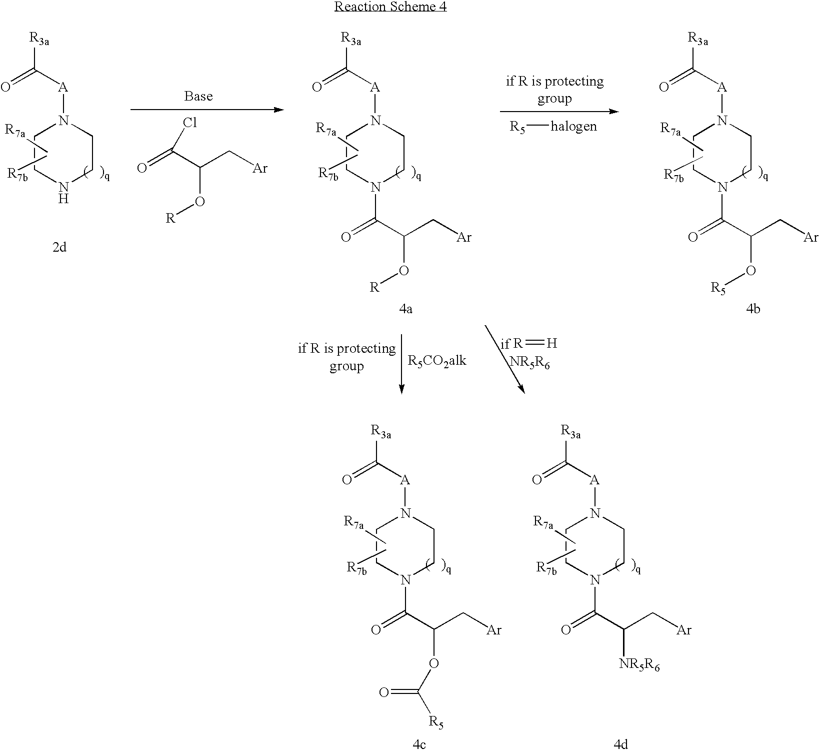

To compound 2d is added an acid halide in presence of base such as triethylamine to give 4a. When R is an alcohol protecting group, 4a can further react with an electrophile. The ether derivative 4b can be prepared by treatment of deprotected 4a with an alkyl halide and a base such as potassium carbonate or sodium hydroxide in an inert organic solvent such as acetone, dimethylformamide or DMSO at a temperature of 25 to 100° C. for a period of 1-72 hours. Deprotected 4a can also be reacted with an ester such as alkyl ester R₅COO(alkyl) to give 4c. Treatment of 4a with mesyl or tosyl chloride in methylene chloride with a base such as triethylamine or pyridine at 0 to 100° C. for 1-24 hours followed by reaction with an amine in a solvent such as DMF or toluene for 0.5-12 hours at 25 to 100° C. gives 4d.

The same synthetic route may be followed substituting compound 1e or 3b for compound 2d in the above procedure.

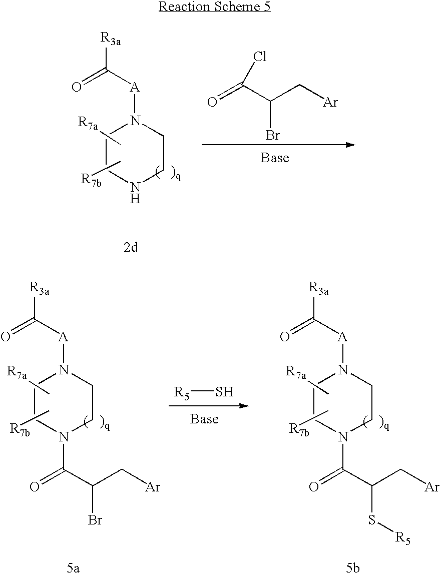

To compound 2d is added 2-bromo ethanoyl chloride in presence of base such as triethylamine to give 5a. 5a is reacted with a nucleophile such as a thiol to give 5b.

The same synthetic route may be followed substituting compound 1e or 3b for compound 2d in the above procedure.

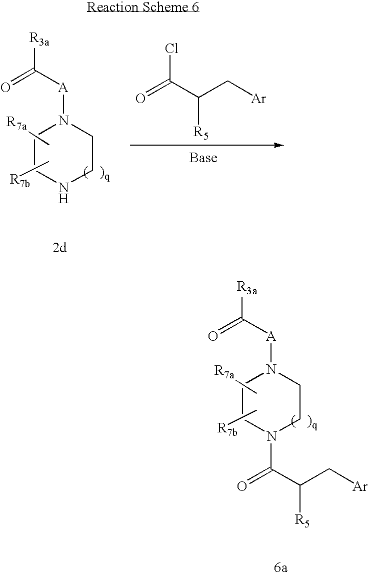

To compound 2d is added an acid chloride in presence of base such as triethylamine in inert solvent such as methylene chloride to give 6a.

The same synthetic route may be followed substituting compound 1e or 3b for compound 2d in the above procedure.

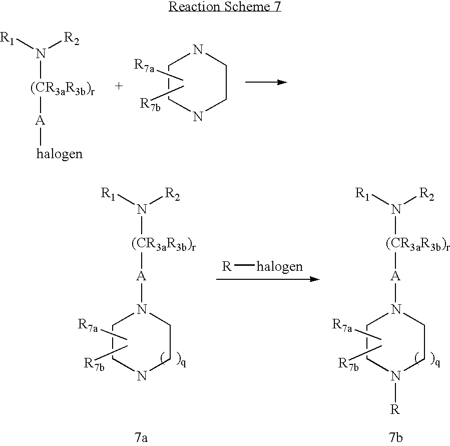

Piperazine or protected piperazine may be alkylated with an appropriate halogenated compound to give compound 7a which may be reacted with the various reagents as used in reaction schemes 4, 5, 6 to give compound 7b.

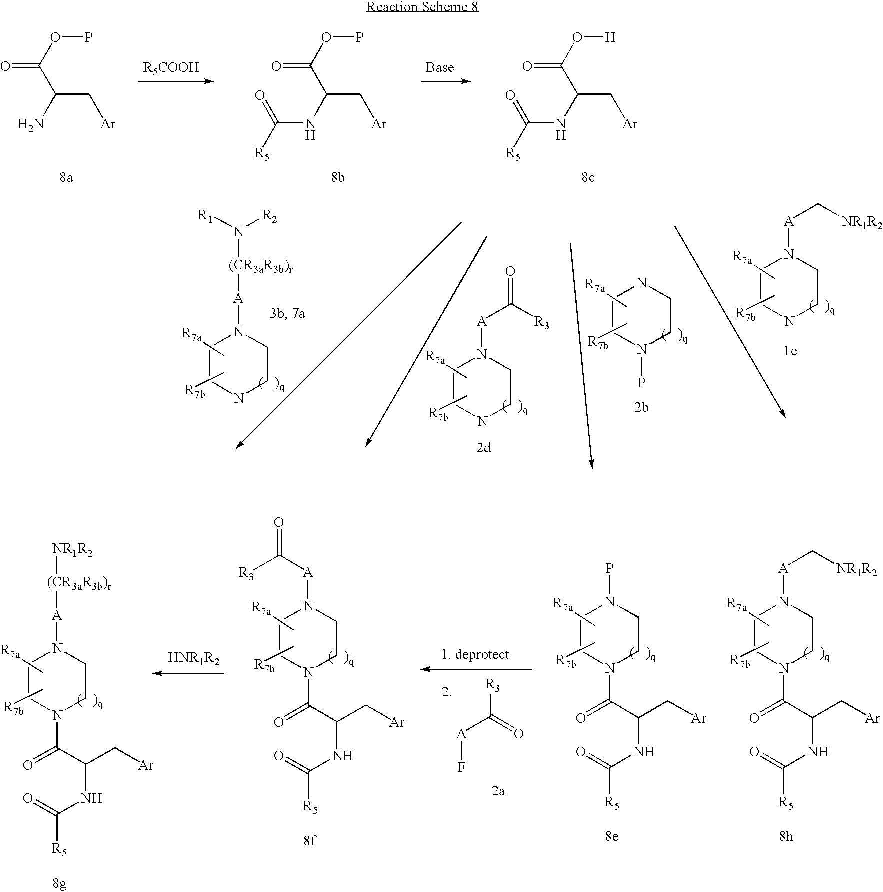

Compound 8a reacted with an acid under standard coupling conditions gives 8b. 8b is saponified in presence of a base such as LiOH or NaOH to give 8e. 8c is then coupled to 2b using standard peptide coupling procedures to give 8e. Product 8e is then deprotected and reacted with 2a under basic conditions such as potassium carbonate in a solvent such as DMSO or dimethylformamide at 25 to 150° C. for 1-24 hours to yield 8f. Reductive amination of 8f with an appropriate amine using a reducing agent such as sodium triacetoxyborohydride in solvent such as dichloroethane in the presence or not of an acid catalyst such as acetic acid at 0 to 100° C. for 1-24 hours gives 8g.

8c is similarly coupled to 2d, 3b or 7a, and 1e to give 8f, 8g, and 8h, respectively, using standard peptide coupling procedures.

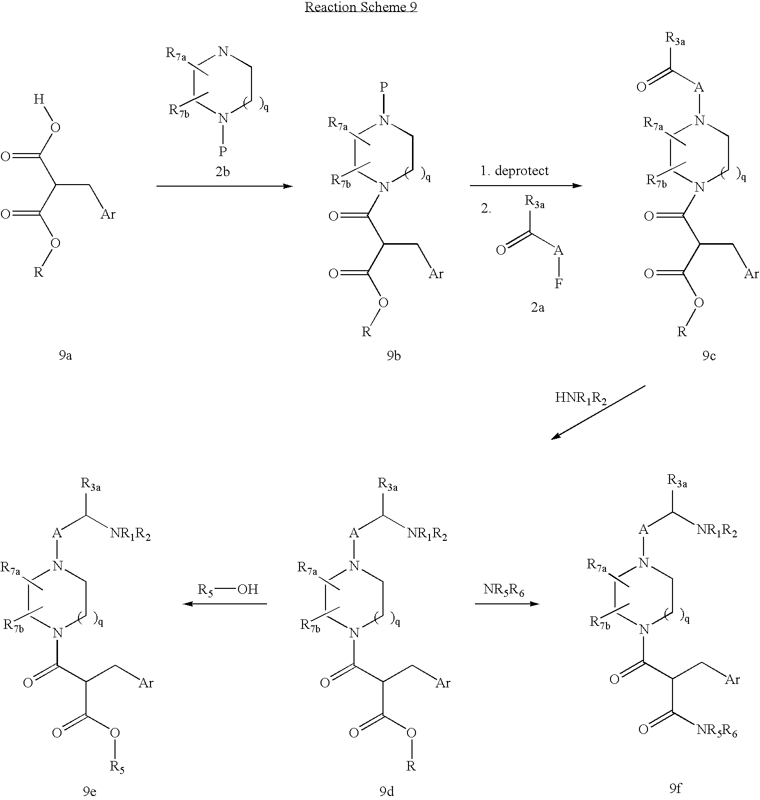

Compound 9a is reacted with 2b using conventional peptide coupling methods to yield compound 9b. 9b is then deprotected and reacted with compound 2a in basic conditions such as potassium carbonate in a solvent such as DMSO or dimethylformamide at 25 to 150° C. for 1-24 hours to yield to 9c. Reductive amination of 9c with an appropriate amine using a reducing agent such as sodium triacetoxyborohydride in solvent such as dichloroethane optionally in the presence of an acid catalyst such as acetic acid at 0 to 100° C. for 1-24 hours gives 9d. Ester 9d can subsequently be transesterified with an alcohol R₅—OH or reacted with a substituted amine HNR₁R₂ and a Lewis acid such as triethylaluminium in a solvent such as chloroform or benzene to give the amide 9f after 1-24hours at 0 to 100° C.

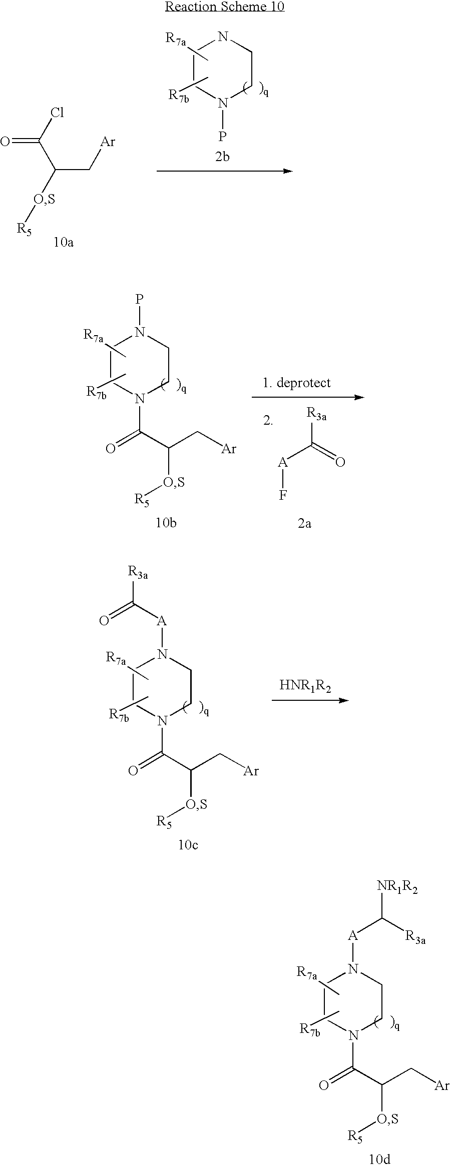

Compound 10a is reacted in basic conditions such as triethylamine with 2b to give the amide compound 10b. 10b is then deprotected and reaction with 2a in basic conditions such as potassium carbonate in a solvent such as DMSO or dimethylformamide at 25 to 150° C. for 1-24 hours yields 10c. Reductive amination of 10c with an appropriate amine using a reducing agent such as sodium triacetoxyborohydride in solvent such as dichloroethane optionally in the presence of an acid catalyst such as acetic acid at 0 to 100° C. for 1-24 hours gives 10d.

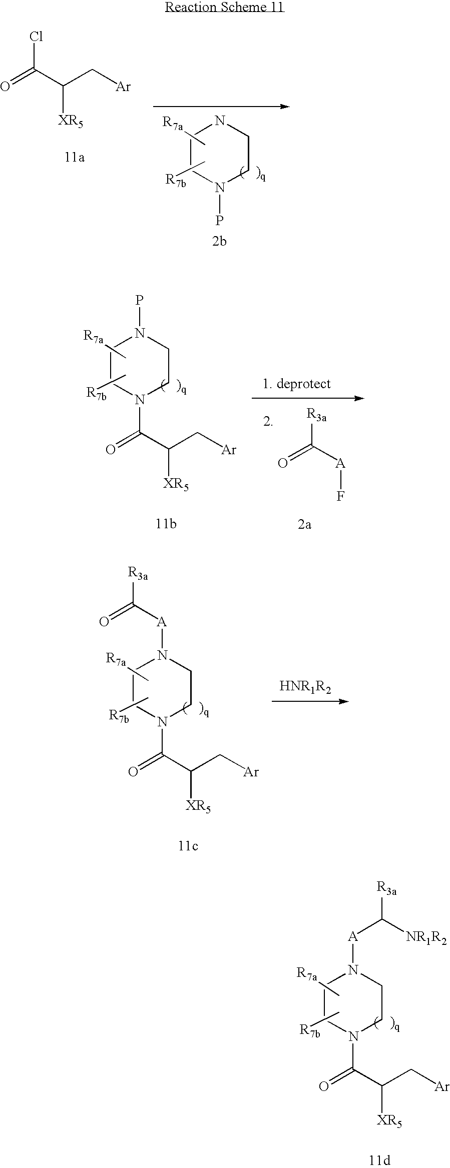

Compound 11a is reacted in basic conditions such as triethylamine with 2b to give the amide compound 11b. 11b is then deprotected and reaction with 2a in basic conditions such as potassium carbonate in a solvent such as DMSO or dimethylformamide at 25 to 150° C. for 1-24 hours yields 11c. Reductive amination of 11c with an appropriate amine using a reducing agent such as sodium triacetoxyborohydride in a solvent such as dichloroethane optionally in the presence of an acid catalyst such as acetic acid at 0 to 100° C. for 1-24 hours gives 11d.

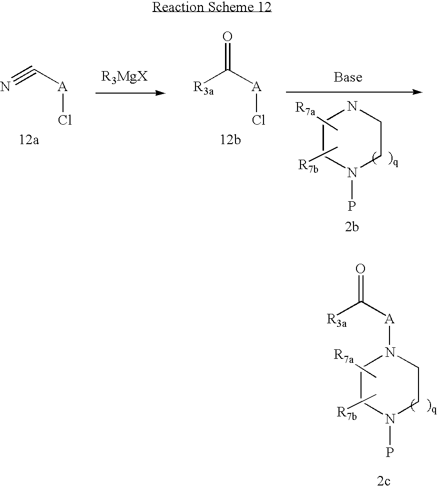

An aromatic group A directly substituted by a cyano group and a halogen such as chlorine, 12a, can undergo a Grignard reaction using standard conditions with R₃MgX such as methyl magnesium iodide to give 12b. 12b can then react with 2b in basic conditions such as potassium carbonate in solvent such as DMSO or dimethylformamide at 25 to 150° C. for 1-24 hours to yield to 2c.

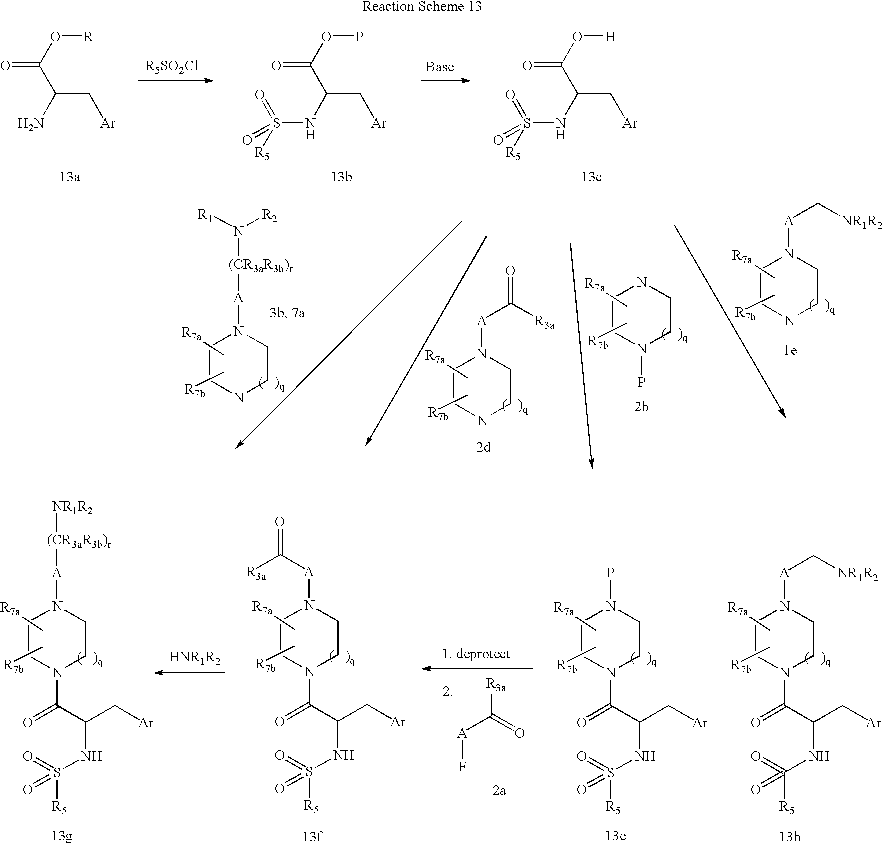

Ester 13a is reacted with a sulfonyl chloride in basic medium to give 13b. 13b is saponified in presence of base such LiOH or NaOH to give 13c. 13c is then coupled to 2b using standard peptide coupling procedures. Product 13e is then deprotected and reacted with 2a under basic conditions such as potassium carbonate in solvent such as DMSO or dimethylformamide at 25 to 150° C. for 1-24 hours to yield to 13f. Reductive amination of 13f with an appropriate amine using a reducing agent such as sodium triacetoxyborohydride in solvent such as dichloroethane optionally in the presence of an acid catalyst such as acetic acid at 0 to 100° C. for 1-24 hours gives 13g. 13c is similarly coupled to 2d, 3b and 1e to give 13f, 13g and 13h respectively using standard peptide coupling procedures.

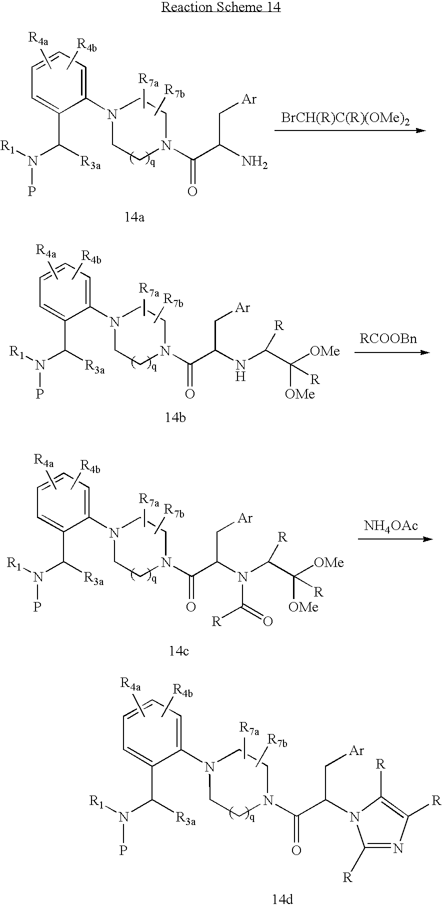

Protected amine 14a (e.g., where P is Boc) is alkylated with an appropriate compound such as an alkyl halide. In the current scheme the reagent is a substituted bromoketal which gives compound 14b. Addition of a protected carboxylic acid gives 14c. Cyclization with an appropriate reagent such as ammonium acetate gives substituted or unsubstituted imidazole compound 14d, which may be deprotected under acidic conditions. In this reaction scheme, as well as the following reaction schemes, R is at each occurrence the same or different and represents a substituent as defined above.

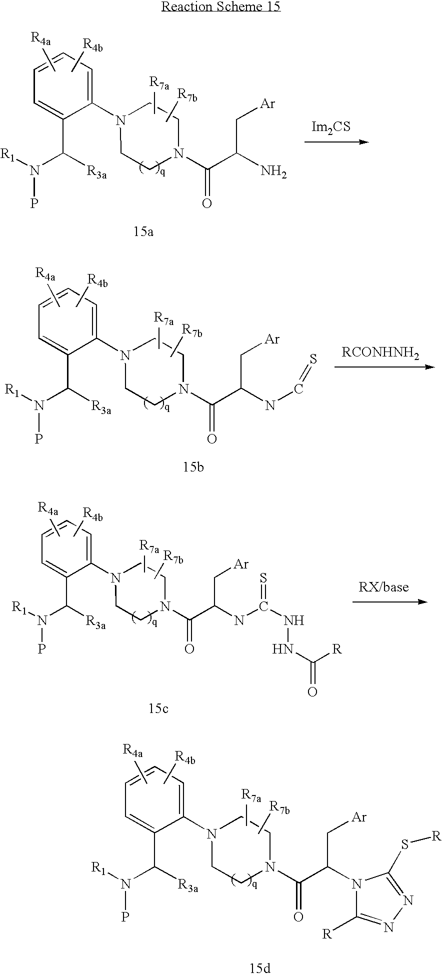

Protected amine 15a and thiocarbonyl diimidazole gives the thioisocyanate 15b. Reaction with an appropriate hydrazide gives compound 15c. 15c and alkyl halide in the presence of a base gives the substituted triazole 15d, which may be deprotected under acidic conditions.

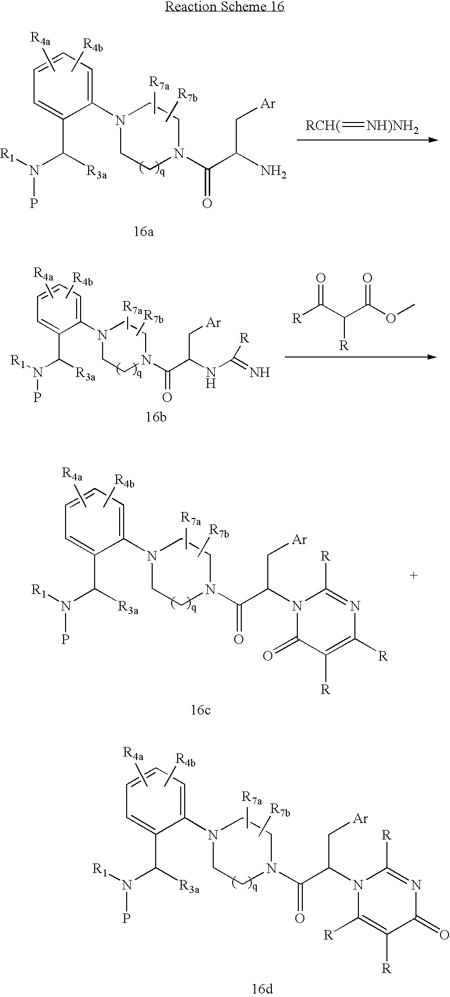

Protected amine 16a and a amidine give compound 16b. Reation with an acetoacetate gives cyclized products 16c and 16d. The Boc group may be deprotected under acidic conditions.

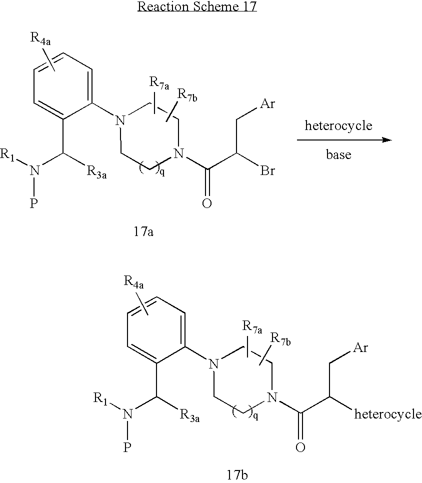

Bromo compound 17a and an appropriate heterocycle (including substituted heterocycle) or amine containing compound forms compound 17b in the presense of a base. Treatment with trifluoroacetic acid in methylene chloride or HCl in methylene chloride removes the Boc protecting group.

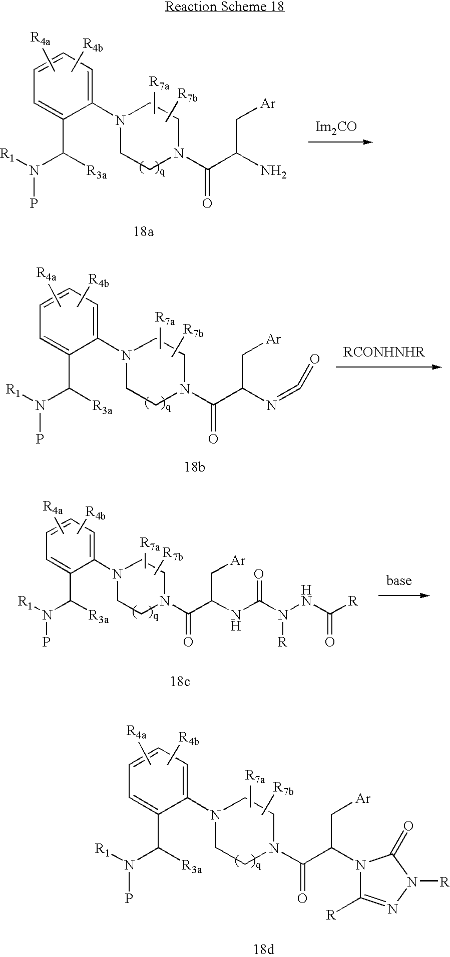

Protected amine 18a and carbonyl diimidazole gives the isocyanate 18b. Reaction with a hydrazide gives compound 18c which cyclizes under basic conditions to give 18d which may be deprotected under acidic conditions.

Representative compounds of this invention include the following:

1-[2R-acetamido-3-(2,4-dichlorophenyl)propionyl]-4-[2-(N-methyl-2-methoxyethyl)aminomethylphenyl]piperazine;

1-[2R-(2-aminoacetamido)-3-(2,4-dichlorophenyl)propionyl]-4-[2-(N-methyl-2-methoxyethyl)aminomethylphenyl]piperazine;

1-[2R-(3-aminopropionamido)-3-(2,4-dichlorophenyl)propionyl]-4-[2-(N-methyl-2-methoxyethyl)aminomethylphenyl]piperazine;

1-[2R-acetamido-3-(2,4-dichlorophenyl)propionyl]-4-[2-(1R-methyl-2-methoxyethyl)aminomethylphenyl]piperazine;

1-[2R-acetamido-3-(2,4-dichlorophenyl)propionyl]-4-[2-(1S-methyl-2-methoxyethyl)aminomethylphenyl]piperazine;

1-[2R-(2-aminoacetamido)-3-(2,4-dichlorophenyl)propionyl]-4-[2-(1-methyl-2-methoxyethyl)aminomethylphenyl]piperazine;

1-[2R-(2-methylaminoacetamido)-3-(2,4-dichlorophenyl)propionyl]-4-[2-(1-methyl-2-methoxyethyl)aminomethylphenyl]piperazine;

1-[2R-(2-dimethylaminoacetamido)-3-(2,4-dichlorophenyl)propionyl]-4-[2-(1-methyl-2-methoxyethyl)aminomethylphenyl]piperazine;

1-[2R-(3-aminopropionamido)-3-(2,4-dichlorophenyl)propionyl]-4-[2-(1-methyl-2-methoxyethyl)aminomethylphenyl]piperazine;

1-[2R-(3-methylaminopropionamido)-3-(2,4-dichlorophenyl)propionyl]-4-[2-(1-methyl-2-methoxyethyl)aminomethylphenyl]piperazine;

1-[2R-(3-dimethylaminopropionamido)-3-(2,4-dichlorophenyl)propionyl]-4-[2-(1-methyl-2-methoxyethyl)aminomethylphenyl]piperazine;

1-[2R-(2-methylaminoacetamido)-3-(2,4-dichlorophenyl)propionyl]-4-[2-(1-methoxyethylamino)ethylphenyl]piperazine;

1-[2R-(2-dimethylaminoacetamido)-3-(2,4-dichlorophenyl)propionyl]-4-[2-(1-amino-3-methyl)butylphenyl]piperazine;

1-[2R-(2-methylaminoacetamido)-3-(2,4-dichlorophenyl)propionyl]-4-[2-(1-methyl-2-methoxyethyl)aminomethyl-4-(trifluoromethyl)phenyl]piperazine;

1-[2R-(2-dimethylaminoacetamido)-3-(2,4-dichlorophenyl)propionyl]-4-[2-(1-methyl-2-methoxyethyl)aminomethyl-4-(trifluoromethyl)phenyl]piperazine;

1-[2R-(1-piperazinylcarboxamido)-3-(2,4-dichlorophenyl)propionyl]-4-[2-(1-methyl-2-methoxyethyl)aminomethyl-4-(trifluoromethyl)phenyl]piperazine;

1-[2R-(4-methyl-1-piperazinylcarboxamido)-3-(2,4-dichlorophenyl)propionyl]-4-[2-(1-methyl-2-methoxyethyl)aminomethyl-4-(trifluoromethyl)phenyl]piperazine;

1-[2R-(4-ethyl-1-piperazinylcarboxamido)-3-(2,4-dichlorophenyl)propionyl]-4-[2-(1-methyl-2-methoxyethyl)aminomethyl-4-(trifluoromethyl)phenyl]piperazine;

1-[2R-(4-isopropyl-1-piperazinylcarboxamido)-3-(2,4-dichlorophenyl)propionyl]-4-[2-( 1-methyl-2-methoxyethyl)aminomethyl-4-(trifluoromethyl)phenyl]piperazine;

1-[2R-(4-benzyl-1-piperazinylcarboxamido)-3-(2,4-dichlorophenyl)propionyl]-4-[2-(1-methyl-2-methoxyethyl)aminomethyl-4-(trifluoromethyl)phenyl]piperazine;

1-{2R-[4-(2-pyridyl)-1-piperazinylcarboxamido]-3-(2,4-dichlorophenyl)propionyl}-4-[2-(1-methyl-2-methoxyethyl)aminomethyl-4-(trifluoromethyl)phenylpiperazine;

1-{2R-[4-(2-pyrimidyl)-1-piperazinylcarboxamido]-3-(2,4-dichlorophenyl)propionyl}-4-[2-(1-methyl-2-methoxyethyl)aminomethyl-4-(trifluoromethyl)phenyl]piperazine;

1-[2R-(2-piperazinylcarboxamido)-3-(2,4-dichlorophenyl)propionyl]-4-[2-(1-methyl-2-methoxyethyl)aminomethyl-4-(trifluoromethyl)phenyl]piperazine;

1-[2R-(2-piperidinylcarbonyl)-3-(2,4-dichlorophenyl)propionyl]-4-[2-(1-methyl-2-methoxyethyl)aminomethyl-4-(trifluoromethyl)phenyl]piperazine;

1-[2R-(3-piperidinylcarbonyl)-3-(2,4-dichlorophenyl)propionyl]-4-[2-(1-methyl-2-methoxyethyl)aminomethyl-4-(trifluoromethyl)phenyl]piperazine;

1-[2R-(4-piperidinylcarbonyl)-3-(2,4-dichlorophenyl)propionyl]-4-[2-(1-methyl-2-methoxyethyl)aminomethyl-4-(trifluoromethyl)phenyl]piperazine;

1-[2R-(2-methylaminoacetamido)-3-(2,4-dichlorophenyl)propionyl]-2R-methyl-4-[2-(1-methyl-2-methoxyethyl)aminomethyl-4-(trifluoromethyl)phenyl]piperazine;

1-[2R-(2-methylaminoacetamido)-3-(2,4-dichlorophenyl)propionyl]-2S-methyl-4-[2-(1-methyl-2-methoxyethyl)aminomethyl-4-(trifluoromethyl)phenyl]piperazine;

1-[2R-(2-methylaminoacetamido)-3-(2,4-dichlorophenyl)propionyl]-2R-hydroxymethyl-4-[2-(1-methyl-2-methoxyethyl)aminomethyl-4-(trifluoromethyl)phenyl]piperazine;

1-[2R-(2-methylaminoacetamido)-3-(2,4-dichlorophenyl)propionyl]-2S-hydroxymethyl-4-[2-(1-methyl-2-methoxyethyl)aminomethyl-4-(trifluoromethyl)phenyl]piperazine;

1-[2R-(2-methylaminoacetamido)-3-(4-methoxyphenyl)propionyl]-4-[2-(1-methyl-2-methoxyethyl)aminomethyl-4-(trifluoromethyl)phenyl]piperazine;

1-[2R-(2-methylaminoacetamido)-3-(4-chlorophenyl)propionyl]-4-[2-(1-methyl-2-methoxyethyl)aminomethyl-4-(trifluoromethyl)phenyl]piperazine;

1-[2R-(2-methylaminoacetamido)-3-(4-bromophenyl)propionyl]-4-[2-(1-methyl-2-methoxyethyl)aminomethyl-4-(trifluoromethyl)phenyl]piperazine;

1-[2R-(2-methylaminoacetamido)-3-(2-chloro-4-methoxyphenyl)propionyl]-4-[2-(1-methyl-2-methoxyethyl)aminomethyl-4-(trifluoromethyl)phenyl]piperazine;

1-[2R-(2-methylaminoacetamido)-3-(4-chloro-2-methoxyphenyl)propionyl]-4-[2-(1-methyl-2-methoxyethyl)aminomethyl-4-(trifluoromethyl)phenyl]piperazine;

1-[2R-(2-methylaminoacetamido)-3-(2-methyl-4-methoxyphenyl)propionyl]-4-[2-(1-methyl-2-methoxyethyl)aminomethyl-4-(trifluoromethyl)phenyl]piperazine;

1-[2R-(2-methylaminoacetamido)-3-(2-methyl-4-chlorophenyl)propionyl]-4-[2-(1-methyl-2-methoxyethyl)aminomethyl-4-(trifluoromethyl)phenyl piperazine;

1-[2R-(2-methylaminoacetamido)-3-(1-naphthyl)propionyl]-4-[2-(1-methyl-2-methoxyethyl)aminomethyl-4-(trifluoromethyl)phenyl]piperazine;

1-[2R-(2-methylaminoacetamido)-3-(2-naphthyl)propionyl]-4-[2-(1-methyl-2-methoxyethyl)aminomethyl-4-(trifluoromethyl)phenyl]piperazine;

1-[3-(2,4-dichlorophenyl)propionyl]-4-[2-(N-methyl-2-methoxyethyl)-aminomethyl-4-(trifluoromethyl)phenyl]piperazine;

1-[3-(2,4-dichlorophenyl)propionyl]-4-[2-(2-methoxyphenethyl)aminomethyl-4-(trifluoromethyl)phenyl]piperazine;

1-[3-(2,4-dichlorophenyl)propionyl]-4-[2-(2-fluorophenethyl)aminomethyl-4-(trifluoromethyl)phenyl]piperazine;

1-[3-(2,4-dichlorophenyl)propionyl]-4-[2-(2-thiophenethyl)aminomethyl-4-(trifluoromethyl)phenyl]piperazine;

1-[3-(2,4-dichlorophenyl)propionyl]-4-[2-(1-methyl-2-methoxyethyl)-aminomethyl-4-(trifluoromethyl)phenyl]piperazine;

1-[3-(2,4-dichlorophenyl)propionyl]-4-{2-[1-(methoxyethylamino)ethyl]-4-(trifluoromethyl)phenyl}piperazine;

1-[3-(2,4-dichlorophenyl)propionyl]-4-{2-[1-bis(methoxyethyl)aminoethyl]-4-(trifluoromethyl)phenyl}piperazine;

1-[3-(2,4-dichlorophenyl)propionyl]-4-{2-[1-amino-2-metylbutyl]-4-(trifluoromethyl)phenyl}piperazine;

1-[3-(2,4-dichlorophenyl)propionyl]-4-{2-[1-methylamino-2-metylbutyl]-4-(trifluoromethyl)phenyl}piperazine;

1-[3-(2,4-dichlorophenyl)propionyl]-4-{2-[1-ethylamino-2-metylbutyl]-4-(trifluoromethyl)phenyl}piperazine;

1-[3-(2,4-dichlorophenyl)propionyl]-4-{2-[1-hydroxyethylamino-2-metylbutyl]-4-(trifluoromethyl)phenyl}piperazine;

1-[3-(2,4-dichlorophenyl)propionyl]-4-{2-[1-aminoethylamino-2-metylbutyl]-4-(trifluoromethyl)phenyl}piperazine;

1-[3-(2,4-dichlorophenyl)propionyl]-4-{2-[1-amino-3-metylbutyl]-4-(trifluoromethyl)phenyl}piperazine;

1-[2-methyl-3-(2,4-dichlorophenyl)propionyl]-4-{2-[1-amino-3-metylbutyl]-4-(trifluoromethyl)phenyl}piperazine;

1-[2-ethyl-3-(2,4-dichlorophenyl)propionyl]-4-{2-[1-amino-3-metylbutyl]-(trifluoromethyl)phenyl}piperazine;

1-[2-isopropyl-3-(2,4-dichlorophenyl)propionyl]-4-{2-[1-amino-3-metylbutyl]-4-(trifluoromethyl)phenyl}piperazine;

1-[2-amino-3-(2,4-dichlorophenyl)propionyl]-4-{2-[1-amino-3-metylbutyl]-4-(trifluoromethyl)phenyl}piperazine;

1-[2-dimethylamino-3-(2,4-dichlorophenyl)propionyl]-4-{2-[1-amino-3-metylbutyl]-4-(trifluoromethyl)phenyl}piperazine;

1-[2-dimethylamino-3-(2,4-dichlorophenyl)propionyl]-4-{2-[1-methylamino-3-metylbutyl]-4-(trifluoromethyl)phenyl}piperazine;

1-[2-bis(2-pyridyl)amino-3-(2,4-dichlorophenyl)propionyl]-4-{2-[1-amino-3-metylbutyl]phenyl}piperazine;

1-[3-(2,4-dichlorophenyl)propionyl]-4-{2-[1-methylamino-3-metylbutyl]-4-(trifluoromethyl)phenyl}piperazine;

1-[3-(2,4-dichlorophenyl)propionyl]-4-{2-[1-ethylamino-3-metylbutyl]-4-(trifluoromethyl)phenyl}piperazine;

1-[3-(2,4-dichlorophenyl)propionyl]-4-{2-[1-aminoethylamino-3-metylbutyl]-4-(trifluoromethyl)phenyl}piperazine;

1-[3-(2,4-dichlorophenyl)propionyl]-4-{2-[1-amino-3-metylbutyl]-4-chlorophenyl}piperazine;

1-[3-(2,4-dichlorophenyl)propionyl]-4-{2-[1-amino-3-metylbutyl]-4-bromophenyl}piperazine;

1-[3-(2,4-dichlorophenyl)propionyl]-4-{2-[1-amino-3-metylbutyl]-4-thiophenyl)phenyl}piperazine;

1-[3-(2,4-dichlorophenyl)propionyl]-4-{2-[1-amino-3-metylbutyl]-4-(3-thiophenyl)phenyl}piperazine;

1-[3-(2,4-dichlorophenyl)propionyl]-4-{2-[1-amino-3-metylbutyl]-2-chloropyridyl)phenyl}piperazine;

1-[2-(2-Oxo-1-pyrrolidinyl)-3-(2,4-dichlorophenyl)propionyl]-4-[2-(2-cyanoethyl)aminomethylphenyl]piperazine;

1-[2-(2-Oxo-1-pyrrolidinyl)-3-(2,4-dichlorophenyl)propionyl]-4-[2-(N-methyl-2-methoxyethyl)aminomethylphenyl]piperazine;

1-[2-(2-Oxo-1-pyrrolidinyl)-3-(2,4-dichlorophenyl)propionyl]-4-[2-(1R-methyl-methoxyethyl)aminomethylphenyl]piperazine;

1-[2-(2-Oxo-1-pyrrolidinyl)-3-(2,4-dichlorophenyl)propionyl]-4-[2-(1S-methyl-2-methoxyethyl)aminomethylphenyl]piperazine;

1-[2-(2-Oxo-1-pyrrolidinyl)-3-(2,4-dichlorophenyl)propionyl]-4-[2-(1R-methyl-2-methoxyethyl)aminomethyl-4-(trifluoromethyl)phenyl]piperazine;

1-[2-(2-Oxo-1-pyrrolidinyl)-3-(2,4-dichlorophenyl)propionyl]-4-[2-(1S-methyl-2-methoxyethyl)aminomethyl-4-(trifluoromethyl)phenyl]piperazine;

1-[2-(2-Oxo-1-pyrrolidinyl)-3-(2,4-dichlorophenyl)propionyl]-4-[2-(1-methyl-2-methoxyethyl)aminomethyl-5-(trifluoromethyl)phenyl]piperazine;

1-[2-(2-Oxo-1-pyrrolidinyl)-3-(2,4-dichlorophenyl)propionyl]-4-[2-(1-methyl-2-methoxyethyl)aminomethyl-6-(trifluoromethyl)phenyl]piperazine;

1-[2-(2-Oxo-1-pyrrolidinyl)-3-(2,4-dichlorophenyl)propionyl]-4-[2-(1-methyl-2-methoxyethyl)aminomethyl-4-chlorophenyl]piperazine;

1-[2-(2-Oxo-1-pyrrolidinyl)-3-(2,4-dichlorophenyl)propionyl]-4-[2-(1-methyl-2-methoxyethyl)aminomethyl-3-fluorophenyl]piperazine;

1-[2-(2-Oxo-1-pyrrolidinyl)-3-(2,4-dichlorophenyl)propionyl]-4-[2-(1-methyl-2-methoxyethyl)aminomethyl-4-fluorophenyl]piperazine;

1-[2-(2-Oxo-1-pyrrolidinyl)-3-(2,4-dichlorophenyl)propionyl]-4-[2-(1-methyl-2-methoxyethyl)aminomethyl-5-fluorophenyl]piperazine;

1-[2-(2-Oxo-1-pyrrolidinyl)-3-(2,4-dichlorophenyl)propionyl]-4-[2R-(2-methyl-2-methoxyethylamino)ethyl-4-(trifluoromethyl)phenyl]piperazine;

1-[2-(2-Oxo-1-pyrrolidinyl)-3-(2,4-dichlorophenyl)propionyl]-4-[2S-(2-methyl-2-methoxyethylamino)ethyl-4-(trifluoromethyl)phenyl]piperazine;

1-[2-(2-Oxo-1-pyrrolidinyl)-3-(2,4-dichlorophenyl)propionyl]-4-[2-(1R-amino-3-methylbutyl)phenyl]piperazine;

1-[2-(2-Oxo-1-pyrrolidinyl)-3-(2,4-dichlorophenyl)propionyl]-4-[2-(1S-amino-3-methylbutyl)phenyl]piperazine;

1-[2-(2-Oxo-1-pyrrolidinyl)-3-(2,4-dichlorophenyl)propionyl]-4-[2-(1-amino-3-methylbutyl)-4-(trifluoromethyl)phenyl]piperazine;

1-[2-(2-Oxo-1-pyrrolidinyl)-3-(2,4-dichlorophenyl)propionyl]-4-[2-(1-amino-3-methylbutyl)-4-chlorophenyl]piperazine;

1-[2-(2-Oxo-1-pyrrolidinyl)-3-(2,4-dichlorophenyl)propionyl]-4-[2-(1-amino-3-methylbutyl)-4-fluorophenyl]piperazine;

1-[2-(2-Oxo-1-pyrrolidinyl)-3-(2,4-dichlorophenyl)propionyl]-4-[2-(1-amino-3-methylbutyl)-6-(trifluoromethyl)phenyl]piperazine;

1-[2-(2-Oxo-1-pyrrolidinyl)-3-(2,4-dichlorophenyl)propionyl]-4-[2-(1-amino-3-methylbutyl)-6-fluorophenyl]piperazine;

1-[2-(2-Oxo-1-pyrrolidinyl)-3-(2,4-dichlorophenyl)propionyl]-4-[2-(1-amino-3-methylbutyl)-4-(trifluoromethyl)phenyl]piperazine;

1-[2-(2-Oxo-3-amino-1-pyrrolidinyl)-3-(2,4-dichlorophenyl)propionyl]-4-[2-(1-amino-3-methylbutyl)-4-(trifluoromethyl)phenyl]piperazine;

1-[2-(2-Oxo-3-methylamino-1-pyrrolidinyl)-3-(2,4-dichlorophenyl)propionyl]-4-[2-(1-amino-3-methylbutyl)-4-(trifluoromethyl)phenyl]piperazine;

1-[2-(2-Oxo-3-dimethylamino-1-pyrrolidinyl)-3-(2,4-dichlorophenyl)propionyl]-4-[2-(1-amino-3-methylbutyl)-4-(trifluoromethyl)phenyl]piperazine;

1-[2-(2-Oxo-1-pyrrolidinyl)-3-(2,4-dichlorophenyl)propionyl]-4-[2-(1-amino-2-methylbutyl)-4-(trifluoromethyl)phenyl]piperazine;

1-[2-(2-Oxo-1-pyrrolidinyl)-3-(2,4-dichlorophenyl)propionyl]-4-[2-(1-methylamino-3-methylbutyl)-6-fluorophenyl]piperazine;

1-[2-(2-Oxo-1-pyrrolidinyl)-3-(2,4-dichlorophenyl)propionyl]-4-[2-(1-ethylamino-3-methylbutyl)-6-fluorophenyl]piperazine;

1-[2-(2-Oxo-1-pyrrolidinyl)-3-(2,4-dichlorophenyl)propionyl]-4-[2-(1-aminocarbonylmethylamino-3-methylbutyl)-6-fluorophenyl]piperazine;

1-[2-(2-Oxo-1-pyrrolidinyl)-3-(2,4-dichlorophenyl)propionyl]-4-[2-(1-hydroxyethylamino-3-methylbutyl)-6-fluorophenyl]piperazine;

1-[2-(2-Oxo-1-pyrrolidinyl)-3-(2,4-dichlorophenyl)propionyl]-4-[2-(1-aminoethylamino-3-methylbutyl)-6-fluorophenyl]piperazine;

1-[2-(2-Oxo-1-pyrrolidinyl)-3-(2,4-dichlorophenyl)propionyl]-4-[2-(1-methylaminoethylamino-3-methylbutyl)-6-fluorophenyl]piperazine;

1-[2-(2-Oxo-1-pyrrolidinyl)-3-(2,4-dichlorophenyl)propionyl]-4-[2-(1-dimethylaminoethylamino-3-methylbutyl)-6-fluorophenyl]piperazine;

1-[2-(2-Oxo-1-pyrrolidinyl)-3-(2,4-dichlorophenyl)propionyl]-4-[2-(1-aminopropylamino-3-methylbutyl)-6-fluorophenyl]piperazine;

1-[2-(2-Oxo-1-pyrrolidinyl)-3-(2,4-dichlorophenyl)propionyl]-4-[2-(1-methylamino-3-methylbutyl)-4-fluorophenyl]piperazine;

1-[2-(2-Oxo-1-pyrrolidinyl)-3-(2,4-dichlorophenyl)propionyl]-4-[2-(1-ethylamino-3-methylbutyl)-4-fluorophenyl]piperazine;

1-[2-(2-Oxo-1-pyrrolidinyl)-3-(2,4-dichlorophenyl)propionyl]-4-[2-(1-aminocarbonylmethylamino-3-methylbutyl)-4-fluorophenyl]piperazine;

1-[2-(2-Oxo-1-pyrrolidinyl)-3-(2,4-dichlorophenyl)propionyl]-4-[2-(1-hydroxyethylamino-3-methylbutyl)-4-fluorophenyl]piperazine;

1-[2-(2-Oxo-1-pyrrolidinyl)-3-(2,4-dichlorophenyl)propionyl]-4-[2-(1-aminoethylamino-3-methylbutyl)-4-fluorophenyl]piperazine;

1-[2-(2-Oxo-1-pyrrolidinyl)-3-(2,4-dichlorophenyl)propionyl]-4-[2-(1-methylaminoethylamino-3-methylbutyl)-4-fluorophenyl]piperazine;

1-[2-(2-Oxo-1-pyrrolidinyl)-3-(2,4-dichlorophenyl)propionyl]-4-[2-(1-dimethylaminoethylamino-3-methylbutyl)-4-fluorophenyl]piperazine;

1-[2-(2-Oxo-1-pyrrolidinyl)-3-(2,4-dichlorophenyl)propionyl]-4-[2-(1-aminopropylamino-3-methylbutyl)-4-fluorophenyl]piperazine;

1-[2-(2-Oxo-1-oxazolidinyl)-3-(2,4-dichlorophenyl)propionyl]-4-[2-(2-cyanoethyl)aminomethylphenyl]piperazine;

1-[2-(2-Oxo-1-oxazolidinyl)-3-(2,4-dichlorophenyl)propionyl]-4-[2-(N-methyl-2-methoxyethyl)aminomethylphenyl]piperazine;

1-[2-(2-Oxo-1-oxazolidinyl)-3-(2,4-dichlorophenyl)propionyl]-4-[2-(1R-methyl-2-methoxyethyl)aminomethylphenyl]piperazine;

1-[2-(2-Oxo-1-oxazolidinyl)-3-(2,4-dichlorophenyl)propionyl]-4-[2-(1S-methyl-2-methoxyethyl)aminomethylphenyl]piperazine;

1-[2-(2-Oxo-1-oxazolidinyl)-3-(2,4-dichlorophenyl)propionyl]-4-[2-(1R-methyl-2-methoxyethyl)aminomethyl-4-(trifluoromethyl)phenyl]piperazine;

1-[2-(2-Oxo-1-oxazolidinyl)-3-(2,4-dichlorophenyl)propionyl]-4-[2-(1S-methyl-2-methoxyethyl)aminomethyl-4-(trifluoromethyl)phenyl]piperazine;

1-[2-(2-Oxo-1-oxazolidinyl)-3-(2,4-dichlorophenyl)propionyl]-4-[2-(1-methyl-2-methoxyethyl)aminomethyl-5-(trifluoromethyl)phenyl]piperazine;

1-[2-(2-Oxo-1-oxazolidinyl)-3-(2,4-dichlorophenyl)propionyl]-4-[2-(1-methyl-2-methoxyethyl)aminomethyl-6-(trifluoromethyl)phenyl]piperazine;

1-[2-(2-Oxo-1-oxazolidinyl)-3-(2,4-dichlorophenyl)propionyl]-4-[2-(1-methyl-2-methoxyethyl)aminomethyl-4-chlorophenyl]piperazine;

1-[2-(2-Oxo-1-oxazolidinyl)-3-(2,4-dichlorophenyl)propionyl]-4-[2-(1-methyl-2-methoxyethyl)aminomethyl-3-fluorophenyl]piperazine;

1-[2-(2-Oxo-1-oxazolidinyl)-3-(2,4-dichlorophenyl)propionyl]-4-[2-(1-methyl-2-methoxyethyl)aminomethyl-4-fluorophenyl]piperazine;

1-[2-(2-Oxo-1-oxazolidinyl)-3-(2,4-dichlorophenyl)propionyl]-4-[2-(1-methyl-2-methoxyethyl)aminomethyl-5-fluorophenyl]piperazine;

1-[2-(2-Oxo-1-oxazolidinyl)-3-(2,4-dichlorophenyl)propionyl]-4-[2R-(2-methoxyethylamino)ethyl-4-(trifluoromethyl)phenyl]piperazine;

1-[2-(2-Oxo-1-oxazolidinyl)-3-(2,4-dichlorophenyl)propionyl]-4-[2S-(2-methoxyethylamino)ethyl-4-(trifluoromethyl)phenyl]piperazine;

1-[2-(2-Oxo-1-oxazolidinyl)-3-(2,4-dichlorophenyl)propionyl]-4-[2-(1R-amino-3-methylbutyl)phenyl]piperazine;

1-[2-(2-Oxo-1-oxazolidinyl)-3-(2,4-dichlorophenyl)propionyl]-4-[2-(1S-amino-3-methylbutyl)phenyl]piperazine;

1-[2-(2-Oxo-1-oxazolidinyl)-3-(2,4-dichlorophenyl)propionyl]-4-[2-(1-amino-3-methylbutyl)-4-(trifluoromethyl)phenyl]piperazine;

1-[2-(2-Oxo-1-oxazolidinyl)-3-(2,4-dichlorophenyl)propionyl]-4-[2-(1-amino-3-methylbutyl)-4-chlorophenyl]piperazine;

1-[2-(2-Oxo-1-oxazolidinyl)-3-(2,4-dichlorophenyl)propionyl]-4-[2-(1-amino-3-methylbutyl)-4-fluorophenyl]piperazine;

1-[2-(2-Oxo-1-oxazolidinyl)-3-(2,4-dichlorophenyl)propionyl]-4-[2-(1-amino-3-methylbutyl)-6-(trifluoromethyl)phenyl]piperazine;

1-[2-(2-Oxo-1-oxazolidinyl)-3-(2,4-dichlorophenyl)propionyl]-4-[2-(1-amino-3-methylbutyl)-4-fluorophenyl]piperazine;

1-[2-(2-Oxo-1-oxazolidinyl)-3-(2,4-dichlorophenyl)propionyl]-4-[2-(1-amino-3-methylbutyl)-4-(trifluoromethyl)phenyl]piperazine;

1-[2-(2-Oxo-1-imidazolidinyl)-3-(2,4-dichlorophenyl)propionyl]-4-[2-(2-cyanoethyl)aminomethylphenyl]piperazine;

1-[2-(2-Oxo-1-imidazolidinyl)-3-(2,4-dichlorophenyl)propionyl]-4-[2-(N-methyl-2-methoxyethyl)aminomethylphenyl]piperazine;

1-[2-(2-Oxo-1-imidazolidinyl)-3-(2,4-dichlorophenyl)propionyl]-4-[2-(1R-methyl-2-methoxyethyl)aminomethylphenyl]piperazine;

1-[2-(2-Oxo-1-imidazolidinyl)-3-(2,4-dichlorophenyl)propionyl]-4-[2-(1S-methyl-2-methoxyethyl)aminomethylphenyl]piperazine;

1-[2-(2-Oxo-1-imidazolidinyl)-3-(2,4-dichlorophenyl)propionyl]-4-[2-(1R-methyl-2-methoxyethyl)aminomethyl-4-(trifluoromethyl)phenyl]piperazine;

1-[2-(2-Oxo-1-imidazolidinyl)-3-(2,4-dichlorophenyl)propionyl]-4-[2-(1S-methyl-2-methoxyethyl)aminomethyl-4-(trifluoromethyl)phenyl]piperazine;

1-[2-(2-Oxo-1-imidazolidinyl)-3-(2,4-dichlorophenyl)propionyl]-4-[2-(1-methyl-2-methoxyethyl)aminomethyl-5-(trifluoromethyl)phenyl]piperazine;

1-[2-(2-Oxo-1-imidazolidinyl)-3-(2,4-dichlorophenyl)propionyl]-4-[2-(1-methyl-2-methoxyethyl)aminomethyl-6-(trifluoromethyl)phenyl]piperazine;

1-[2-(2-Oxo-1-imidazolidinyl)-3-(2,4-dichlorophenyl)propionyl]-4-[2-(1-methyl-2-methoxyethyl)aminomethyl-4-chlorophenyl]piperazine;

1-[2-(2-Oxo-1-imidazolidinyl)-3-(2,4-dichlorophenyl)propionyl]-4-[2-(1-methyl-2-methoxyethyl)aminomethyl-3-fluorophenyl]piperazine;

1-[2-(2-Oxo-1-imidazolidinyl)-3-(2,4-dichlorophenyl)propionyl]-4-[2-(1-methyl-2-methoxyethyl)aminomethyl-4-fluorophenyl]piperazine;

1-[2-(2-Oxo-1-imidazolidinyl)-3-(2,4-dichlorophenyl)propionyl]-4-[2-(1-methyl-2-methoxyethyl)aminomethyl-5-fluorophenyl]piperazine;

1-[2-(2-Oxo-1-imidazolidinyl)-3-(2,4-dichlorophenyl)propionyl]-4-[2R-(2-methoxyethylamino)ethyl-4-(trifluoromethyl)phenyl]piperazine;

1-[2-(2-Oxo-1-imidazolidinyl)-3-(2,4-dichlorophenyl)propionyl]-4-[2S-(2-methoxyethylamino)ethyl-4-(trifluoromethyl)phenyl]piperazine;

1-[2-(2-Oxo-1-imidazolidinyl)-3-(2,4-dichlorophenyl)propionyl]-4-[2-(1R-amino-3-methylbutyl)phenyl]piperazine;

1-[2-(2-Oxo-1-imidazolidinyl)-3-(2,4-dichlorophenyl)propionyl]-4-[2-(1S-amino-3-methylbutyl)phenyl]piperazine;

1-[2-(2-Oxo-1-imidazolidinyl)-3-(2,4-dichlorophenyl)propionyl]-4-[2-(1-amino-3-methylbutyl)-4-(trifluoromethyl)phenyl]piperazine;

1-[2-(2-Oxo-1-imidazolidinyl)-3-(2,4-dichlorophenyl)propionyl]-4-[2-(1-amino-3-methylbutyl)-4-chlorophenyl]piperazine;

1-[2-(2-Oxo-1-imidazolidinyl)-3-(2,4-dichlorophenyl)propionyl]-4-[2-(1-amino-3-methylbutyl)-4-fluorophenyl]piperazine;

1-[2-(2-Oxo-1-imidazolidinyl)-3-(2,4-dichlorophenyl)propionyl]-4-[2-(1-amino-3-methylbutyl)-6-(trifluoromethyl)phenyl]piperazine;

1-[2-(2-Oxo-1-imidazolidinyl)-3-(2,4-dichlorophenyl)propionyl]-4-[2-(1-amino-3-methylbutyl)-4-fluorophenyl]piperazine;

1-[2-(2-Oxo-1-imidazolidinyl)-3-(2,4-dichlorophenyl)propionyl]-4-[2-(1-amino-3-methylbutyl)-4-(trifluoromethyl)phenyl]piperazine;

1-[2-(2-Oxo-3-methyl-1-imidazolidinyl)-3-(2,4-dichlorophenyl)propionyl]-4-[2-(1-amino-3-methylbutyl)-4-(trifluoromethyl)phenyl]piperazine;

1-[2-(2-Oxo-3-ethyl-1-imidazolidinyl)-3-(2,4-dichlorophenyl)propionyl]-4-[2-(1-amino-3-methylbutyl)-4-(trifluoromethyl)phenyl]piperazine;

1-[2-(2-Oxo-3-hydroxyethyl-1-imidazolidinyl)-3-(2,4-dichlorophenyl)propionyl]-4-[2-(1-amino-3-methylbutyl)-4-(trifluoromethyl)phenyl]-piperazine;

1-[2-(2-Oxo-3-aminoethyl-1-imidazolidinyl)-3-(2,4-dichlorophenyl)propionyl]-4-[2-(1-amino-3-methylbutyl)-4-(trifluoromethyl)phenyl]piperazine;

1-[2-(2-Oxo-3-methylaminoethyl-1-imidazolidinyl)-3-(2,4-dichlorophenyl)propionyl]-4-[2-(1-amino-3-methylbutyl)-4-(trifluoromethyl)phenyl]piperazine;

1-[2-(2-Oxo-3-dimethylaminoethyl-1-imidazolidinyl)-3-(2,4-dichlorophenyl)propionyl]-4-[2-(1-amino-3-methylbutyl)-4-(trifluoromethyl)phenyl]piperazine;

1-[2-(2-Oxo-1-piperazinyl)-3-(2,4-dichlorophenyl)propionyl]-4-[2-(2-cyanoethyl)aminomethylphenyl]piperazine;

1-[2-(2-Oxo-1-piperazinyl)-3-(2,4-dichlorophenyl)propionyl]-4-[2-(N-methyl-2-methoxyethyl)aminomethylphenyl]piperazine;

1-[2-(2-Oxo-1-piperazinyl)-3-(2,4-dichlorophenyl)propionyl]-4-[2-(1R-methyl-2-methoxyethyl)aminomethylphenyl]piperazine;

1-[2-(2-Oxo-1-piperazinyl)-3-(2,4-dichlorophenyl)propionyl]-4-[2-(1S-methyl-2-methoxyethyl)aminomethylphenyl]piperazine;

1-[2-(2-Oxo-1-piperazinyl)-3-(2,4-dichlorophenyl)propionyl]-4-[2-(1R-methyl-2-methoxyethyl)aminomethyl-4-(trifluoromethyl)phenyl]piperazine;

1-[2-(2-Oxo-1-piperazinyl)-3-(2,4-dichlorophenyl)propionyl]-4-[2-(1S-methyl-2-methoxyethyl)aminomethyl-4-(trifluoromethyl)phenyl]piperazine;

1-[2-(2-Oxo-1-piperazinyl)-3-(2,4-dichlorophenyl)propionyl]-4-[2-(1-methyl-2-methoxyethyl)aminomethyl-5-(trifluoromethyl)phenyl]piperazine;

1-[2-(2-Oxo-1-piperazinyl)-3-(2,4-dichlorophenyl)propionyl]-4-[2-(1-methyl-2-methoxyethyl)aminomethyl-6-(trifluoromethyl)phenyl]piperazine;

1-[2-(2-Oxo-1-piperazinyl)-3-(2,4-dichlorophenyl)propionyl]-4-[2-(1-methyl-2-methoxyethyl)aminomethyl-4-chlorophenyl]piperazine;

1-[2-(2-Oxo-1-piperazinyl)-3-(2,4-dichlorophenyl)propionyl]-4-[2-(1-methyl-2-methoxyethyl)aminomethyl-3-fluorophenyl]piperazine;

1-[2-(2-Oxo-1-piperazinyl)-3-(2,4-dichlorophenyl)propionyl]-4-[2-(1-methyl-2-methoxyethyl)aminomethyl-4-fluorophenyl]piperazine;

1-[2-(2-Oxo-1-piperazinyl)-3-(2,4-dichlorophenyl)propionyl]-4-[2-(1-methyl-2-methoxyethyl)aminomethyl-5-fluorophenyl]piperazine;

1-[2-(2-Oxo-1-piperazinyl)-3-(2,4-dichlorophenyl)propionyl]-4-[2R-(2-methoxyethylamino)ethyl-4-(trifluoromethyl)phenyl]piperazine;

1-[2-(2-Oxo-1-piperazinyl)-3-(2,4-dichlorophenyl)propionyl]-4-[2S-(2-methoxyethylamino)ethyl-4-(trifluoromethyl)phenyl]piperazine;

1-[2-(2-Oxo-1-piperazinyl)-3-(2,4-dichlorophenyl)propionyl]-4-[2-(1R-amino-3-methylbutyl)phenyl]piperazine;

1-[2-(2-Oxo-1-piperazinyl)-3-(2,4-dichlorophenyl)propionyl]-4-[2-(1S-amino-3-methylbutyl)phenyl]piperazine;

1-[2-(2-Oxo-1-piperazinyl)-3-(2,4-dichlorophenyl)propionyl]-4-[2-(1-amino-3-methylbutyl)-4-(trifluoromethyl)phenyl]piperazine;

1-[2-(2-Oxo-1-piperazinyl)-3-(2,4-dichlorophenyl)propionyl]-4-[2-(1-amino-3-methylbutyl)-4-chlorophenyl]piperazine;

1-[2-(2-Oxo-1-piperazinyl)-3-(2,4-dichlorophenyl)propionyl]-4-[2-(1-amino-3-methylbutyl)-4-fluorophenyl]piperazine;

1-[2-(2-Oxo-1-piperazinyl)-3-(2,4-dichlorophenyl)propionyl]-4-[2-(1-amino-3-methylbutyl)-6-(trifluoromethyl)phenyl]piperazine;

1-[2-(2-Oxo-1-piperazinyl)-3-(2,4-dichlorophenyl)propionyl]-4-[2-(1-amino-3-methylbutyl)-4-fluorophenyl]piperazine;

1-[2-(2-Oxo-1-piperazinyl)-3-(2,4-dichlorophenyl)propionyl]-4-[2-(1-amino-3-methylbutyl)-4-(trifluoromethyl)phenyl]piperazine;

1-[2-(2-Oxo-4-methyl-1-piperazinyl)-3-(2,4-dichlorophenyl)propionyl]-4-[2-(1-amino-3-methylbutyl)-4-(trifluoromethyl)phenyl]piperazine;

1-[2-(2-Oxo-4-ethyl-1-piperazinyl)-3-(2,4-dichlorophenyl)propionyl]-4-[2-(1-amino-3-methylbutyl)-4-(trifluoromethyl)phenyl]piperazine;

1-[2-(2-Oxo-4-isopropyl-1-piperazinyl)-3-(2,4-dichlorophenyl)propionyl]-4-[2-(1-amino-3-methylbutyl)-4-(trifluoromethyl)phenyl]piperazine;

1-[2-(2-Oxo-4-hydroxyethyl-1-piperazinyl)-3-(2,4-dichlorophenyl)propionyl]-4-[2-(1-amino-3-methylbutyl)-4-(trifluoromethyl)phenyl]piperazine;

1-[2-(2-Oxo-4-aminoethyl-1-piperazinyl)-3-(2,4-dichlorophenyl)propionyl]-4-[2-(1-amino-3-methylbutyl)-4-(trifluoromethyl)phenyl]piperazine;

1-[2-(2-Oxo-4-methylaminoethyl-1-piperazinyl)-3-(2,4-dichlorophenyl)propionyl]-4-[2-(1-amino-3-methylbutyl)-4-(trifluoromethyl)phenyl]-piperazine;

1-[2-(2-Oxo-4-dimethylaminoethyl-1-piperazinyl)-3-(2,4-dichlorophenyl)propionyl]-4-[2-(1-amino-3-methylbutyl)-4-(trifluoromethyl)phenyl]-piperazine;

1-[2-(1-pyrrolyl)-3-(2,4-dichlorophenyl)propionyl]-4-[2-(1-amino-3-methylbutyl)-4-(trifluoromethyl)phenyl]piperazine;

1-[2-(1-imidazolyl)-3-(2,4-dichlorophenyl)propionyl]-4-[2-(1-amino-3-methylbutyl)-4-(trifluoromethyl)phenyl]piperazine;

1-[2-(1-triazolyl)-3-(2,4-dichlorophenyl)propionyl]-4-[2-(1-amino-3-methylbutyl)-4-(trifluoromethyl)phenyl]piperazine;

1-[2-(4-triazolyl)-3-(2,4-dichlorophenyl)propionyl]-4-[2-(1-amino-3-methylbutyl)-4-(trifluoromethyl)phenyl]piperazine;

1-[2-(1-pyrrolidinyl)-3-(2,4-dichlorophenyl)propionyl]-4-[2-(1-amino-3-methylbutyl)-4-(trifluoromethyl)phenyl]piperazine;

1-[2-(2-oxo-1-piperidinyl)-3-(2,4-dichlorophenyl)propionyl]-4-[2-(1-amino-3-methylbutyl)-4-(trifluoromethyl)phenyl]piperazine;

1-[2-(4-morpholinyl)-3-(2,4-dichlorophenyl)propionyl]-4-[2-(1-amino-3-methylbutyl)-4-(trifluoromethyl)phenyl]piperazine;

1-[2-(3-oxo-4-morpholinyl)-3-(2,4-dichlorophenyl)propionyl]-4-[2-(1-amino-3-methylbutyl)-4-(trifluoromethyl)phenyl]piperazine;

1-[2-(4-thiazinanyl)-3-(2,4-dichlorophenyl)propionyl]-4-[2-(1-amino-3-methylbutyl)-4-(trifluoromethyl)phenyl]piperazine;

1-[2-(3-oxo-4-thiazinanyl)-3-(2,4-dichlorophenyl)propionyl]-4-[2-(1-amino-3-methylbutyl)-4-(trifluoromethyl)phenyl]piperazine;

1-[2-(1,1-dioxo-4-thiazinanyl)-3-(2,4-dichlorophenyl)propionyl]-4-[2-(1-amino-3-methylbutyl)-4-(trifluoromethyl)phenyl]piperazine;

1-[2-(1,1,3-trioxo-4-thiazinanyl)-3-(2,4-dichlorophenyl)propionyl]-4-[2-(1-amino-3-methylbutyl)-4-(trifluoromethyl)phenyl]piperazine;

1-[2-(1-piperidinyl)-3-(2,4-dichlorophenyl)propionyl]-4-[2-(1-amino-3-methylbutyl)-4-(trifluoromethyl)phenyl]piperazine;

1-[2-(1-piperazinyl)-3-(2,4-dichlorophenyl)propionyl]-4-[2-(1-amino-3-methylbutyl)-4-(trifluoromethyl)phenyl]piperazine;

1-[2-(4-methyl-1-piperazinyl)-3-(2,4-dichlorophenyl)propionyl]-4-[2-(1-amino-3-methylbutyl)-4-(trifluoromethyl)phenyl]piperazine;

1-[2-(4-ethyl-1-piperazinyl)-3-(2,4-dichlorophenyl)propionyl]-4-[2-(1-amino-3-methylbutyl)-4-(trifluoromethyl)phenyl]piperazine;

1-[2-(4-benzyl-1-piperazinyl)-3-(2,4-dichlorophenyl)propionyl]-4-[2-(1-amino-3-methylbutyl)-4-(trifluoromethyl)phenyl]piperazine;

1-[2-(4-phenyl-1-piperazinyl)-3-(2,4-dichlorophenyl)propionyl]-4-[2-(1-amino-3-methylbutyl)-4-(trifluoromethyl)phenyl]piperazine;

1-[2-(2-oxo-1-pyridyl)-3-(2,4-dichlorophenyl)propionyl]-4-[2-(1-amino-3-methylbutyl)-4-(trifluoromethyl)phenyl]piperazine;

1-[2-(2-oxo-1-pyrimidyl)-3-(2,4-dichlorophenyl)propionyl]-4-[2-(1-amino-3-methylbutyl)-4-(trifluoromethyl)phenyl]piperazine;

1-[2-(6-oxo-1-pyrimidyl)-3-(2,4-dichlorophenyl)propionyl]-4-[2-(1-amino-3-methylbutyl)-4-(trifluoromethyl)phenyl]piperazine;

1-[2-(3,4,5,6-tetrahydro-2-oxo-1,3-oxazin-3-yl)-3-(2,4-dichlorophenyl)propionyl]-4-[2-(1-amino-3-methylbutyl)-4-(trifluoromethyl)phenyl]piperazine;

1-[2-(3,4,5,6-tetrahydro-2-oxo-1,3-thiazin-3-yl)-3-(2,4-dichlorophenyl)propionyl]-4-[2-(1-amino-3-methylbutyl)-4-(trifluoromethyl)phenyl]piperazine;

1-[2-(3,4,5,6-tetrahydro-2-oxo-1,3-diazin-3-yl)-3-(2,4-dichlorophenyl)propionyl]-4-[2-(1-amino-3-methylbutyl)-4-(trifluoromethyl)phenyl]piperazine;

1-[2-(4-homopiperidinyl)-3-(2,4-dichlorophenyl)propionyl]-4-[2-(1-amino-3-methylbutyl)-4-(trifluoromethyl)phenyl]piperazine;

1-[2-(4-homomorpholinyl)-3-(2,4-dichlorophenyl)propionyl]-4-[2-(1-amino-3-methylbutyl)-4-(trifluoromethyl)phenyl]piperazine;

1-[2-(4-homothiazinyl)-3-(2,4-dichlorophenyl)propionyl]-4-[2-(1-amino-3-methylbutyl)-4-(trifluoromethyl)phenyl]piperazine;

1-[2-(4-homopiperazinyl)-3-(2,4-dichlorophenyl)propionyl]-4-[2-(1-amino-3-methylbutyl)-4-(trifluoromethyl)phenyl]piperazine;

1-[2-(3-oxo-4-homopiperidinyl)-3-(2,4-dichlorophenyl)propionyl]-4-[2-(1-amino-3-methylbutyl)-4-(trifluoromethyl)phenyl]piperazine;

1-[2-(3-oxo-4-homomorpholinyl)-3-(2,4-dichlorophenyl)propionyl]-4-[2-(1-amino-3-methylbutyl)-4-(trifluoromethyl)phenyl]piperazine;

1-[2-(3-oxo-4-homothiazinyl)-3-(2,4-dichlorophenyl)propionyl]-4-[2-(1-amino-3-methylbutyl)-4-(trifluoromethyl)phenyl]piperazine;

1-[2-(3-oxo-4-homopiperazinyl)-3-(2,4-dichlorophenyl)propionyl]-4-[2-(1-amino-3-methylbutyl)-4-(trifluoromethyl)phenyl]piperazine;

1-[2-(5-oxo-4-homopiperidinyl)-3-(2,4-dichlorophenyl)propionyl]-4-[2-(1-amino-3-methylbutyl)-4-(trifluoromethyl)phenyl]piperazine;

1-[2-(5-oxo-4-homomorpholinyl)-3-(2,4-dichlorophenyl)propionyl]-4-[2-(1-amino-3-methylbutyl)-4-(trifluoromethyl)phenyl]piperazine;

1-[2-(5-oxo-4-homothiazinyl)-3-(2,4-dichlorophenyl)propionyl]-4-[2-(1-amino-3-methylbutyl)-4-(trifluoromethyl)phenyl]piperazine;

1-[2-(5-oxo-4-homopiperazinyl)-3-(2,4-dichlorophenyl)propionyl]-4-[2-(1-amino-3-methylbutyl)-4-(trifluoromethyl)phenyl]piperazine;

1-[2-(2-oxo-3-oxazepinyl)-3-(2,4-dichlorophenyl)propionyl]-4-[2-(1-amino-3-methylbutyl)-4-(trifluoromethyl)phenyl]piperazine;

1-[2-(2-oxo-3-thiazepinyl)-3-(2,4-dichlorophenyl)propionyl]-4-[2-(1-amino-3-methylbutyl)-4-(trifluoromethyl)phenyl]piperazine;

1-[2-(2-oxo-3-diazepinyl)-3-(2,4-dichlorophenyl)propionyl]-4-[2-(1-amino-3-methylbutyl)-4-(trifluoromethyl)phenyl]piperazine;

1-[2R-(N-methyl-2-dimethylaminoacetamido)-3-(2,4-dichlorophenyl)propionyl]-4-[2-(1-methyl-2-methoxyethyl)aminomethylphenyl]piperazine;

1-[2R-(N-ethyl-2-dimethylaminoacetamido)-3-(2,4-dichlorophenyl)propionyl]-4-[2-(1-methyl-2-methoxyethyl)aminomethylphenyl]piperazine;

1-[2-(N-acetamido)-3-(2,4-dichlorophenyl)propionyl]-4-{2-[1-amino-3-metylbutyl]-4-(trifluoromethyl)phenyl}piperazine;

1-[2-(N-methyl-acetamido)-3-(2,4-dichlorophenyl)propionyl]-4-{2-[1-amino-3-metylbutyl]-4-(trifluoromethyl)phenyl}piperazine;

1-[2-(N-ethyl-acetamido)-3-(2,4-dichlorophenyl)propionyl]-4-{2-[1-amino-3-metylbutyl]-4-(trifluoromethyl)phenyl}piperazine.

The compounds of the present invention may generally be utilized as the free acid or free base. Alternatively, the compounds of this invention may be used in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Compounds of structure (I) may also possess axial chirality, which may result in atropisomers. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

The compounds of this invention may be evaluated for their ability to bind to a MC receptor by techniques known in this field. For example, a compound may be evaluated for MC receptor binding by monitoring the displacement of an iodonated peptide ligand, typically [¹²⁵I]-NDP-α-MSH, from cells expressing individual melanocortin receptor subtypes. To this end, cells expressing the desired melanocortin receptor are seeded in 96-well microtiter Primaria-coated plates at a density of 50,000 cells per well and allowed to adhere overnight with incubation at 37° C. in 5% CO₂. Stock solutions of test compounds are diluted serially in binding buffer (D-MEM, 1 mg/ml BSA) containing [¹²⁵I]-NDP-α-MSH (10⁵ cpm/ml). Cold NDP-α-MSH is included as a control. Cells are incubated with 50 μl of each test compound concentration for 1 hour at room temperature. Cells are gently washed twice with 250 μl of cold binding buffer and then lysed by addition of 50 μl of 0.5 M NaOH for 20 minutes at room temperature. Protein concentration is determined by Bradford assay and lysates are counted by liquid scintillation spectrometry. Each concentration of test compound is assessed in triplicate. IC₅₀ values are determined by data analysis using appropriate software, such as GraphPad Prizm, and data are plotted as counts of radiolabeled NDP-MSH bound (normalized to protein concentration) versus the log concentration of test compound.

In addition, functional assays of receptor activation have been defined for the MC receptors based on their coupling to G_{S} proteins. In response to POMC peptides, the MC receptors couple to G_{S} and activate adenylyl cyclase resulting in an increase in cAMP production. Melanocortin receptor activity can be measured in HEK293 cells expressing individual melanocortin receptors by direct measurement of cAMP levels or by a reporter gene whose activation is dependent on intracellular cAMP levels. For example, HEK293 cells expressing the desired MC receptor are seeded into 96-well microtiter Primaria-coated plates at a density of 50,000 cells per well and allowed to adhere overnight with incubation at 37° C. in 5% CO₂ Test compounds are diluted in assay buffer composed of D-MEM medium and 0.1 mM isobutylmethylxanthine and assessed for agonist and/or antagonist activity over a range of concentrations along with a control agonist α-MSH. At the time of assay, medium is removed from each well and replaced with test compounds or α-MSH for 30 minutes at 37° C. Cells are harvested by addition of an equal volume of 100% cold ethanol and scraped from the well surface. Cell lysates are centrifuged at 8000×g and the supernatant is recovered and dried under vacuum. The supernatants are evaluated for cAMP using an enzyme-linked immunoassay such as Biotrak, Amersham. EC₅₀ values are determined by data analysis using appropriate software such as GraphPad Prizm, and data are plotted as cAMP produced versus log concentration of compound.

As mentioned above, the compounds of this invention function as ligands to one or more MC receptors, and are thereby useful in the treatment of a variety of conditions or diseases associated therewith. In this manner, the ligands function by altering or regulating the activity of an MC receptor, thereby providing a treatment for a condition or disease associated with that receptor. In this regard, the compounds of this invention have utility over a broad range of therapeutic applications, and may be used to treat disorders or illnesses, including (but not limited to) eating disorders, cachexia, obesity, diabetes, metabolic disorders, inflammation, pain, skin disorders, skin and hair coloration, male and female sexual dysfunction, erectile dysfunction, dry eye, acne and/or Cushing's disease.

The compounds of the present invention may also be used in combination therapy with agents that modify sexual arousal, penile erections, or libido such as sildenafil, yohimbine, apomorphine or other agents. Combination therapy with agents that modify food intake, appetite or metabolism are also included within the scope of this invention. Such agents include, but are not limited to, other MC receptor ligands, ligands of the leptin, NPY, melanin concentrating hormone, serotonin or B₃ adrenergic receptors.

In another embodiment, pharmaceutical compositions containing one or more compounds of this invention are disclosed. For the purposes of administration, the compounds of the present invention may be formulated as pharmaceutical compositions. Pharmaceutical compositions of the present invention comprise a compound of structure (I) and a pharmaceutically acceptable carrier and/or diluent. The compound is present in the composition in an amount which is effective to treat a particular disorder of interest, and preferably with acceptable toxicity to the patient. Typically, the pharmaceutical composition may include a compound of this invention in an amount ranging from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface active agents, binders, and lubricants. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990.

In another embodiment, the present invention provides a method for treating a condition related to an MC receptor. Such methods include administration of a compound of the present invention to a warm-blooded animal in an amount sufficient to treat the condition. In this context, “treat” includes prophylactic administration. Such methods include systemic administration of compound of this invention, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

The following examples are provided for purposes of illustration, not limitation.

### EXAMPLES

Analytical HPLC Columns and Gradients

Analytical HPLC columns were BHK laboratories ODS/0/13 30×75 mm, 51 μm, 120 A; the standard gradient was 1 mL/min 10-90% CH₃CN in water over 2 minutes, then 90% CH₃CN for 1 minute. Constant percentage of 0.1% TFA was added.

Prep HPLC Column

YMC AQ, 5 μm, 120 A20, 20×50 mm cartridges

Analytical HPLC-MS

HP 1100 series: equipped with an auto-sampler, an UV detector (220 nM and 254 nM), a MS detector (electrospray);

HPLC column: YMC ODS AQ, S-5, 5∥, 2.0×50 mm cartridge;

HPLC gradients: 1.5 mL/min, from 10% acetonitrile in water to 90% acetonitrile in water in 2.5 min, maintaining 90% for 1 min.

Prep. HPLC-MS

Gilson HPLC-MS equipped with Gilson 215 auto-sampler/fraction collector, an UV detector and a ThermoFinnigan AQA Single QUAD Mass detector (electrospray);

HPLC column: BHK ODS-O/B, 5 μl, 30×75 mm

HPLC gradients: 35 mL/min, 10% acetonitrile in water to 100% acetonitrile in 7 min, maintaining 100% acetonitrile for 3 min.

Abbreviations:

| | |
|---|---|
| DMSO: | dimethylsulfoxide |
| FMOC: | N-(9-fluorenylmethoxycarbonyl) |
| HOBt: | 1-hydroxybenzotriazole hydrate |
| EDC: | 1-(3-dimethylaminopropyl)-3-ethylcarbodiimide hydrochloride |
| BOC: | tert-butoxycarbonyl |
| DMF: | dimethylformamide |
| TFA: | trifluoroacetic acid |
| HBTU: | O—(1H-Benzotriazol-1-yl)-N,N,N′,N′-tetramethyluronium |
| | hexafluorophosphate |
| Me: | methyl |
| Et: | ethyl |
| Pr: | n-propyl (unless otherwise noted as isopropyl or i-Pr) |
| Bu: | n-butyl (unless otherwise noted as sec-butyl, isobutyl or tert- |
| | butyl, or s-Bu, i-Bu or t-Bu, respectively) |
| c-Pr: | cyclopropyl |
| Ph: | phenyl (—C₆H₅) |
| Bn: | benzyl (—CH₂C₆H₅) |
| Py: | pyridinyl |
| Im: | imidazolyl |
| Ac: | acetyl (i.e., —COCH₃) |

### EXAMPLE 1

### R-3-AMINO-N-[1-(4-CHLOROBENZYL)-2-OXO-2-(4-{2-[(2-THIOPHEN-2-YL-ETHYLAMINO)-METHYL]-PHENYL}-PIPERAZIN-1-YL)-ETHYL]-PROPIONAMIDE

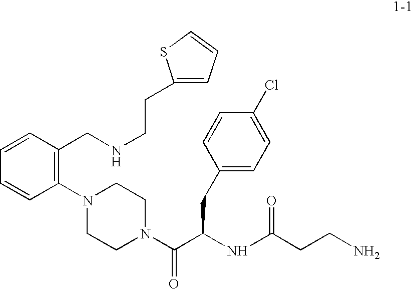

Step 1A: Synthesis of 4-(3-Formyl-phenyl)-piperazine-1-carboxylic acid benzyl ester,

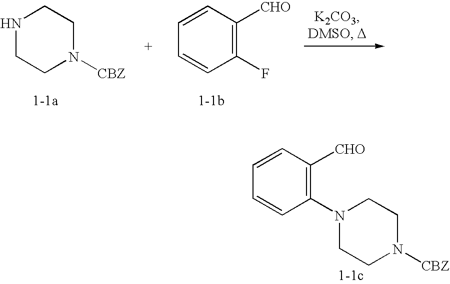

To a solution of benzyl 1-piperazine carboxylate (1-1a, 14.2 g, 64.5 mmol) and 2-fluorobenzaldehyde (1-1b, 8.10 g, 65.3 mmol) in dry degassed DMSO (60 mL) in a pressure tube was added potassium carbonate (12.2 g, 88.3 mmol). The mixture was heated with stirring at 120° C. for 19 h. The mixture was cooled, diluted with ethyl acetate (200 mL), and washed with saturated aqueous ammonium chloride (100 mL). The aqueous layer was extracted with ethyl acetate (100 mL), and the combined organics were dried over sodium sulfate, concentrated in vacuo, and purified by flash column chromatography (10-20% ethyl acetate/dichloromethane) to give the compound 1-1c as a viscous yellow oil (11.0 g, 53%). MS=325.0 ((M+H)⁺).

Step 1B: Reductive Amination, 4-(2-{[tert-Butoxycarbonyl-(2-thiophen-2-yl-ethyl)-amino]-methyl}-phenyl)-piperazine-1-carboxylic acid benzyl ester, 1-1d

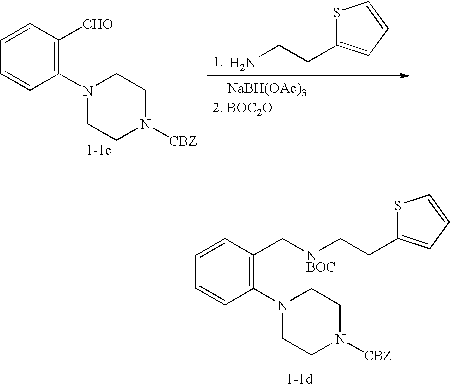

Sodium triacetoxyborohydride (4.50 g, 21.2 mmol) was added in portions to a solution of 1-1c (4.93 g, 15.2 mmol) and 2-thiophen-2-yl-ethylamine (2.04 g, 16.0 mmol) in dry dichloromethane (60 mL) over 5 min. The mixture was stirred for 16 hours, then was quenched with aqueous saturated sodium bicarbonate (30 mL). The mixture was separated, and the aqueous layer was extracted with dichloromethane (2×30 mL). The combined organics were washed with brine (60 mL), dried over magnesium sulfate, and concentrated to give the crude amine (6.79 g). The amine was immediately dissolved in dichloromethane (30 mL), and di-t-butyl dicarbonate (3.49 g, 16.0 mmol) was added. The solution was stirred for 6 h, then diluted with dichloromethane (100 mL), washed with saturated sodium bicarbonate (50 mL) and brine (50 mL), dried over magnesium sulfate, and concentrated. The crude was purified by flash column chromatography (25% ethyl acetate/hexane) to give compound 1-1d as a viscous, pale yellow oil (6.60 g, 82% over 2 steps). MS=536.1 ((M+H)⁺).

Step 1C: Deprotection, (2-Piperazin-1-yl-benzyl)-(2-thiophen-2-yl-ethyl)-carbamic acid tert-butyl ester, 1-1e

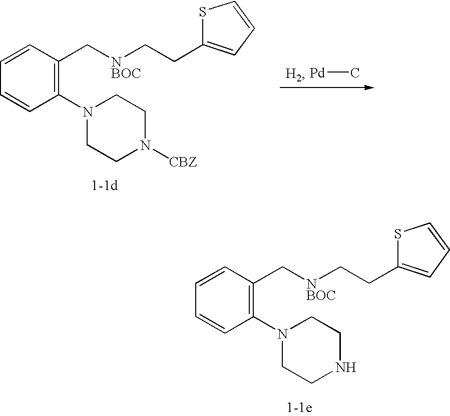

A mixture of 1-1d (6.30 g, 11.8 mmol) and 10% Pd/C (650 mg) in 80 mL ammonial methanol (7 M) was hydrogenated in a Parr apparatus at 40 PSI for 1 h. A second batch of catalyst (650 mg) was added, and the mixture was hydrogenated for 4 h. A third batch of catalyst (650 mg) was added and the mixture was hydrogenated for 18 hours, then filtered through Celite, concentrated in vacuo, and purified by flash column chromatography. Remaining starting material was eluted first (50% ethyl acetate/hexane) followed by the title compound 1-1e as a viscous, pale yellow oil (10% methanol/dichloromethane) (1.65 g, 35%). MS=402.0 ((M+H)⁺)

Step 1D: Peptide Coupling and Deprotection, R-(2-{4-[2-Amino-3-(4-chlorophenyl)-propionyl]-piperazin-1-yl}-benzyl)-(2-thiophen-2-yl-ethyl)-carbamic acid t-butyl ester, 1-1f

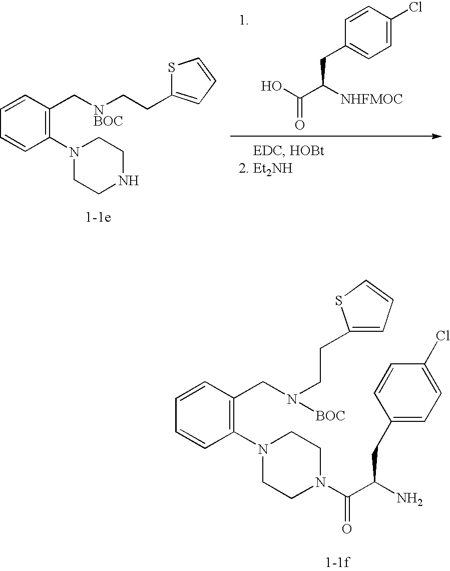

To a mixture of 1-1e (880 mg, 2.19 mmol) and (D)-N—FMOC-(4-chlorophenyl)alanine (1020 mg, 2.41 mmol) in dichloromethane (30 mL) was added HOBT (325 mg, 2.41 mmol), and the mixture was stirred for 20 min. EDC (460 mg,2.41 mmol) was added, and stirring was continued for 18 more hours. The mixture was then washed with saturated sodium bicarbonate (2×15 mL) and brine (15 mL), dried over magnesium sulfate, and concentrated in vacuo. The crude was filtered through silica gel (10% ethyl acetate/dichloromethane), concentrated, and dissolved in a 1:1 mixture of diethylamine:dichloromethane (20 mL). After stirring 3 h, the solution was concentrated, and isolated by flash column chromatography (9:1 ethyl acetate:dichloromethane to 94:5:1 dichloromethane:methanol:triethylamine) to give 1-1f as a white foam (1.11 g, 87% over 2 steps). MS=583.2 ((M+H)⁺)

Step 1E: Peptide Coupling and Deprotection, R-3-Amino-N-[1-(4-chlorobenzyl)-2-oxo-2-(4-{2-[(2-thiophen-2-yl-ethylamino)-methyl]-phenyl}-piperazin-1-yl)-ethyl]-propionamide, 1-1

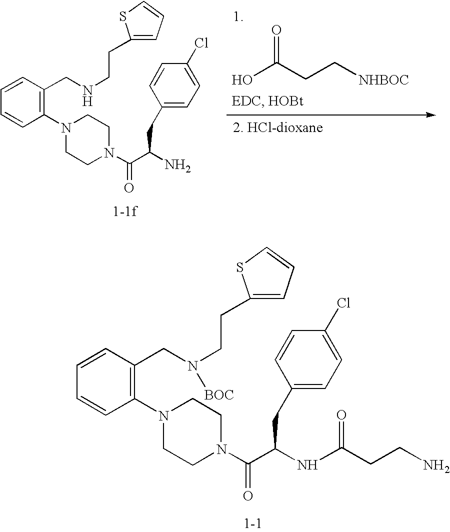

To a mixture of 1-1f (30 mg, 0.052 mmol) and N—BOC-β-alanine (11 mg, 0.058 mmol) in dichloromethane (0.5 mL) was added HOBT (8 mg, 0.06 mmol), and the mixture was stirred for 10 min. EDC (11 mg, 0.057 mmol) was added, and stirring was continued overnight. The mixture was washed with saturated sodium bicarbonate (1 mL), and separated. The aqueous layer was extracted with ethyl acetate (1 mL), and the combined organics were dried over sodium sulfate and concentrated. 4 M HCl/dioxane (1 mL) was added, and the mixture was stirred for 2 h, then concentrated and purified by HPLC to give the title product 1-1 (TFA salt) as a white solid. MS=554.2 ((M+H)⁺).

Other compounds were prepared from 1-1f using the same procedure shown in step 1E.

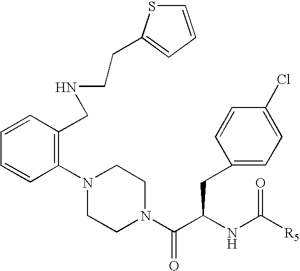

| Cpd | —R₅ | Mol Wt | MS ION | Reten Time |
|---|---|---|---|---|
| 1-1 | 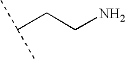 | 554.2 | 554.2 | 2.26 |
| 1-2 | 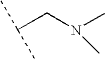 | 568.2 | 568.2 | 2.28 |
| 1-3 | 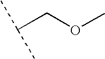 | 555.1 | 555.2 | 2.44 |
| 1-4 | 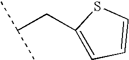 | 607.2 | 607.2 | 2.55 |
| 1-5 | 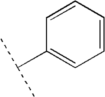 | 588.2 | 588.2 | 2.38 |
| 1-6 | 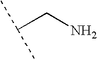 | 540.1 | 540.2 | 2.25 |
| 1-7 | 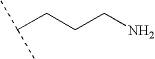 | 568.2 | 568.2 | 2.27 |
| 1-8 | 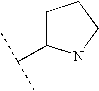 | 580.2 | 580.2 | 2.3 |
| 1-9 | 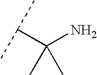 | 568.2 | 568.2 | 2.27 |
| 1-10 | 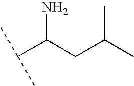 | 596.2 | 596.3 | 2.39 |
| 1-11 | 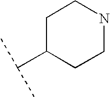 | 594.2 | 594.2 | 2.29 |
| 1-12 | 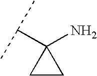 | 566.2 | 566.2 | 2.3 |
| 1-13 | 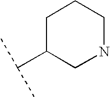 | 594.2 | 594.2 | 2.28 |
| 1-14 | 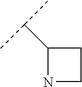 | 566.2 | 566.2 | 2.28 |
| 1-15 | 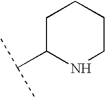 | 594.2 | 594.2 | 2.31 |
| 1-16 | 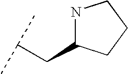 | 594.2 | 594.2 | 2.3 |

### Example 2

### 1,2,3,4-TETRAHYDRO-ISOQUINOLINE-3-CARBOXYLIC ACID [2-[4-(2-{[BENZYL-(2-DIMETHYLAMINO-ETHYL)-AMINO]-METHYL}-PHENYL)-[1,4]DIAZEPAN-1-YL]-1-(4-CHLORO-BENZYL)-2-OXO-ETHYL]-AMIDE (AS MONOTRIFLUOROACETATE)

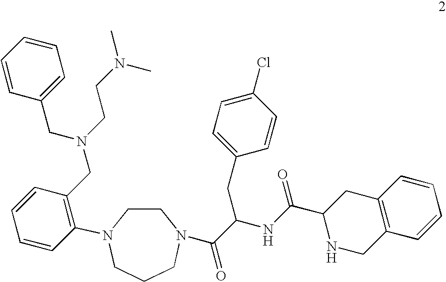

Step 2A: N-benzyl homopiperazine, 4-(2-formyl-phenyl)-[1,4]diazepane-1-carboxylic acid tert-butyl ester, 2-1a

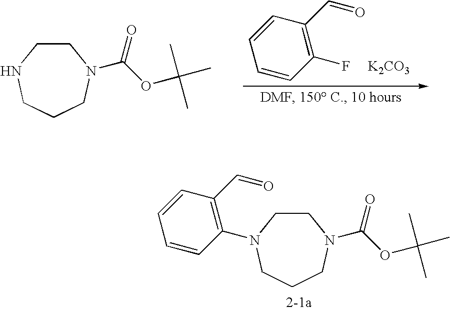

N-t-BOC-homopiperazine (12.02 g, 60 mmol), 2-fluorobenzaldehyde (7.45 g, 60 mmol) and potassium carbonate (12.44 g, 90 mmol) in 120 mL of DMF were heated to 150° C. for 10 hours. Upon cooling, the reaction mixture was treated with water (2×100 mL), extracted with ethyl acetate (3×100 mL) and purified by silica column chromatography (hexanes/ethyl acetate 1:1) to yield compound 2-1a (12.04 g, 66%).

Step 2B: Deprotection and Purification, 2-1b

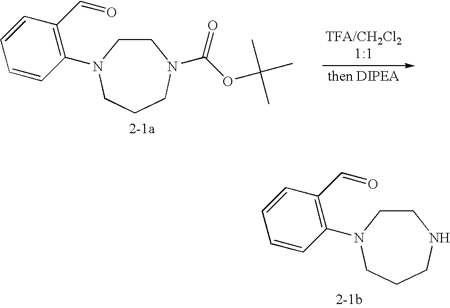

Compound 2-1a (304.3 mg, 1 mmol) was dissolved in mixture of methylene chloride/trifluoroacetic acid (2 mL/2 mL) and was stirred vigorously for 30 minutes at room temperature. Solvents were evaporated and the residue was dissolved in 5 mL methylene chloride. 3 mL diisopropylethylamine was added and evaporation under vacuum gave 2-1b.

Step 2C: Preparation of the dipeptide 2-1c

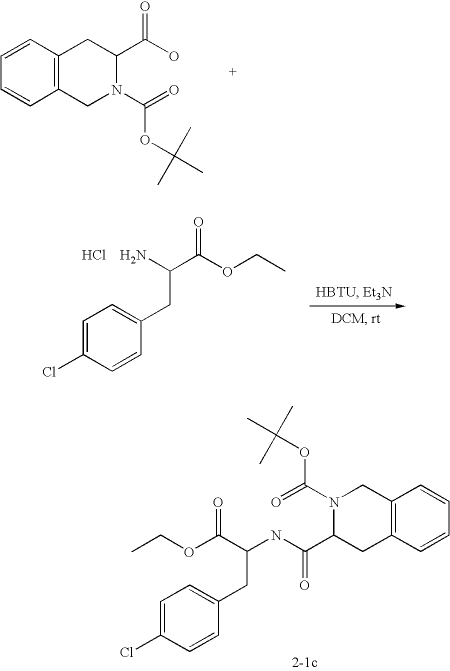

D,L-4-chlorophenylalanine ethyl ester hydrochloride (10.0 g, 37.8 mmol) and N—BOC-1,2,3,4-tetrahydroisoquinoline-3-carboxylic acid (10.47 g, 37.8 mmol) were dissolved in methylene chloride (100 mL). HBTU (21.5 g, 56.79 mmol) and triethylamine (11 mL, 75.72 mmol) were added and the reaction mixture was stirred overnight at room temperature. The reaction mixture was washed with aqueous sodium bicarbonate (3×25 mL) and aqueous sodium chloride solution (25 mL). The organic layer was collected, dried over anhydrous NaSO₄, filtered and solvent removed in vacuo. The resulting residue was purified by column chromatography to give 2-1c.

Step 2D: Saponification Step, 2-1d

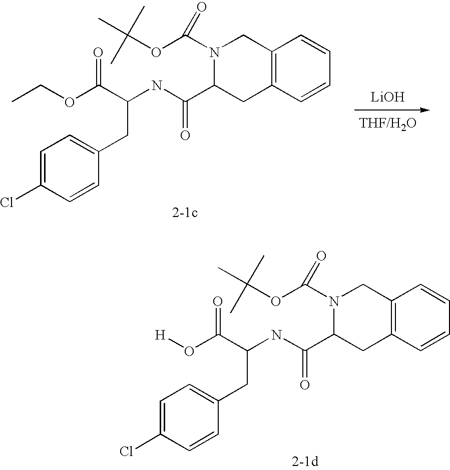

Compound 2-1c (4.86 g, 10 mmol) was dissolved in a mixture of methanol/tetrahydrofuran (10 mL/10 mL). 10 mL of a 2N solution of lithium hydroxide in water was added. The solution was stirred for 18 hours at room temperature. Solvents were removed, 100 mL of water was added to the residue. The aqueous layer was extracted with diethyl ether (2×30 mL). The aqueous layer was acidified with acetic acid and then extracted with ethyl acetate, dried over magnesium sulfate, filtered and solvent removed in vacuo. The acid 2-1d was obtained with 68% yield.

Step 2E: Coupling of dipeptide 2-1d, 2-3,3-{1-(4-chloro-benzyl)-2-[4-(2-formyl-phenyl)-[1,4diazepan-1-yl]-2-oxo-ethylcarbamoyl}-3,4-dihydro-1H-isoquinoline-2-carboxylic acid tert-butyl ester, 2-1e

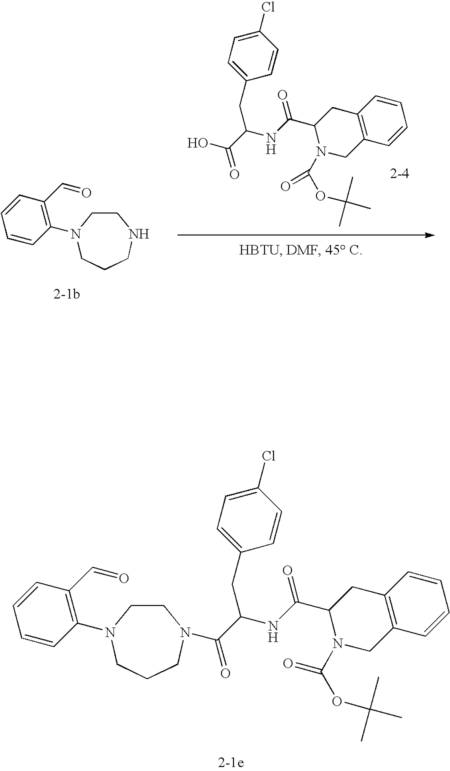

2-1b (204 mg, 1 mmol) dissolved in 2 mL of DMF was added to a mixture of dipeptide 2-1d (459 mg, 1 mmol) and HBTU (457 mg, 1.2 mmol), previously stirred in 4 mL of DMF for 30 minutes at 40° C. The mixture was stirred at 40° C. for 6 additional hours. Water (5 mL) was added, the product was extracted with diethyl ether and purified on silica (hexanes/ethyl acetate 1:1). The yield of the compound 2-1e was 415 mg (64%).

Step 2F: Reductive Amination and Deprotection, 1,2,3,4-Tetrahydro-isoquinoline-3-carboxylic acid [2-[4-(2-{[benzyl-(2-dimethylamino-ethyl)-amino]-methyl}-phenyl)-[1,4diazepan-1-yl]-1-(4-chloro-benzyl)-2-oxo-ethyl]-amide (as monotrifluoroacetate), 2-1

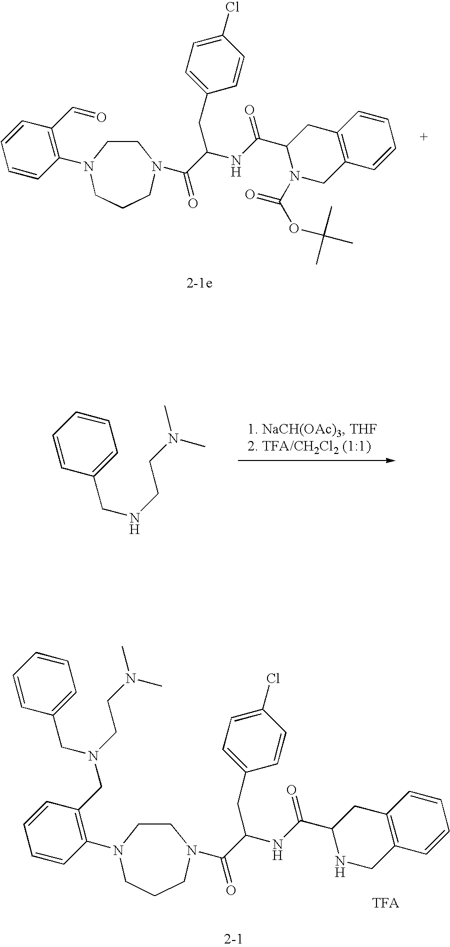

Aldehyde 2-1e (38.7 mg, 60 μmol) and N′-benzyl-N,N-dimethyl-ethane-1,2-diamine (25.7 mg, 144 μmol) in 500 μL of THF were stirred at RT for 30 minutes. Sodium triacetoxyborohydride (25.4 mg, 120 μmol) was added and stirring continued for 6 hours at room temperature. Water was added followed by extraction with ethyl acetate and purification by preparative HPLC. The BOC group was removed by 30 minutes treatment with TFA/CH₂Cl₂ (1:1) to give product 2-1.

Other examples were prepared from 2-1 e using the same procedure shown in step 2-F.

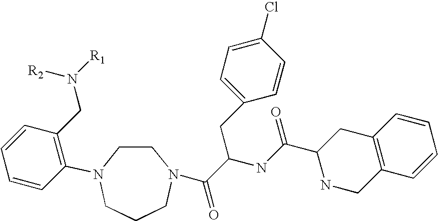

| Cpd | —NR₁R₂ | Mol Wt | MS ION | Retin Time |
|---|---|---|---|---|
| 2-1 | 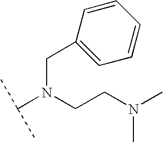 | 707.4 | 707.2 | 2.374 |
| 2-2 | 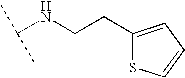 | 656.3 | 656.1 | 2.338 |
| 2-3 | 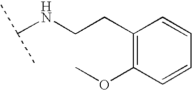 | 680.3 | 680.1 | 2.455 |
| 2-4 | 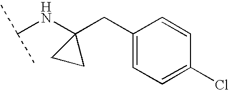 | 710.7 | 710.1 | 2.519 |
| 2-5 | 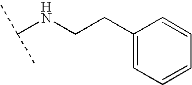 | 650.3 | 650.1 | 2.431 |
| 2-6 | 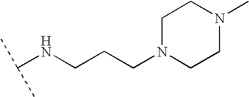 | 686.3 | 686.2 | 2.21 |

### Example 3

### 1,2,3,4-TETRAHYDRO-ISOQUINOLINE-3-CARBOXYLIC ACID [2-[1-(2-THIOPHEN-2-YL-ETHYLAMINO)ETHYL}-PHENYL)-PIPERAZIN-1-YL]-1-(4-CHLORO-BENZYL)-2-OXO-ETHYL]-AMIDE (AS MONOTRIFLUOROACETATE)

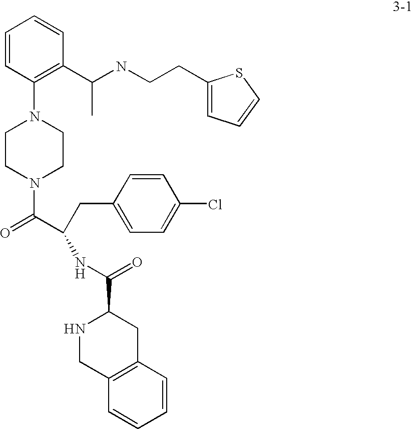

Step 3A: Addition of the 2-fluoroacetophenone to N-Boc piperazine, 3-1a

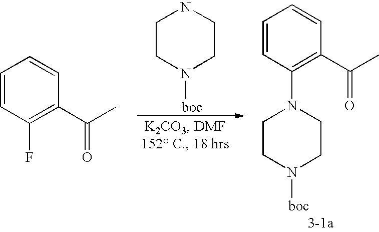

N-Boc-piperazine (20.0 g, 108 mmol) and 2-fluoroacetophenone (13 g, 108 mmol) were suspended in DMF (108 mL) and treated with potassium carbonate (22 g, 161 mmol). The reaction mixture was heated at 152° C. for 18 h. The mixture was then cooled, dissolved in ethyl acetate (100 mL), washed with water (100 mL) and aqueous NaCl (3×10 mL), dried over anhydrous MgSO₄, filtered, and the solvent removed in vacuo. The residue was diluted with hexane (200 mL) and filtered. The solvent was discarded and the residue was collected and dried under vacuum to afford 22 g (71%) of 3-1a as a yellow solid. MS=290 (M+H)⁺

Step 3B: Deprotection and Acid Coupling, 3-1c

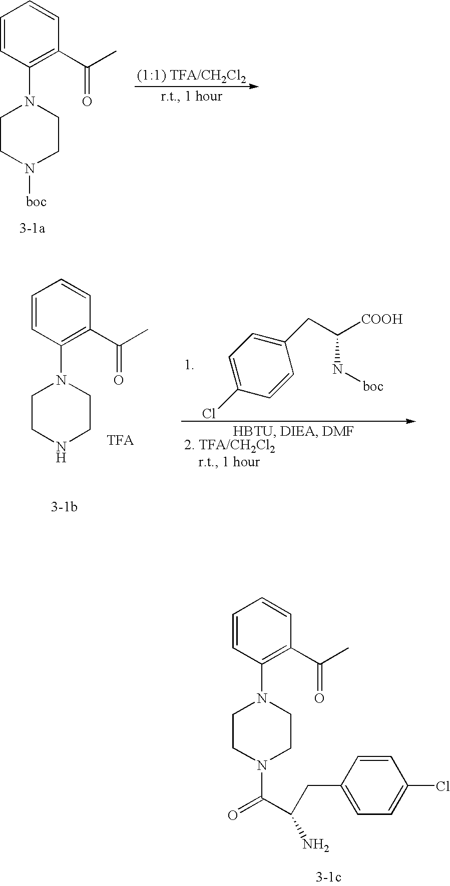

Compound 3-1a (1.50 g, 5 mmol), was dissolved in dichloromethane (10 mL) and was treated with TFA (10 mL). The mixture stirred for 1 h under a nitrogen atomosphere. Solvent was removed in vacuo, the residue was diluted with dichloromethane and concentrated in vacuo (dilution done four times) to afford the TFA salt of 3-1b as a tan solid in quantitative yield. MS=247 ((M+H)⁺)

Boc-d-4-chlorophenylalanine (5.00 g, 16.72 mmol) was dissolved in DMF (35 mL), treated with diisopropylamine (6.90 g, 53.76 mmol) and HBTU (6.30 g, 16.72 mmol). The mixture stirred at room temperature for 1 h under a nitrogen atmosphere. Compound 3-1b (3.40 g, 16.72 mmol) was added and the mixture stirred at room temperature for 18 h. The mixture was diluted with ethyl acetate (50 mL) and washed with aqueous sodium bicarbonate (3×25 mL) and aqueous sodium chloride solution (25 mL). The organic layer was collected, dried over anhydrous NaSO₄, filtered and solvent removed in vacuo. The resulting residue was purified by column chromatography on silica using 50% ethyl acetate/hexanes as the eluent to afford the BOC protected material (6.50 g, 85%) as a light yellow solid. MS=486 (M+H)⁺. The resulting protected material was suspended in dichloromethane (10 mL) and treated with TFA (10 mL). The mixture stirred for 1 h under a nitrogen atmosphere. Solvent was removed in vacuo, then the residue was diluted with dichloromethane (75 mL) and washed with aqueous sodium bicarbonate (3×25 mL) and aqueous sodium chloride (25 mL). The organic layer was extracted, dried over anhydrous NaSO₄, filtered, and concentrated in vacuo to afford compound 3-1c (1.50 g, 80% yield) as a light tan solid. MS=386 ((M+H)⁺)

Step 3C: Coupling, 3-1d

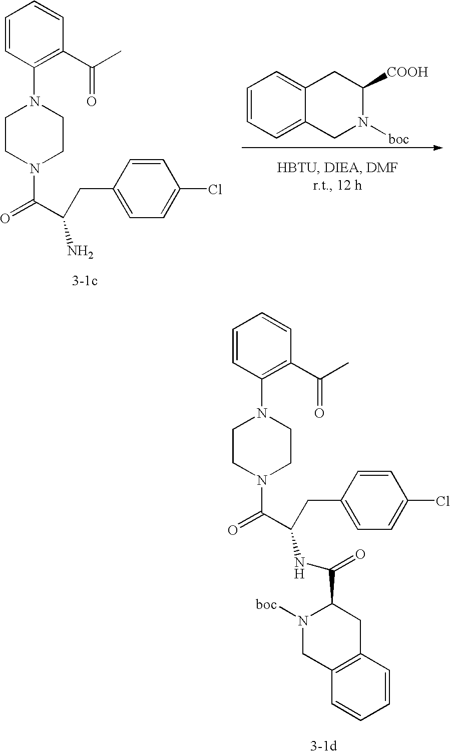

N-BOC-1,2,3,4-tetrahydroisoquinoline-3-carboxylic acid (1.00 g, 3.88 mmol) was dissolved in DMF (8 mL) and treated with diisopropylamine (0.995 g, 7.72 mmol) and HBTU (1.50 g, 3.88 mmol). The mixture stirred for 1 h under a nitrogen atmosphere followed by addition of compound 3-1c (1.50 g, 3.88 mmol). The mixture continued to stir for 18 h. The mixture was diluted with ethyl acetate (50 mL) and washed with aqueous sodium bicarbonate (3×25 mL) and aqueous sodium chloride solution (25 mL). The organic layer was collected, dried over NaSO₄, filtered and solvent removed in vacuo. The resulting residue was purified by column chromatography on silica using 50% ethyl acetate/hexanes as the eluent to afford the 3-1d as a light tan solid (2.10 g, 84%). MS=645 ((M+H)⁺)

Step 3D: Reductive Amination, 3-1

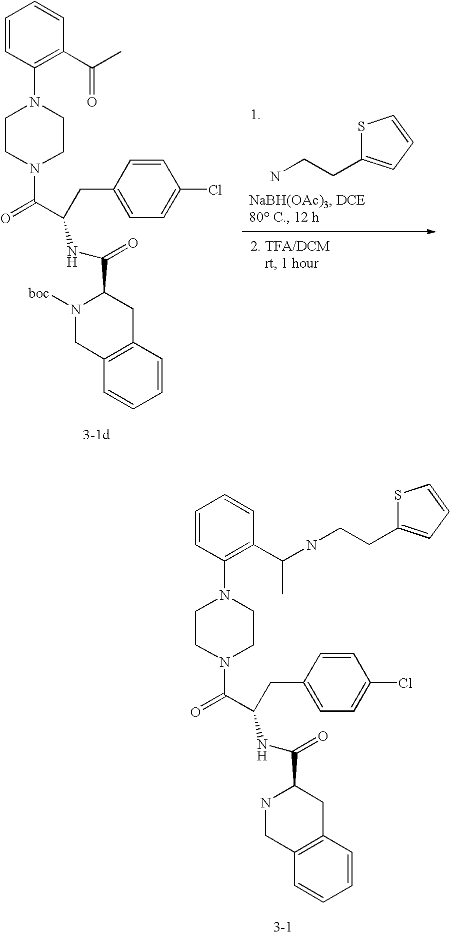

A 0.2 M stock solution of compound 3-1d (0.129 g, 0.2 mmol) was prepared in dichloroethane and added to 2-thiophen-2-yl-ethylamine (0.3 mmol). The mixture was treated with acetic acid (0.012 mL, 0.2 mmol) and stirred for 1 h. Sodium triacetoxyborohydride (0.06 g, 0.280 mmol) was added and the mixture stirred for 12 h at 80° C. The mixture was allowed to cool to room temperature. Solvent was removed under a stream of nitrogen. The residue was resuspended in dichloromethane (1 mL) and washed with aqueous sodium bicarbonate solution (1 mL). The organic layer was extracted and solvent was removed under a stream of nitrogen. The crude product was then deprotected by suspending it in dichloromethane (1 mL) and treated with TFA (1 mL). The mixture stirred in capped vials for 1 h. Solvent was removed by a stream of nitrogen. The residue was purified by preparative HPLC to afford 3-1 as a pure compound.

Other compounds were prepared from 3-1c using the same procedure shown in steps 3C and 3D.

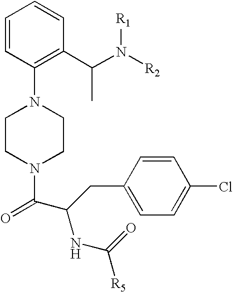

| Cpd | —NR₁R₂ | —R₅ | Mol Wt | MS ION | Reten Time |
|---|---|---|---|---|---|
| 3-1 | 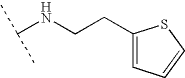 | 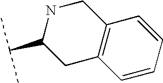 | 656.3 | 656 | 2.403 |
| 3-2 | 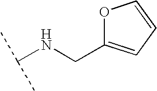 | 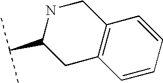 | 626.2 | 626 | 2.327 |
| 3-3 | 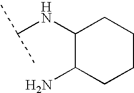 | 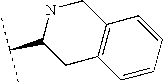 | 643.3 | 643 | 2.187 |
| 3-4 | 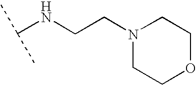 | 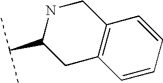 | 659.3 | 659 | 2.203 |
| 3-5 | 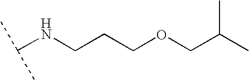 | 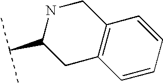 | 660.3 | 660 | 2.455 |
| 3-6 | 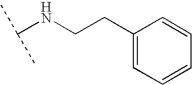 | 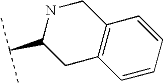 | 650.3 | 650 | 2.415 |
| 3-7 | 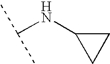 | 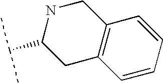 | 586.2 | 586 | 2.295 |
| 3-8 | 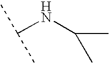 | 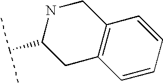 | 588.2 | 588 | 2.309 |
| 3-9 | 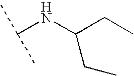 | 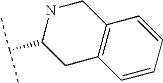 | 616.2 | 616 | 2.371 |
| 3-10 | 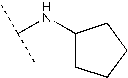 | 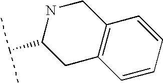 | 614.2 | 614 | 2.347 |
| 3-11 | 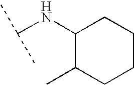 | 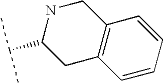 | 642.3 | 642 | 4.38 |
| 3-12 | 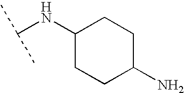 | 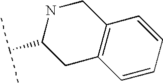 | 643.3 | 643 | 3.56 |
| 3-13 | 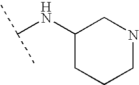 | 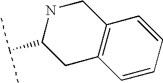 | 629.2 | 629 | 3.54 |
| 3-14 | 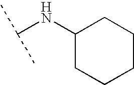 | 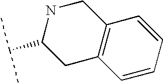 | 628.3 | 628 | 4.25 |
| 3-15 | 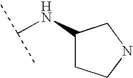 | 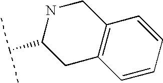 | 615.2 | 615 | 3.61 |
| 3-16 | 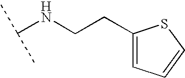 | 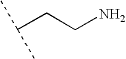 | 568.2 | 568 | 4.14 |
| 3-17 | 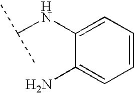 | 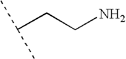 | 549.1 | 549 | 3.97 |
| 3-18 | 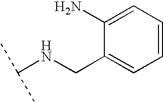 | 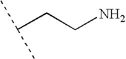 | 563.1 | 563 | 3.93 |
| 3-19 | 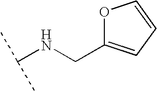 | 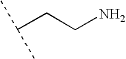 | 538.1 | 538 | 3.81 |
| 3-20 | 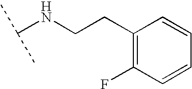 | 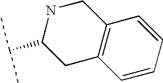 | 668.3 | 668 | 2.412 |

### Example 4

### R-3-AMINO-N-[1-(4-CHLOROBENZYL)-2-OXO-2-(4-{2-[1-(2-THIOPHEN-2-YL-ETHYLAMINO)-ETHYL]-PYRIDINYL}-PIPERAZIN-1-YL)-ETHYL]-PROPIONAMIDE

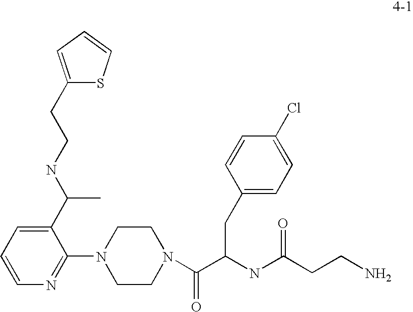

Step 4A: 2-Chloro 3-acetylpyridine, 4-1a

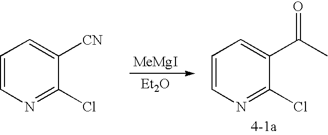

2-Chloro-3-cyanopyridine (1 g, 7.24 mmol) was dissolved in diethyl ether (50 mL) and was cooled to −78° C. under nitrogen. A solution of methyl magnesium iodide (3M in diethyl ether) was slowly added over 10 minutes. The reaction was removed from the ice bath and stirred at ambient temperature for 5 hours. It was then cooled to 0° C. and quenched with 1M HCl until acidic (pH=2). Following extraction with diethyl ether (3×30 mL), the organic layers were combined and washed with water (30 mL), brine (30 mL) and dried over sodium sulfate. The solution was concentrated in vacuo to afford 4-1a as an oil in quantitative yield. MS=155 ((M+H)⁺)

Step 4B: N-(3-Acetylpyridyl)piperazine, 4-1b

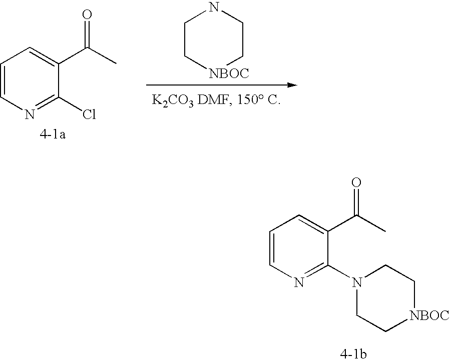

Ketone 4-1a (1.2 g, 7.7 mmol), boc-piperazine (1.4 g, 7.7 mmol) and potassium carbonate (1.4 g, 10 mmol) were dissolved in DMF (15 mL) and refluxed at 150° C. for 2 hours. The reaction was cooled, diluted with ethyl acetate (60 mL), washed with water (3×20 mL) and brine (20 mL), dried over sodium sulfate, and concentrated. The residue was purified by silica gel chromatography (elution with 20% ethyl acetate in hexanes) to afford 1.18 g (51%) of 4-1b as a clear oil. MS=305 ((M+H)⁺)

Step 4C: Deprotection and Peptide Coupling, 4-1c

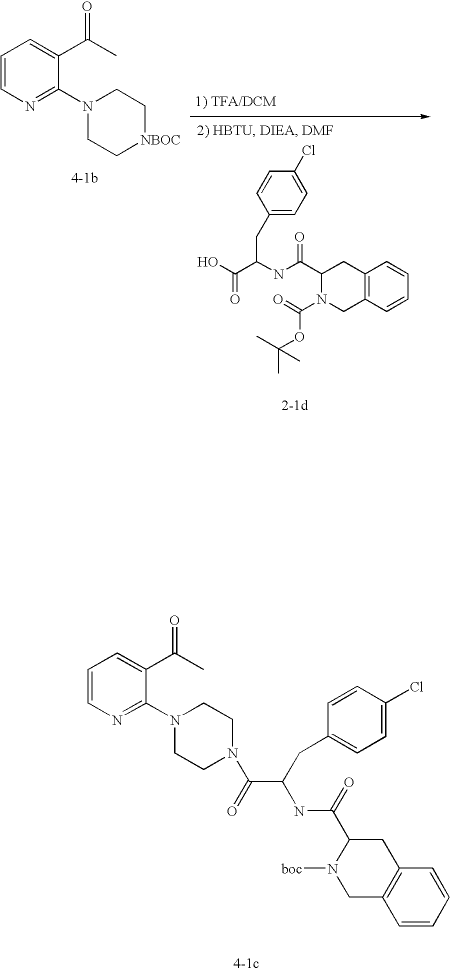

Dipeptide 2-1d (0.409 g, 1.2 mmol) was dissolved in DMF (8 mL) and treated with diisopropylamine (0.309 g, 2.4 mmol) and HBTU (1.50 g, 1.2 mmol). The mixture stirred for 1 h under a nitrogen atmosphere. Compound 4-1b (0.46 g, 1.2 mmol) was added and the mixture continued to stir for 18 h. The mixture was diluted with ethyl acetate (50 mL) and washed with aqueous sodium bicarbonate (3×25 mL) and aqueous sodium chloride solution (25 mL). The organic layer was collected, dried over NaSO₄, filtered and solvent removed in vacuo. The resulting residue was purified by column chromatography on silica using 50% ethyl acetate/hexanes as the eluent to afford 4-1c as a light tan solid (yield: 84%).

Step 4D: Reductive Amination and Deprotection, 4-1

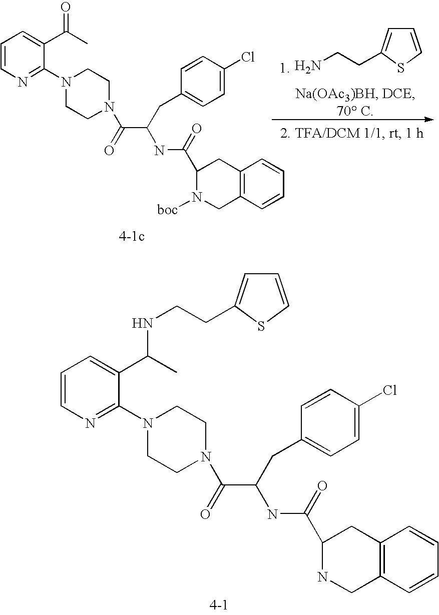

A portion of ketone 4-1c (350 mg, 1.1 mmol) and 2-thiopheneethylamine (137 mg, 1.1 mmol) were dissolved in 1,2-dichloroethane (5 ml) and stirred for 10 minutes. Sodium triacetoxyborohydride (370 mg, 1.7 mmol) was then added and the reaction was stirred overnight at 70° C. The reaction was cooled, diluted with dichloromethane (10 mL), washed with 10% sodium bicarbonate (10 mL) and brine (10 mL), dried over sodium sulfate and concentrated. A portion of the residue (50 mg) was dissolved in methanol (1 mL) and purified via HPLC-MS. MS=568 ((M+H)⁺). This material was dissolved in 1 mL CH₂Cl₂ and was treated with 1 mL anhydrous TFA, after 30 minutes the solvent was removed in vacuo to give the deprotected product 4-1.

### Example 5

### R-3-AMINO-N-[1-(4-CHLOROBENZYL)-2-OXO-2-(4-{2-[(2-(2-METHOXY) PHENETHYLAMINO)-METHYL]3-FLUOROPHENYL}-PIPERAZIN-1-YL)-ETHYL]-PROPIONAMIDE

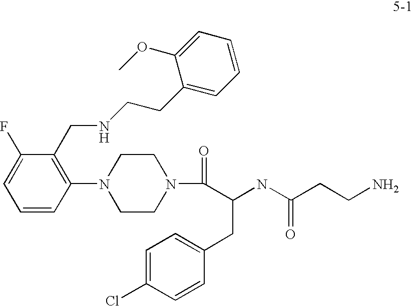

Step 5A: Preparation of Peptide 5-1a

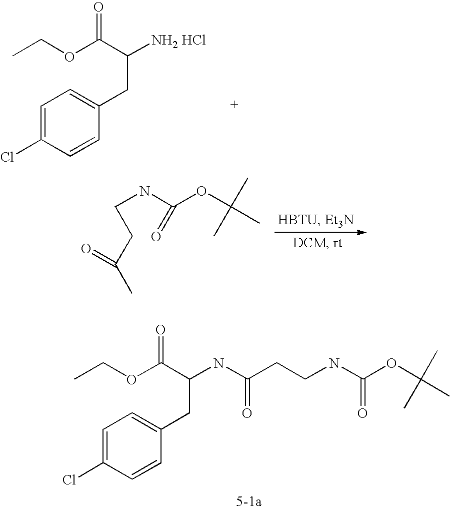

D,L-4-chlorophenylalanine ethyl ester hydrochloride (10.0 g, 37.8 mmol) and N-BOC-beta-alanine (7.16 g, 37.8 mmol) were dissolved in 100 mL methylene chloride. HBTU (21.5 g, 56.79 mmol) and triethylamine (11 mL, 75.72 mmol) were added. The reaction mixture was stirred overnight at room temperature. The reaction mixture was washed with aqueous sodium bicarbonate (3×25 mL) and aqueous sodium chloride solution (25 mL). The organic layer was collected, dried over anhydrous NaSO₄, filtered and solvent removed in vacuo. The resulting residue was purified by column chromatography to give 13.19 g of 5-1a (Yield 87%).

Step 5B: Saponification of 5-1a, 5-1b

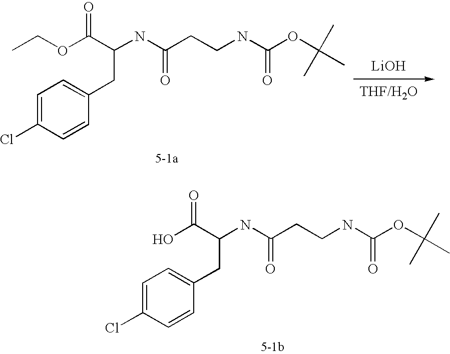

Compound 5-1a (13.19 g, 33.06 mmol) was dissolved in a mixture of methanol/tetrahydrofuran (20 mL/20 mL). 30 mL of a 2N solution of lithium hydroxide in water was added. The solution was stirred for 18 hours at room temperature. Solvents were removed and 100 mL of water was added to the residue. The aqueous layer was extracted with diethyl ether (2×30 mL). The aqueous layer was acidified with acetic acid and then extracted with ethyl acetate, dried over magnesium sulfate, filtered and solvent removed in vacuo. The acid 5-1b was obtained with 66% yield.

Step 5C: Piperazine Coupling, 5-1c

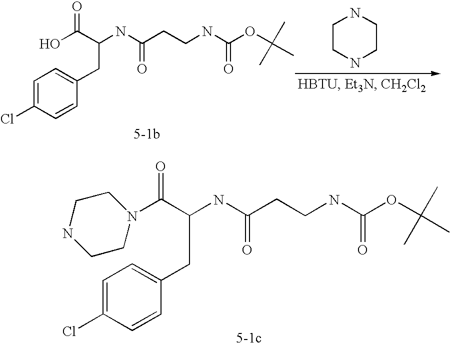

Compound 5-1b (9.12 g, 24.59 mmol) was dissolved in 75 mL of CH₂Cl₂. HBTU (14 g, 36.89 mmol) and triethylamine (7 mL, 49.18 mmol) were added. The mixture was stirred for 30 minutes, piperazine (4.24 g, 49.18 mmol) was added and the solution stirred at room temperature for 18 hours. The reaction mixture was washed with an aqueous solution of citric acid (50 mL), a saturated solution of bicarbonate (50 mL), and brine (100 mL). The mixture was dried over magnesium sulfate, filtered, and the solvent removed in vacuo. The residue was purified on silica to give compound 5-1c (10.11 g, Yield 94%).

Step 5D: Addition of 2,6-difluorobenzaldehyde, 5-1d

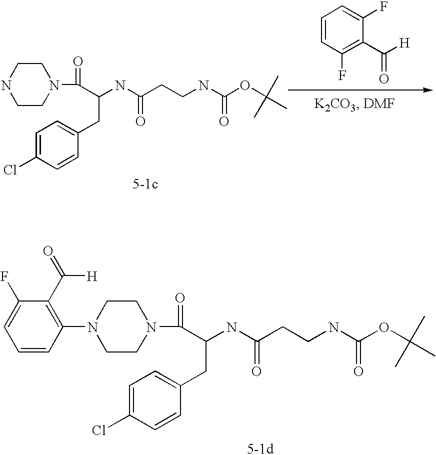

Compound 5-1c (0.2 mg, 0.45 mmol) and 2,6-difluorobenzaldehyde were heated in 2 mL DMF with 75 mg of potassium carbonate (0.55 mmol) for 18 hours at 90° C. After filtration of the reaction mixture, the solvent was evaporated and 5 mL of water were added. The product was extracted with ethyl acetate and purified by silica gel liquid chromatography to give compound 5-1d (55% yield).

Step 5E: Reductive Amination, 5-1

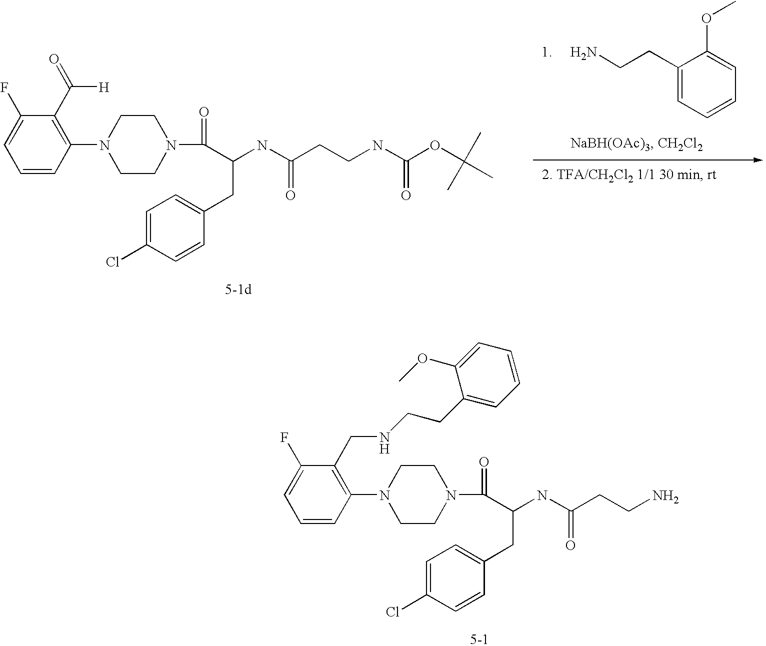

Aldehyde 5-1d (100 mg, 0.17 mmol) and 2-methoxyphenethylamine (26 μl, 0.17 mmol) were dissolved in dichloromethane (1 mL) and were stirred for 1 hour. Sodium triacetoxyborohydride (75 mg, 0.35 mmol) was then added and the reaction was stirred overnight at room temperature. The reaction was filtered and the solvent was evaporated. Methanol (1 mL) was added to the residue, which was then purified by reverse phase HPLC. MS=696 ((M+H)⁺). This material was dissolved in 1 mL CH₂Cl₂ and was treated with 1 mL anhydrous TFA for 30 minutes and the solvent was removed in vacuo to give the deprotected product 5-1.

### Example 6

### 1-[2-(2-AMINOPROPIONYLAMIDO)-(3R)-(2,4-DICHLOROPHENYL)PROPIONYLI-4-[(2R, S)-(2′-FLUOROBENZYLAMINOPROPYLIPHENYLPIPERAZINE

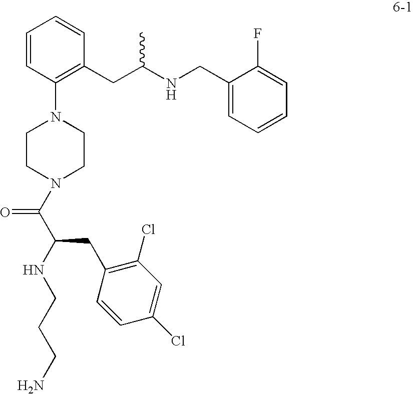

Step 6A: 2-[4-(t-Butoxycarbonyl)piperazin-1-yl]benzaldehyde 6-1a

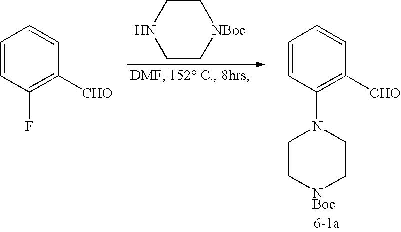

A mixture of 2-fluorobenzaldehyde (8.54 mL, 80.54 mmol), 1-(t-butoxycarbonyl)-piperazine (15 g, 80.54 mmol), and potassium carbonate (16.75 g. 121.16 mmol) in DMF (81 mL) was heated at 150° C. for 8 hours with constant stirring. The reaction mixture was cooled to room temperature, diluted with ethyl acetate (200 mL), and washed with water (3×150 mL) and saturated NaCl solution (150 mL). The organic layer was dried over anhydrous MgSO₄, filtered, and concentrated in vacuo. The yellow oil solidified under vacuum overnight giving a bright yellow solid. The solid was washed with hexanes (3×100 mL) to removed impurities, collected and dried under high vacuum. Compound 6-1a was obtained as a bright yellow solid in 75% yield (17.5 g).

Step 6B: 1-(tert-Butoxycarbonyl)-4-[2-(2-nitrovinyl)phenylpiperazine 6-1b

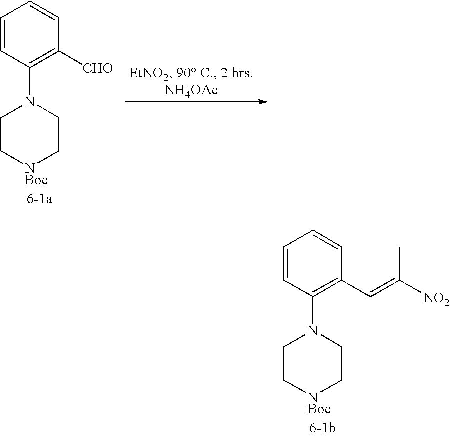

A mixture of 2-[4-(t-butoxycarbonyl)piperazin-1-yl]benzaldehyde 6-1a (7 g, 24.1 mmol), nitroethane (48 mL, 667.6 mmol), and ammonium acetate (0.84 g. 10.85 mmol) was heated for 2 hours at 90° C. with constant stirring under a nitrogen atmosphere. The mixture was cooled to room temperature and excess nitroethane was removed under vacuum. The residual yellow oil was diluted with ethyl acetate (150 mL), washed with water (3×100 mL), and saturated NaCl solution (100 mL). Solvent was removed in vacuo and the crude product was purified by column chromatography on silica using 100% dichloromethane as the eluent (R_{f}=0.3). Compound 6-1b was obtained in 63% yield (5.3 g) as a yellow solid.

Step 6C: 1-(tert-Butoxycarbonyl)-4-[2-(acetonyl)phenylpiperazine 6-1c

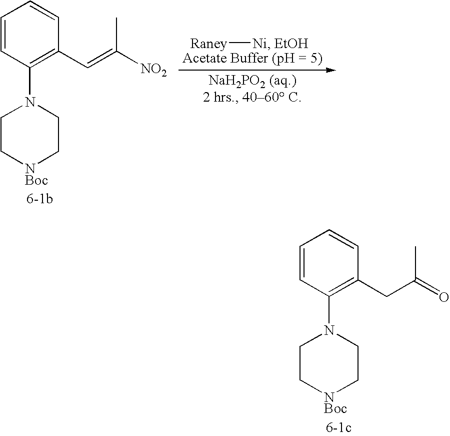

1-(tert-Butoxycarbonyl)-4-[2-(2-nitrovinyl)phenylpiperazine 6-1b (5.3 g, 15.3 mmol) was dissolved in ethanol (253 mL) and acetate buffer (pH=5, 77 mL). To the reaction mixture, Raney nickel (5 mL, Raney 2800 nickel, slurry in water) and NaH₂PO₄ solution (30 mL of 2.65M in water) were added simultaneously. After the addition was complete, the reaction was heated at 50° C. for 2 hours. The catalyst was then removed by filtration and washed with 50 mL of ethanol followed by 200 mL of water. The filtrate was extracted with ether (3×150 mL) and the organic layer was dried over anhydrous Na₂SO₄, filtered, and solvent removed in vacuo. The residual clear oil was purified by column chromatography on silica using 20% ethyl acetate/hexanes as the eluent (Rf=0.3). Compound 6-1c was recovered as a clear oil in 61% yield (2.99 g).

Step 6D: 1-[2-(2-tert-Butoxycarbonylaminopropionylamido)-3(R)-(2,4-dichlorophenyl)propionyl]-4-[2-(acetonyl)phenylpiperazine 6-1e

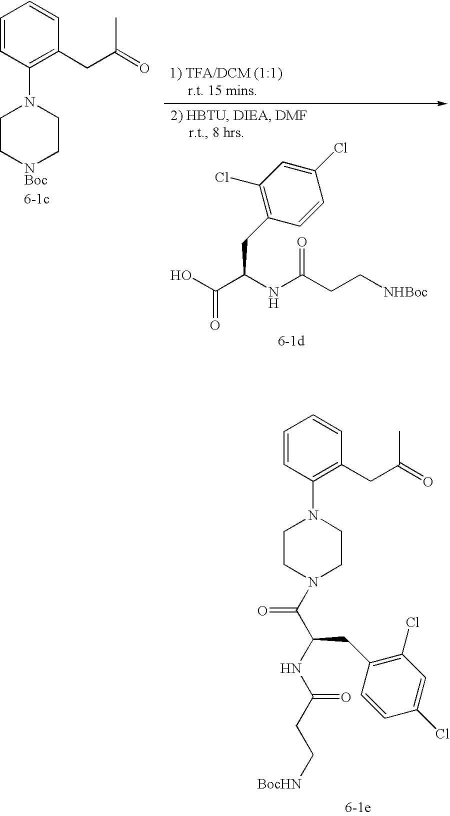

1-(tert-Butoxycarbonyl)-4-[2-(acetonyl)phenylpiperazine 6-1c. (1.44 g, 4.54 mmol) was dissolved in 8 mL of(1:1) trifluoroacetic acid/dicholormethane and stirred at room temperature for 20 minutes. The reaction mixture was evaporated to dryness, redissolved in dichloromethane (20 mL), and washed with saturated NaHCO₃ solution (3×20 mL). The organic layer was additionally washed with 20 mL of saturated NaCl solution, dried over anhydrous Na₂SO₄, filtered, and solvent removed in vacuo.

In a separate clean dried flask, N-(2-tert-butoxycarbonylaminopropionyl)-D-(2′,4′-dichloro)phenylalanine 6-1d (3 g, 9.4 mmol, prepared in a similar manner to Steps 5A and 5B) was dissolved in DMF (40 mL) along with diisopropylethyl amine (3.3 mL, 18.8 mmol) and HBTU (3.6, 9.4 mmol). The reaction mixture was allowed to stir at room temperature for 1 hour then 1-[2-(acetonyl)phenylpiperazine (prepared above) was added along with an additionally 6.5 mL of diisopropylethyl amine (37.6 mmol). The reaction was allowed to stir at room temperature for an additional 8 hours. The reaction mixture was then diluted with ethyl acetate (100 mL), and then was washed with water (3×100 mL), and saturated NaCl solution (100 mL). The organic layer was dried over anhydrous MgSO₄, filtered, and solvent removed in vacuo. Compound 6-1e was obtained as a brown solid in 80% yield (4.5 g) without further purification.

Step 6E: 1-[2-(2-Aminopropionylamido)-(3R)-(2,4-dichlorophenyl)propionyl]-4-[(2R,S)-(2′-fluorobenzylaminopropyl]phenylpiperazine 6-1

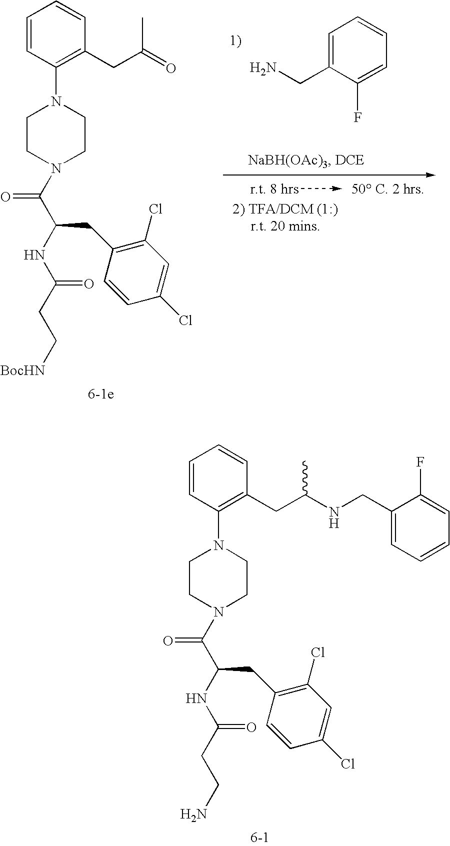

1-[2-(2-Aminopropionylamido)-(3R)-(2,4-dichlorophenyl)propionyl]-4-[2-(acetonyl)phenylpiperazine 6-1e (121 mg, 0.2 mmol) was dissolved in 1 mL of 1,2-dichloroethane. To the reaction vial, 2-fluorobenzyl amine (22.8 uL, 0.2 mmol) and glacial acetic acid (11.5 uL, 0.2 mmol) were added along with NaBH(OAc)₃ (59.3 mg, 0.28 mmol). The reaction mixture was allowed to stir for 8 hours at room temperature then quenched with 2 mL of 1N NaOH solution. The product was extracted with 2×5 mL of dichloromethane and the organic layer washed with 5 mL of saturated NaCl solution, dried over anhydrous MgSO₄, filtered, and solvent removed in vacuo. The residual oil was dissolved in 2 mL of (1:1) trifluoroacetic acid/dichloromethane and was stirred at room temperature for 20 minutes. The reaction mixture was then evaporated to dryness and the crude product was purified by preparative HPLC to give Compound 6-1. MS 615 (M⁺).

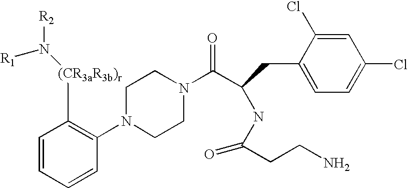

| Cpd | R₁R₂N(CR₃ₐR_{3b})ᵣ— | Mol Wt | MS ION | Reten Time |
|---|---|---|---|---|
| 6-1 | 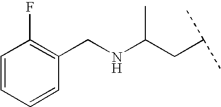 | 574.593 | 574 | 2.269 |
| 6-2 | 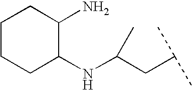 | 603.635 | 603 | 2.151 |
| 6-3 | 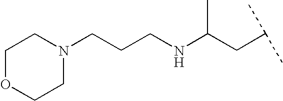 | 633.66 | 633 | 2.123 |
| 6-4 | 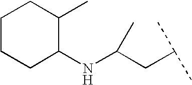 | 602.646 | 602 | 2.319 |
| 6-5 | 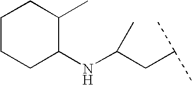 | 586.56 | 586 | 2.246 |
| 6-6 | 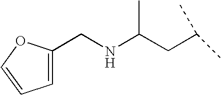 | 602.627 | 602 | 2.286 |
| 6-7 | 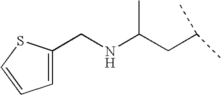 | 588.62 | 588 | 2.291 |
| 6-8 | 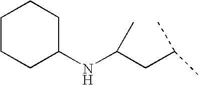 | 583.56 | 583 | 2.267 |
| 6-9 | 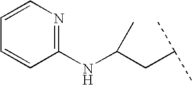 | 583.56 | 583 | 2.267 |
| 6-10 | 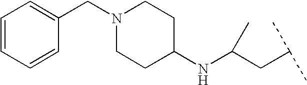 | 679.732 | 679 | 2.178 |
| 6-11 | 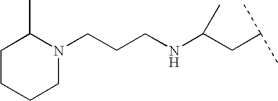 | 645.715 | 645 | 2.144 |
| 6-12 | 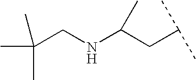 | 577.597 | 577 | 2.126 |
| 6-13 | 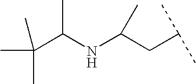 | 590.635 | 590 | 2.329 |
| 6-14 | 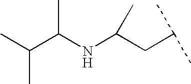 | 576.609 | 576 | 2.295 |
| 6-15 | 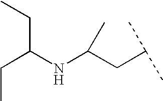 | 576.609 | 576 | 2.292 |
| 6-16 | 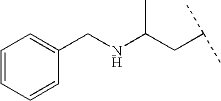 | 596.599 | 596 | 2.294 |
| 6-17 | 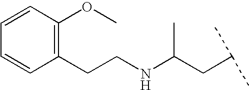 | 626.625 | 626 | 2.289 |
| 6-18 | 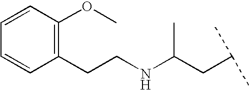 | 640.652 | 640 | 2.34 |
| 6-19 | 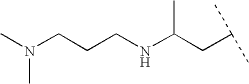 | 591.624 | 591 | 2.119 |
| 6-20 | 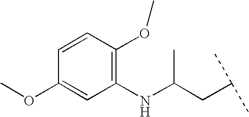 | 642.624 | 642 | 2.415 |
| 6-21 | 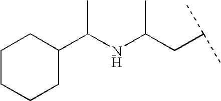 | 616.673 | 616 | 2.357 |
| 6-22 | 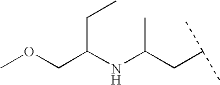 | 592.608 | 592 | 2.294 |
| 6-23 | 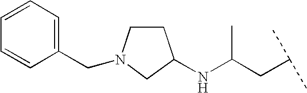 | 665.705 | 665 | 2.196 |
| 6-24 | 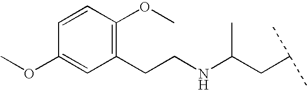 | 670.677 | 670 | 2.047 |
| 6-25 | 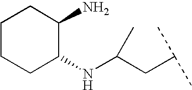 | 603.635 | 603 | 1.834 |
| 6-26 | 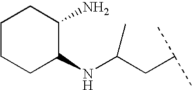 | 603.635 | 602 | 1.84 |
| 6-27 | 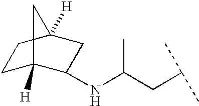 | 600.631 | 600 | 1.984 |
| 6-28 | 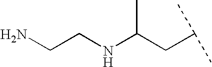 | 549.543 | 549 | 1.8 |
| 6-29 | 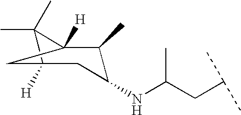 | 642.711 | 642 | 2.089 |
| 6-30 | 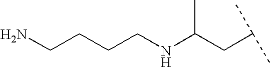 | 577.597 | 577 | 1.808 |
| 6-31 | 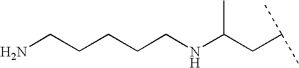 | 591.624 | 591 | 1.808 |
| 6-32 | 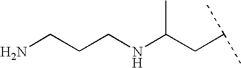 | 563.57 | 563 | 1.802 |
| 6-33 | 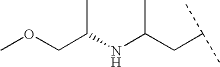 | 578.581 | 578 | 1.915 |
| 6-34 | 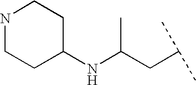 | 589.608 | 589 | 1.796 |
| 6-35 | 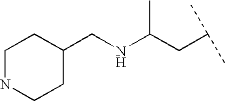 | 603.635 | 603 | 1.808 |
| 6-36 | 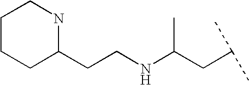 | 617.661 | 617 | 1.823 |
| 6-37 | 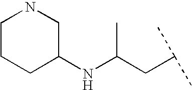 | 589.608 | 589 | 1.802 |
| 6-38 | 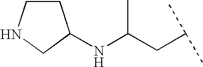 | 575.581 | 575 | 1.802 |
| 6-39 | 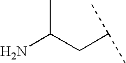 | 506.475 | 506 | 1.879 |
| 6-40 | 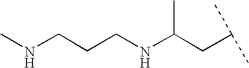 | 577.597 | 577 | 2.118 |
| 6-41 | 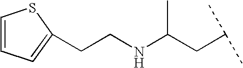 | 616.654 | 616 | 1.982 |
| 6-42 | 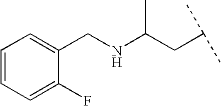 | 628.616 | 628 | 2.011 |
| 6-43 | 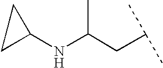 | 546.539 | 546 | 1.885 |
| 6-44 | 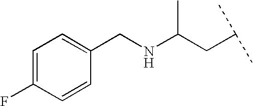 | 614.589 | 614 | 1.99 |
| 6-45 | 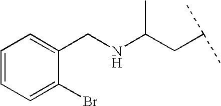 | 675.495 | 674 | 2.01 |
| 6-46 | 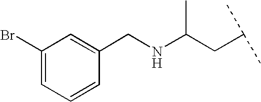 | 675.495 | 674 | 2.021 |
| 6-47 | 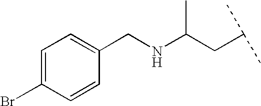 | 675.495 | 674 | 2.026 |
| 6-48 | 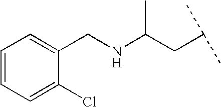 | 631.044 | 630 | 2.002 |
| 6-49 | 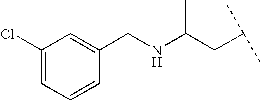 | 631.044 | 630 | 2.011 |
| 6-50 | 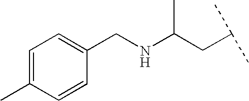 | 631.044 | 630 | 2.018 |
| 6-51 | 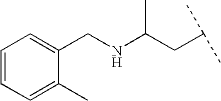 | 610.626 | 610 | 2.001 |
| 6-52 | 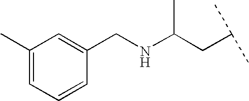 | 610.626 | 610 | 2.01 |
| 6-53 | 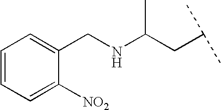 | 643.612 | 641 | 1.98 |
| 6-54 | 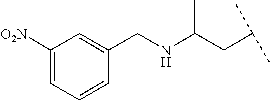 | 643.612 | 641 | 1.986 |
| 6-55 | 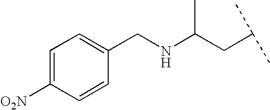 | 643.612 | 641 | 1.983 |
| 6-56 | 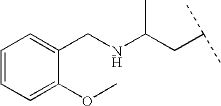 | 626.625 | 626 | 1.987 |
| 6-57 | 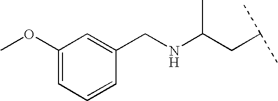 | 626.625 | 626 | 1.984 |
| 6-58 | 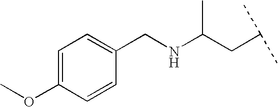 | 626.625 | 626 | 1.986 |
| 6-59 | 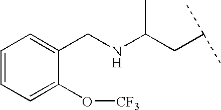 | 680.595 | 680 | 2.06 |
| 6-60 | 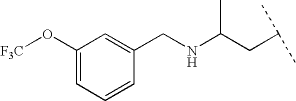 | 680.595 | 680 | 2.067 |
| 6-61 | 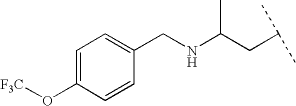 | 680.595 | 680 | 2.068 |
| 6-62 | 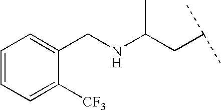 | 664.596 | 664 | 2.033 |
| 6-63 | 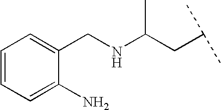 | 611.614 | 611 | 1.953 |
| 6-64 | 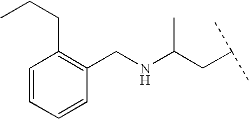 | 640.652 | 640 | 2.026 |
| 6-65 | 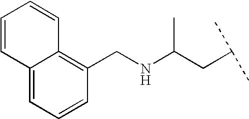 | 646.659 | 646 | 2.034 |
| 6-66 | 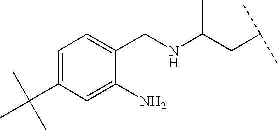 | 652.706 | 652 | 2.115 |
| 6-67 | 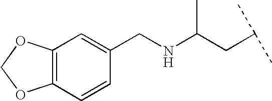 | 640.608 | 640 | 1.976 |
| 6-68 | 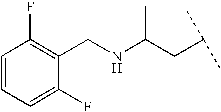 | 632.579 | 632 | 1.977 |
| 6-69 | 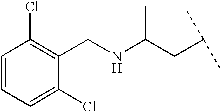 | 665.489 | 664 | 2.015 |
| 6-70 | 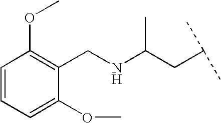 | 656.651 | 656 | 2.013 |
| 6-71 | 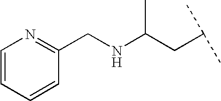 | 597.587 | 597 | 1.942 |
| 6-72 | 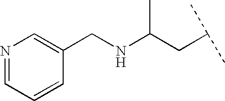 | 597.587 | 597 | 1.856 |
| 6-73 | 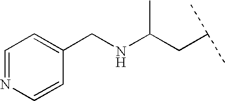 | 597.587 | 597 | 1.84 |
| 6-74 | 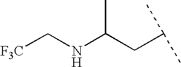 | 588.499 | 588 | 1.933 |
| 6-75 | 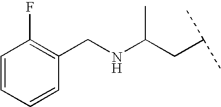 | 614.589 | 614 | 21.792 |
| 6-76 | 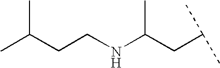 | 576.609 | 576 | 1.446 |
| 6-77 | 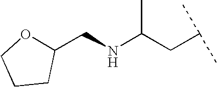 | 590.592 | 590 | 1.532 |
| 6-78 | 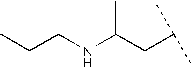 | 548.555 | 562 | 1.501 |
| 6-79 | 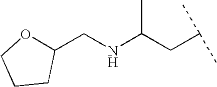 | 590.592 | 590 | 1.504 |
| 6-80 | 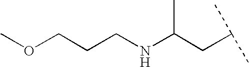 | 578.581 | 578 | 1.509 |
| 6-81 | 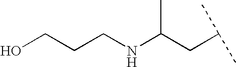 | 564.554 | 564 | 1.518 |
| 6-82 | 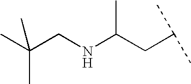 | 576.609 | 576 | 1.475 |
| 6-83 | 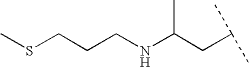 | 594.648 | 594 | 1.461 |
| 6-84 | 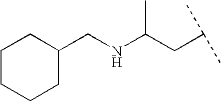 | 602.646 | 602 | 1.504 |
| 6-85 | 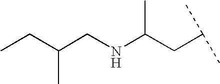 | 576.609 | 576 | 1.493 |
| 6-86 | 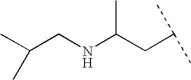 | 564.554 | 564 | 1.471 |
| 6-87 | 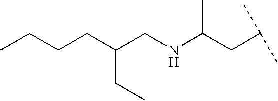 | 618.689 | 618 | 1.542 |
| 6-88 | 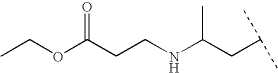 | 606.591 | 606 | 1.448 |
| 6-89 | 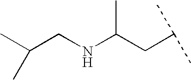 | 562.582 | 562 | 1.649 |
| 6-90 | 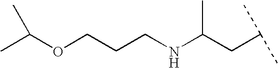 | 606.635 | 606 | 1.633 |
| 6-91 | 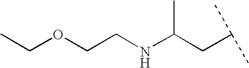 | 578.581 | 578 | 1.607 |
| 6-92 | 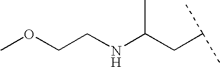 | 564.554 | 564 | 1.584 |
| 6-93 | 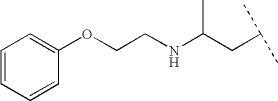 | 626.625 | 626 | 1.591 |
| 6-94 | 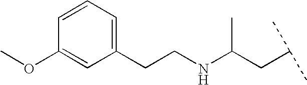 | 640.652 | 640 | 1.643 |
| 6-95 | 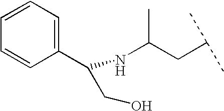 | 626.625 | 626 | 1.551 |
| 6-96 | 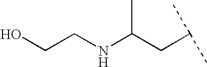 | 550.527 | 550 | 1.623 |
| 6-97 | 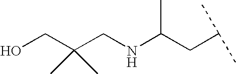 | 592.608 | 592 | 1.739 |
| 6-98 | 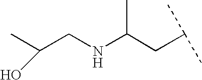 | 564.554 | 564 | j.597 |
| 6-99 | 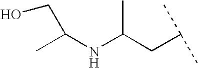 | 564.554 | 564 | 1.708 |
| 6-100 | 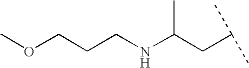 | 578.581 | 578 | 1.667 |
| 6-101 | 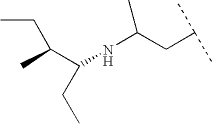 | 606.635 | 606 | 1.68 |
| 6-102 | 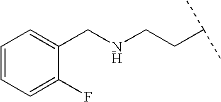 | 600.562 | 600 | 5.094 |
| 6-103 | 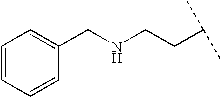 | 582.572 | 582 | 1.437 |
| 6-104 | 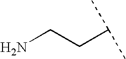 | 492.448 | 492 | 1.535 |
| 6-105 | 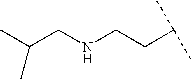 | 548.555 | 548 | 1.51 |
| 6-106 | 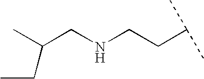 | 562.582 | 562 | 1.499 |
| 6-107 | 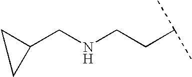 | 546.539 | 546 | 1.59 |
| 6-108 | 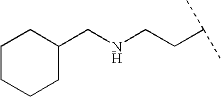 | 588.62 | 588 | 1.576 |
| 6-109 | 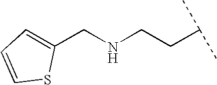 | 588.601 | 588 | 1.44 |
| 6-110 | 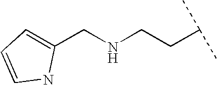 | 571.549 | 571 | 1.517 |
| 6-111 | 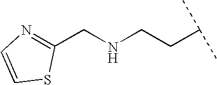 | 589.589 | 589 | 1.297 |
| 6-112 | 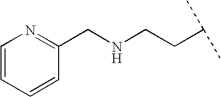 | 583.56 | 583 | 1.504 |
| 6-113 | 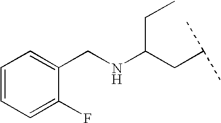 | 628.616 | 628 | 1.35 |
| 6-114 | 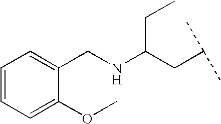 | 640.652 | 640 | 1.646 |
| 6-115 | 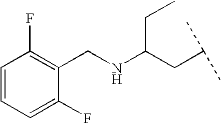 | 646.606 | 646 | 1.285 |
| 6-116 | 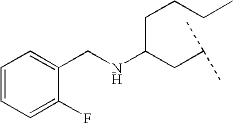 | 656.67 | 656 | 1.359 |
| 6-117 | 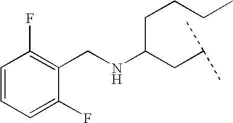 | 674.66 | 674 | 1.26 |
| 6-118 | 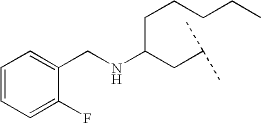 | 670.696 | 670 | 1.354 |
| 6-119 | 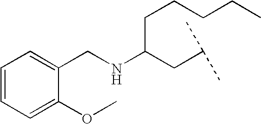 | 682.732 | 682 | 1.603 |
| 6-120 | 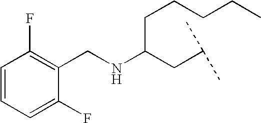 | 688.687 | 688 | 1.255 |
| 6-121 | 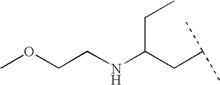 | 578.581 | 578 | 1.526 |
| 6-122 | 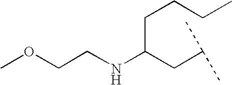 | 606.635 | 606 | 1.465 |
| 6-123 | 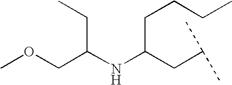 | 634.688 | 634 | 1.514 |
| 6-124 | 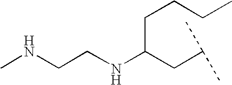 | 605.65 | 605 | 1.597 |
| 6-125 | 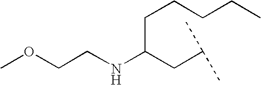 | 620.661 | 620 | 1.503 |
| 6-126 | 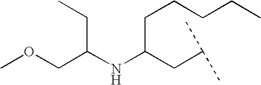 | 648.715 | 648 | 1.627 |
| 6-127 | 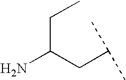 | 520.502 | 520 | 1.22 |
| 6-128 | 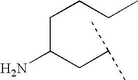 | 548.555 | 548 | 1.169 |
| 6-129 | 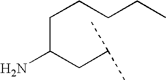 | 562.582 | 562 | 1.159 |

### Example 7

### 1-[2-(2-ETHYLCARBAMATE)-(3R)-(2,4-DICHLOROPHENYL)PROPIONYL]-4-[(2R,S)-(2′-FLUOROBENZYLAMINOPROPYL]PHENYLPIPERAZINE

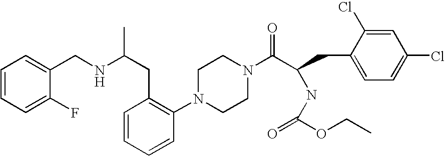

Step 7A: Keto-Phenylpiperazine Derivative 7-1a

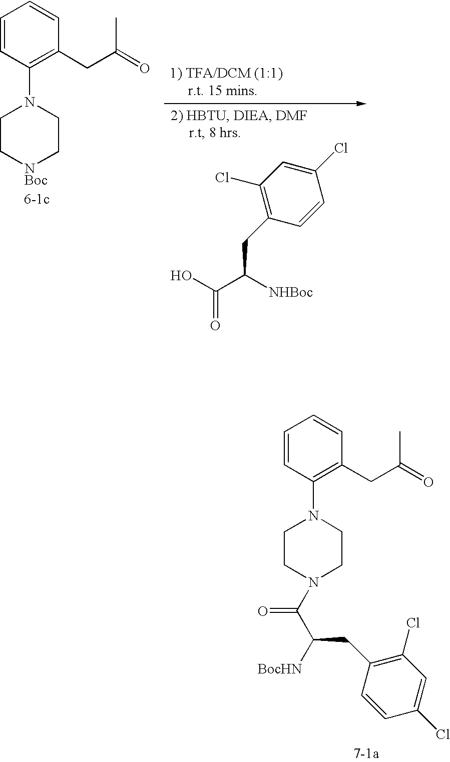

Boc-piperazine phenethyl ketone 6-1c (2.88 g, 9.08 mmol) was dissolved in 16 mL of (1:1) trifluoroacetic acid/dicholormethane and stirred at room temperature for 20 minutes. The reaction mixture was evaporated to dryness, redissolved in dichloromethane (20 mL), and washed with saturated NaHCO₃ solution (3×20 mL). The organic layer was additionally washed with 20 mL of saturated NaCl solution, dried over anhydrous Na₂SO₄, filtered, and solvent removed in vacuo. This deprotected keto-phenylpiperazine intermediate was set aside for later use.

In a separate clean dried flask, Boc-D-2,4-dichlorophenylalanine (2.68 g, 8 mmol) was dissolved in DMF (32 mL) along with diisopropylethyl amine (2.8 mL, 16 mmol) and HBTU (3 g, 8 mmol). The reaction mixture was allowed to stir at room temperature for 1 hour then deprotected keto-phenylpiperazine (prepared above, 1.7 g, 8 mmol) was added along with an additional 2.8 mL of diisopropylethyl amine (16 mmol). The reaction was allowed to stir at room temperature for an additional 8 hours. The reaction mixture was then diluted with ethyl acetate (100 mL) and was washed with water (3×100 mL) and saturated NaCi solution (100 mL). The organic layer was dried over anhydrous MgSO₄, filtered, and solvent removed in vacuo. The product was recovered in 55% yield (2.4 g, 4.4 mmol) after purification by column chromatography on silica using 35% ethyl acetate/hexanes as the eluent (R_{f}=0.3).

Step 7B: 2-Fluorobenzylamino Phenylpiperazine Derivative 7-1b

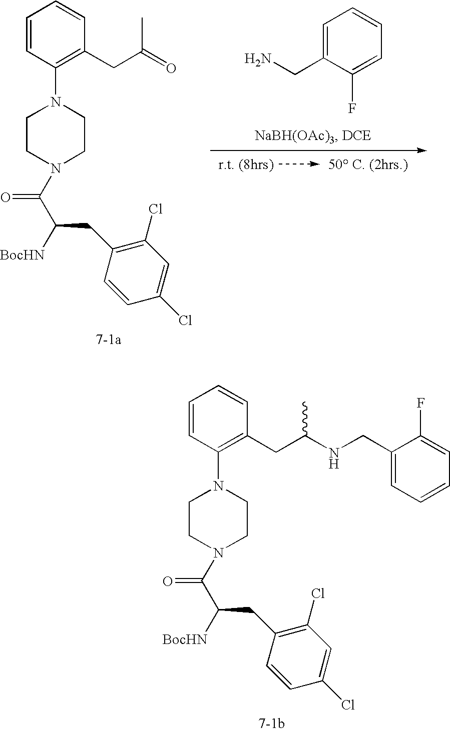

Keto-phenylpiperazine 7-1a (2.36 g, 4.4 mmol) was dissolved in 22 mL of 1,2-dichloroethane. To the reaction flask, 2-fluorobenzyl amine (0.5 mL, 4.4 mmol) and glacial acetic acid (0.25 mL, 4.4 mmol) were added along with NaBH(OAc)₃ (1.3 g, 6.2 mmol). The reaction mixture was allowed to stir for 8 hours at room temperature then was quenched with 20 mL of 1N NaOH solution. The product was extracted with dichloromethane (2×50 mL) then organic layer washed with 50 mL of saturated NaCl solution, dried over anhydrous MgSO₄, filtered, and solvent removed in vacuo. No further purification was needed.

Step 7C: FMOC-2-Fluorobenzylamino Phenylpiperazine derivative 7-1c

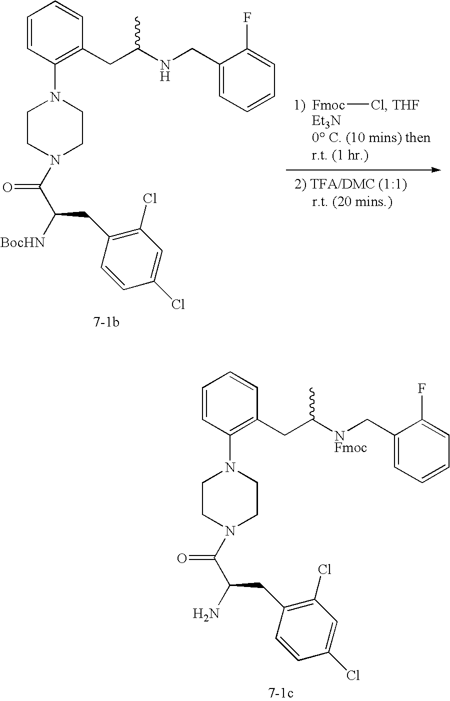

In a clean dried flask, 2-fluorobenzylamino phenylpiperazine 7-1b (2.85 g, 4.44 mmol) was dissolved in 18 mL of THF along with Et₃N (0.67 mL, 4.8 mmol) and cooled to 0° C. To the reaction mixture, 9-fluorenylmethyl chloroformate (1.14 g, 4.4 mmol) was added and the reaction was allowed to stir at 0° C. for 10 minutes followed by stirring at room temperature for 1 hour. The reaction mixture was then evaporated to dryness and the crude product was purified by column chromatography on silica using 27% ethyl acetate/hexanes as the eluent (R_{f}=0.3). The intermediate product, which was recovered in 66% yield (2.54 g), was then dissolved in 20 mL of trifluoroacetic acid/dicholoromethane (1:1) and stirred at room temperature for 20 minutes. The reaction mixture was evaporated to dryness, redissolved in dichloromethane (50 mL), and washed with saturated NaHCO₃ solution (3×50 mL). The organic layer was additionally washed with 50 mL of saturated NaCl solution, dried over anhydrous Na₂SO₄, filtered, and the solvent removed in vacuo. No further purification was needed.

Step 7D: 2-Fluorobenzylamino-phenylpiperazine Carbamate Derivative 7-1

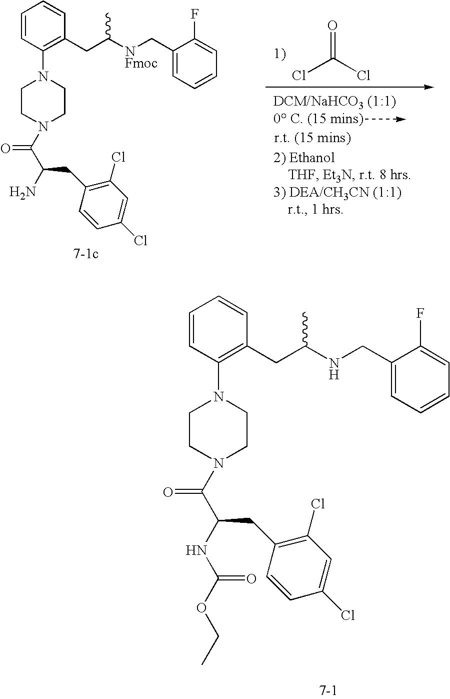

Fmoc-2-fluorobenzylamino phenylpiperazine 7-1c (1.4 g, 1.8 mmol) was dissolved in 10 mL of dichloromethane. To the reaction flask, 10 mL of saturated NaHCO₃ solution was added and the mixture was cooled to 0° C. To the organic layer, phosgene (1.93 M in toluene, 1.24 mL, 2.4 mmol) was added via syringe in one portion and reaction mixture was allowed to stir at 0° C. for 15 minutes followed by 15 minutes at room temperature. The organic layer was separated and washed with saturated NaHCO₃ solution (2×50 mL) followed by washing with 50 mL of saturated NaCl solution. The organic layer was then dried over anhydrous Na₂SO₄, filtered, and solvent removed in vacuo. The residue was dissolved in 12 mL of THF to make a 0.15 M 2-fluorobenzylamino phenylpiperazine isocyanate stock solution.

In a 4 mL reaction vial, a 1 mL aliquot of the 0.15 M 2-fluorobenzylamino phenylpiperazine isocyanate stock solution was added along with Et₃N (20.38 uL, 0.15 mmol). To the reaction vial, ethanol (10.2 uL, 0.3 mmol) were added and the reaction was allowed to stir at room temperature for 8 hours. The solvent was then removed by evaporation under a stream on nitrogen and the residue was dissolved in 4 mL of diethylamine/acetonitrile solution (1:1). The reaction mixture was allowed to stir at room temperature for 1 hour then was evaporated to dryness. The residue was dissolved in 1 mL of methanol and the crude product was purified by preparative HPLC. Compound 7-1 was recovered as the TFA salt in 33% yield. MS: calc. for C₃₂H₃₇C₁₂FN₄O: 614.2; Found: 615 (M+H); retention time: 6.74 minutes; Method info: APCI positive ion scan 100-1000 Frag V=80; 95% 0.05% TFA/H₂O to 95% ACN/0.05% TFA over 13 min, 15.5 min run, ODS-AQ column

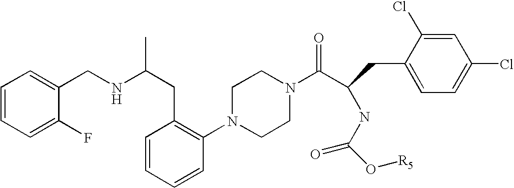

| | | Formula | | Retention |
|---|---|---|---|---|
| Cpd | R₅— | Weight | Mass | Time |
| 7-1 | ethyl | 615.573 | 615 | 6.744 |
| 7-2 | benzyl | 677.644 | 677 | 7.537 |
| 7-3 | isobutyl | 643 .627 | 643 | 7.429 |
| 7-4 | 2-F-ethyl | 633.563 | 633 | 6.611 |
| 7-5 | n-propyl | 629.6 | 629 | 7.158 |
| 7-6 | isopropyl | 629.6 | 629 | 7.166 |
| 7-7 | n-butyl | 643.627 | 643 | 7.541 |
| 7-8 | sec-butyl | 643 .627 | 643 | 6.9 16 |
| 7-9 | cyclopentyl | 655.638 | 655 | 7.552 |
| 7-10 | cyclohexyl | 669.665 | 669 | 7.931 |
| 7-11 | cyclopropyl-CH₂— | 641.611 | 641 | 7.211 |
| 7-12 | cyclobutyl-CH₂— | 655.638 | 655 | 7.653 |
| 7-13 | cyclopentyl-CH₂— | 669.665 | 669 | 7.987 |
| 7-14 | cyclohexyl-CH₂— | 683.692 | 683 | 8.306 |

### Example 8

### 1-[2-(2-ISOPROPYLUREA)-(3R)-(2,4-DICHLOROPHENYL)PROPIONYL]-4-[(2R,S)-(2′-FLUOROBENZYLAMINOPROPYL]PHENYLPIPERAZINE

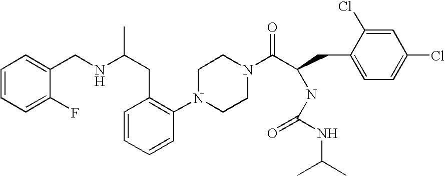

Step8A: 2-Fluorobenzylamino-phenylpiperazine Carbamate Derivative 8-1

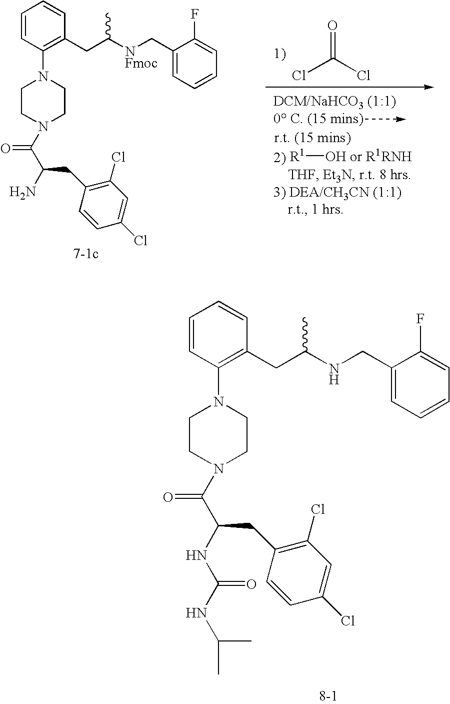

Fmoc-2-fluorobenzylamino phenylpiperazine 7-1c (1.4 g, 1.8 mmol) was dissolved in 10 mL of dichloromethane. To the reaction flask, 10 mL of saturated NaHCO₃ solution was added and the mixture was cooled to 0° C. To the organic layer, phosgene (1.93 M in toluene, 1.24 mL, 2.4 mmol) was added via syringe in one portion and reaction mixture was allowed to stir at 0° C. for 15 minutes followed by 15 minutes at room temperature. The organic layer was separated and washed with saturated NaHCO₃ solution (2×50 mL) followed by washing with 50 mL of saturated NaCl solution. The organic layer was then dried over anhydrous Na₂SO₄, filtered, and solvent removed in vacuo. The residue was dissolved in 12 mL of THF to make a 0.15 M 2-fluorobenzylamino phenylpiperazine isocyanate stock solution.

In a 4 mL reaction vial, a 1 mL aliquot of the 0.15M 2-fluorobenzylamino phenylpiperazine isocyanate stock solution (prepared above) was added along with Et₃N (20.38 uL, 0.15 mmol). To the reaction vial, isopropylamine (12.8 uL, 0.15 mmol) was added and the reaction was allowed to stir at room temperature for 8 hours. The solvent was then removed by evaporation under a stream on nitrogen and the residue was dissolved in 4 mL of diethylamine/acetonitrile solution (1:1). The reaction mixture was allowed to stir at room temperature for 1 hour then evaporated to dryness. The residue was dissolved in 1 mL of methanol and the crude product was purified by preparative HPLC. The compound was recovered as the TFA salt in 33% overall yield from compound 8-1. MS: calc. for C₃₃H₄₀Cl₂FN₅O₂: 628.6; Found: 628.1 (M); retention time: 6.45 minutes; Method info: APCI positive ion scan 100-1000 Frag V=80; 95% 0.05% TFA/H₂O to 95% ACN/0.05% TFA over 13 min, 15.5 min run, ODS-AQ column

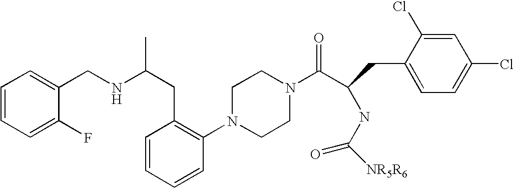

| | | Formula | | Retention |
|---|---|---|---|---|
| Cpd | R₅R₆N— | Weight | Mass | Time |
| 8-1 | (isopropyl)NH— | 628.616 | 628 | 6.454 |
| 8-2 | (cyclopentyl)NH— | 654.654 | 654 | 6.843 |
| 8-3 | (ethyl)₂N— | 642.643 | 642 | 6.849 |

### Example 9

### 1-[2-(3-METHYLBUTYROYL)PHENYL]-4-[(2R)-(3-AMINOPROPIONYLAMIDO)-3-(2,4-DICHLOROPHENYL)PROPIONYL]PIPERAZINE

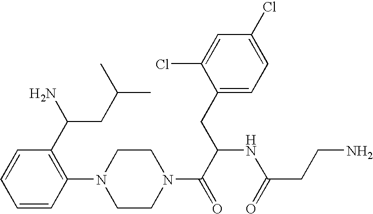

Step 9A: 2-(2-Methylpropyl) fluorophenyl ketone 9-1a

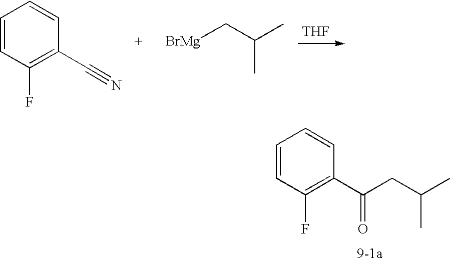

To 12.11 g (100 mmol) of 2-fluorobenzonitrile in 40 mL of THF, 2.0 M isobutyl magnesium bromide (60 mL, 120 mmol) was added dropwise and stirred at RT for 2 hours. The mixture was quenched with saturated aqueous ammonium chloride and then was extracted with ethyl acetate. After removal of solvents gave 13.3 g of 2-(2-methylpropyl) fluorophenyl ketone, compound 9-1a (GC 99+%; M⁺180). Yield 74%.

Step 9B: 1-[2-(3-Methylbutyroyl)phenyl]-4-(tert-butoxycarbonyl)piperazine 9-1b

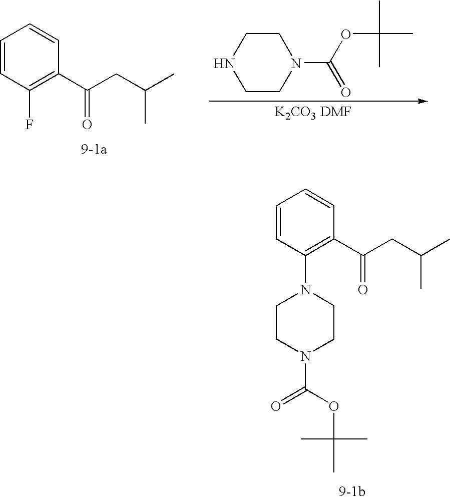

2-(2-Methylpropyl) fluorophenyl ketone 9-1a (10.81 g, 60 mmol), 11.18 g (60 mmol) of BOC-piperazine, 16.59 g (120 mmol) of potassium carbonate and 60 mL of DMF were heated to 130° C. for 10 hours, with stirring. The mixture was cooled, dissolved in water and extracted with ethyl acetate. Purification on silica gel (hexanes/EtOAc 9:1 as elutant) gave 12.9 g of compound 9-1b (62% yield). M⁺288.1.

Step 9C: N—BOC-β-Ala-D-2,4-di-Cl-PheOH dipeptide 9-1c

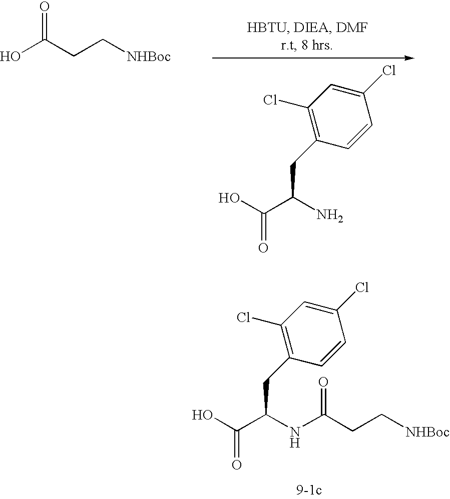

In a clean dried flask, Boc-B-alanine dipeptide (72.7 g, 384.5 mmol) was dissolved in DMF (1.64 L) along with diisopropylethyl amine (201 ML, 18.8 mmol) and HBTU (145.8 g, 384.5 mmol). The reaction mixture was allowed to stir at room temperature for 1 hour then 2,4-dichlorophenylalanine (90 g, 384.5 mmol) was added to the reaction mixture. The reaction was allowed to stir at room temperature for an additional 8 hours. The reaction mixture was diluted with ethyl acetate (2.5 L), and was washed with 1N citric acid (3×1.5 L) and saturated NaCl solution (2L). The organic layer was dried over anhydrous MgSO₄, filtered, and solvent removed in vacuo. The product was recovered as a slightly tan yellow solid in 68% yield (106.4 g) without further purification.

Step 9D: 1-[2-(3-Methylbutyroyl)phenyl]-4-{(2R)-[3-(tert-butoxycarbonylamino)propionylamido]}-3-(2,4-dichlorophenyl)propionyl]piperazine 9-1d

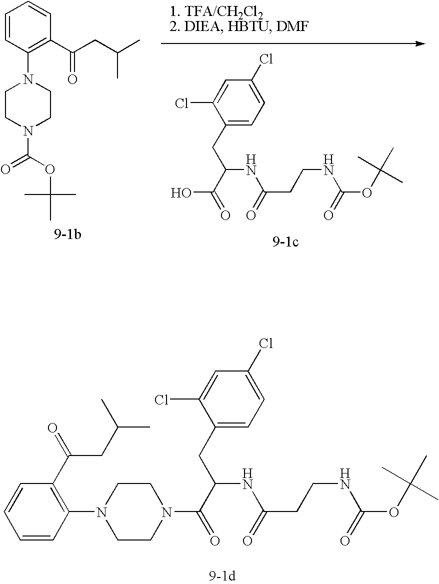

1.72 g (6 mmol) of 1-[2-(3-methylbutyroyl)phenyl]-4-tert-butoxycarbonylpiperazine 9-1b and 18 mL of TFA/CH₂Cl₂ mixture (1:1) were stirred vigorously for 30 minutes at the RT. The solvents removed in vacuo, 18 mL of methylene chloride and 10 mL of diisopropyl ethylamine were added and stirred for 5 minutes. The solvents were removed and the residue was dissolved in 5 mL of DMF and added to a mixture of N—BOC-β-Ala-D-2,4-di-Cl-PheOH dipeptide 9-1c (2.00 g, 6 mmol) and 2.74 g (7.2 mmol) of HBTU in 10 mL of DMF and stirred at 40° C. for 10 hours. The reaction mixture was treated with water, extracted with ethyl acetate and purified on silica (hexane/ethyl acetate 1:1) to give 2.20 g of 9-1d. Yield=58%. M+1⁺634.2.

Step 9E: 1-{2-[(1R,S)-amino-3-methylbutyroyl]phenyl}-4-[(2R)-(3-aminopropionylamido)-3-(2,4-dichlorophenyl)propionyl]piperazine 9-1

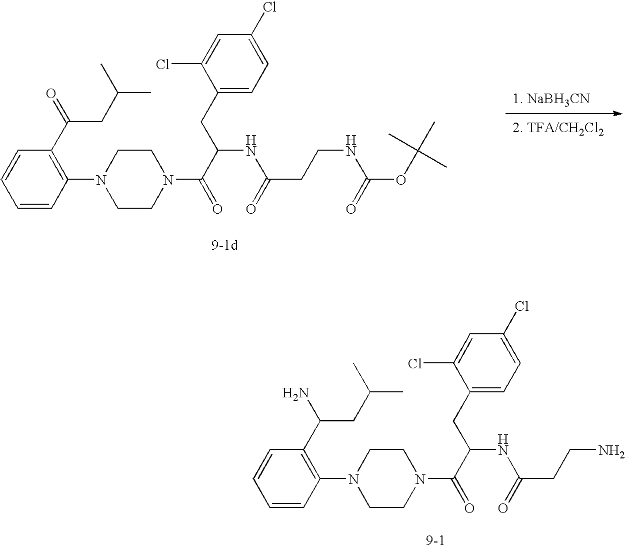

1-[2-(3-Methylbutyroyl)phenyl]-4-{(2R)- [3-(tert-butoxycarbonylamino)pro-pionylamido]}-3-(2,4-dichlorophenyl)propionyl]piperazine 9-1d (317 mg, 0.5 mmol),) ammonium acetate (1.16 g, 15 mmol and 5 mL of 2-propanol were stirred at 70° C. for 2 hours. 220 mg (3.5 mmol) of sodium cyanoborohydride was added in 4 portions over 2 hours and the mixture stirred for another 2 hours at 70° C. Solvents were evaporated and the residue was dissolved in water and extracted with ethyl acetate. Purified on silica (hexane/ethyl acetate 1:1). After removal of solvents the BOC intermediate was treated with 500 μL of TFA/CH₂Cl₂ mixture (1:1) and stirred vigorously for 30 minutes at room temperature. Following removal of the solvents, title compound 9-1 was obtained as a TFA salt. M+1⁺534.1.

### Example 10

### 1-{2-[2-(2-THIOPHENYL)ETHYLAMINOMETHYL]PHENYL-4-[2-AMINOMETHYL-3-(4-CHLOROPHENYL)PROPIONYL]PIPERAZINE

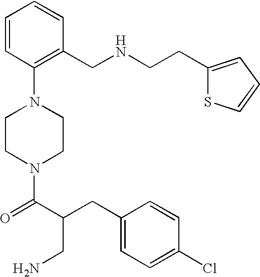

Step 10A:

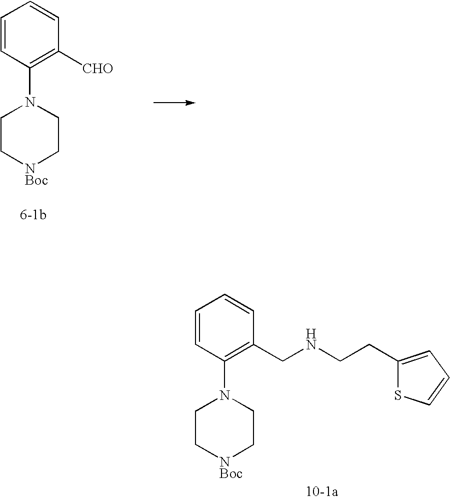

To a solution of the aldehyde_{—}6-1b (2.70 g, 5.35 mmol) and 2-thiopheneethylamine (0.713 g, 5.62 mmol) in dichloromethane (30 mL) was added sodium triacetoxyborohydride (1.59 g, 7.50 mmol). The mixture was stirred overnight, then washed with saturated aqueous sodium bicarbonate solution (15 mL), dried over magnesium sulfate, and evaporated at reduced pressure to give the amine 10-1a as a yellow foam (3.05 g; MS=617.2 (M+H)⁺).

Step 10B:

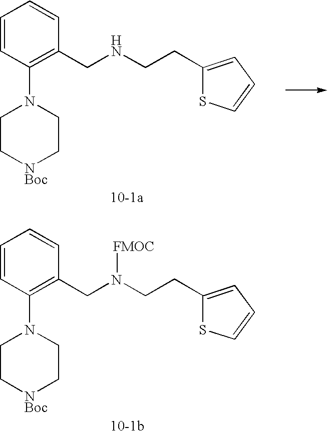

A portion of the amine 10-1a (1.22 g, 1.98 mmol) was immediately dissolved in dichloromethane (5 mL) and cooled in an ice-bath. FMOC—Cl (0.51 g, 1.98 mmol) and triethylamine (0.3 mL) were added, and the mixture was stirred for 0.5 h. The mixture was loaded directly onto a silica gel column and was eluted (40% ethyl acetate/hexane) to provide the FMOC-protected amine 10-1b as a yellow foam (1.61 g).

Step 10C:

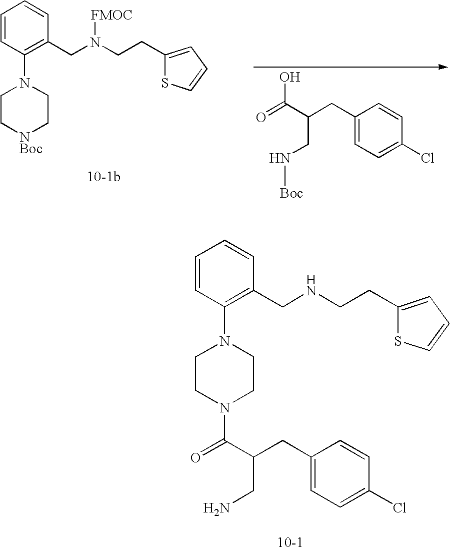

To a dichloromethane (0.8 mL) solution of 10-1b (50.0 mg) was added trifluoroacetic acid (0.2 mL) at 23° C. and the mixture was stirred for 50 minutes. The reaction mixture was neutralized with saturated aqueous NaHCO₃ solution (5 mL) and extracted with EtOAc (30 mL). The organic layer was dried over Na₂SO₄ and evaporated to provide the piperazine as white foam, which was dissolved in DMF/dichloromethane (1:3, 1 mL). To this solution was added NaHCO₃ (16.2 mg, 0.192 mmol), 2-(Boc-aminomethyl)-3-(4-dichloro-phenyl)propionic acid (30 mg, 0.12), (HOBt (15.5 mg, 0.12 mmol), EDCI (22.0 g, 0.12 mmol) sequentially. The reaction mixture was stirred overnight at room temperature. The mixture was diluted with EtOAc (20 mL), washed with 5% aqueous HCl (5 mL), saturated aqueous NaHCO₃ (5 mL), brine (5 mL), and was dried (Na₂SO₄). The solution was concentrated in vacuo to provide protected product, which was dissolved in dichloromethane (2 mL) and treated with TFA. The mixture was stirred for 1 h at room temperature. The excess of TFA and solvent were removed in vacuo. The residue was purified by flash column chromatography (5˜15% MeOH in dichloromethane) to provide product 10-1 as a colorless oil.

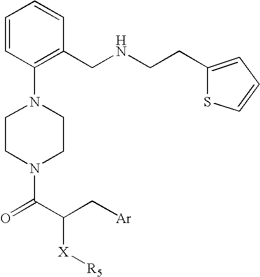

| Cpd | Ar | —X—R₅ | Mass | Mol Wt |
|---|---|---|---|---|
| 10-1 | 4-Cl-phenyl | —H | 468 | 468.06 |
| 10-2 | 4-Cl-phenyl | —CH₂NH₂ | 497 | 497.10 |
| 10-3 | 4-Cl-phenyl | —OH | 484 | 484.06 |
| 10-4 | 4-Cl-phenyl | —NH₂ | 483 | 483.08 |
| 10-5 | 2-Cl-phenyl | —NH₂ | 483 | 483.08 |
| 10-6 | 2,4-Cl-phenyl | —NH₂ | 517 | 517.52 |

### Example 11

### (1S)-3-METHYL-1-(2-{4-[3-(2,4-DICHLORO-PHENYL)PROPIONYL]-PIPERAZINYL}-5-TRIFLUOROMETHYL-PHENYL)BUTYLAMINE

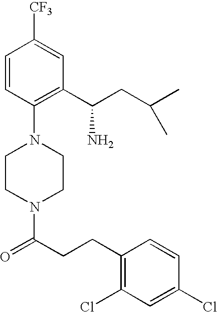

Step 11A: 2-[4′-(tert-Butoxycarbonyl)-piperazinyl]-5-trifluoromethyl-benzaldehyde

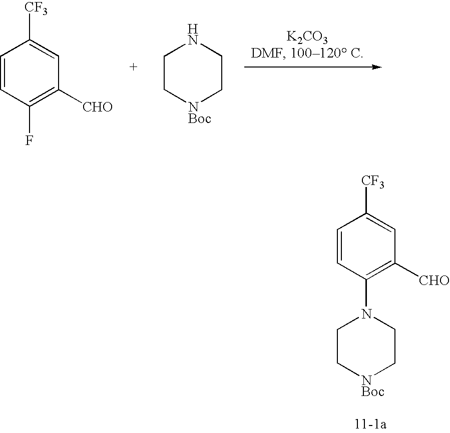

To a solution of 2-fluoro-5-trifluoromethyl-benzaldehyde (10.0 mL, 68.7 mmol) and 1-BOC-piperazine (15.4 g, 82.4 mmol) in 140 mL of DMF was added K₂CO₃ (47.4 g, 344 mmol). The reaction mixture was heated and stirred at 120° C. The reaction was monitored by TLC and LC/MS. After 10 hours of stirring, the reaction mixture was cooled to room temperature and diluted with 200 mL of EtOAc. The mixture was filtered, and the filter was washed well with EtOAc (3×50 mL). The filtrate was washed with 5% aqueous HCl (100 mL) and the aqueous layer was extracted with EtOAc (3×25 mL). The combined organic layers were washed with H₂O (2×40 mL), brine (50 mL), dried (MgSO₄), and concentrated in vacuo. The residue was triturated with hexanes (3×20 mL) to form a brown oil. The brown oil slowly solidified to give the 11-1a as a yellow solid. (22.3 g, 92%).

Step 11B: (S)—N-{2-[4′-(tert-Butoxycarbonyl)-piperazinyl]-5-trifluoromethyl-benzylidene}-t-butanesulfinamide

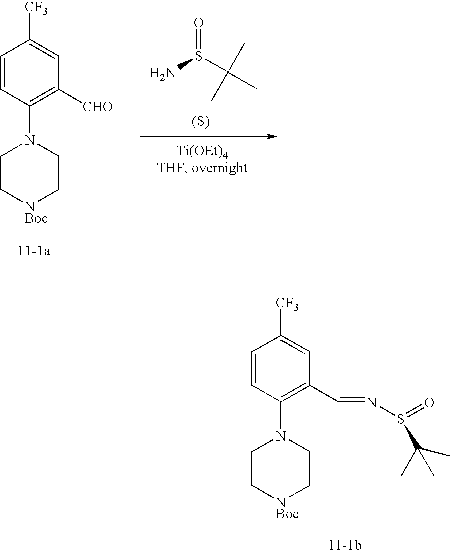

To a THF (41 mL) solution of aldehyde 11-1a (3.29 g, 9.18 mmol) at room temperature was added Ti(OEt)₄ (tech. Grade, Ti˜20%, contains excess ethanol, 9 mL, 36.7 mmol) and (S)-(−)-2-methyl-2-propanesulfinamide (1.26 g, 10.1 mmol). The mixture was stirred overnight. The reaction mixture was poured to a saturated aqueous NaCl solution (30 mL) at room temperature with vigorous stirring and the resulting suspension was filtered through Celite and the filter cake was washed with EtOAc (500 mL). The aqueous layer was extracted with EtOAc (30 mL) and the combined organic layers were dried over Na₂SO₄ and evaporated to provide a residue which was purified by 5˜10% EtOAc/Hexanes triturating to give 4.20 g of compound 11-1b as a light yellow powder (99%).

Step 11C: (S)—N-{2-[4′-(tert-Butoxycarbonyl)-piperazinyl]-5-trifluoromethyl-benzylidene}-iso-butyl-t-butanesulfinamide

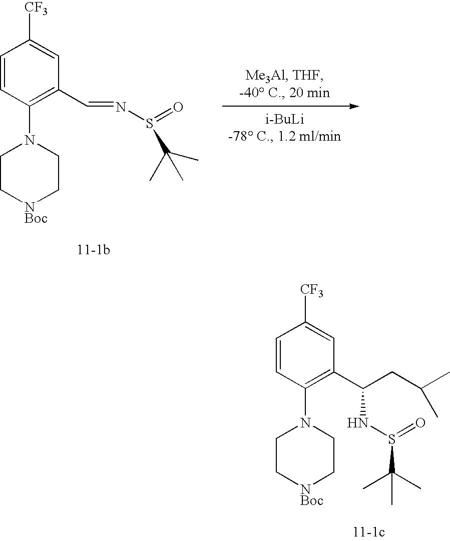

To a THF (25 mL) solution of sulfinyl aldimine 11-1b (4.20 g, 9.10 mmol) was added trimethylaluminum (2.0 M in toluene or heptane or hexane, 9.10 mL, 18.2 mmol) at −40° C. and the mixture was stirred for 20 minutes. The mixture was cooled to −78° C. and i-BuLi (1.6 M in heptane from Fluka, 11.4 mL, 18.2 mmol) was added by syringe pump at 1.2 mL/min. The reaction mixture was stirred for 30 minutes at −78° C., quenched with a 5% aqueous HCl (25 mL) at −78° C., warmed to 10° C. and extracted with EtOAc (3×50 mL). The combined organic layers were washed with brine (30 mL), dried over Na₂SO₄ and evaporated to provide a crude oil which was purified by 10˜25% EtOAc/Hexanes chromatography to give 4.00 g of compound 11-1c as a white foam (85% yield).

Step 11D: (1S)-3-Methyl-1-(2-{4-[3-(2,4-dichloro-phenyl)propionyl]-piperazinyl}-5-trifluoromethyl-phenyl)butylamine

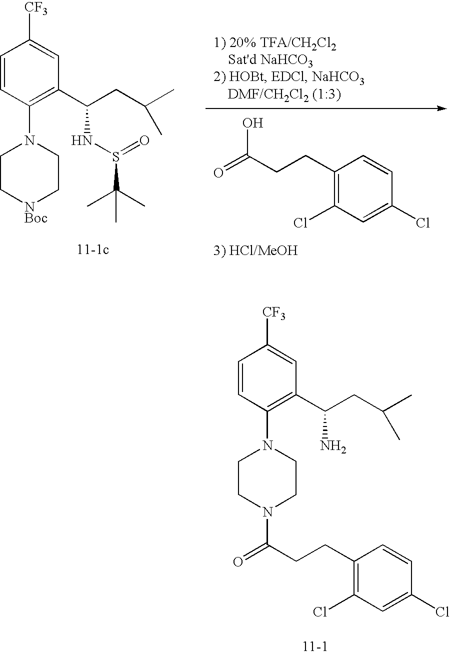

To a dichloromethane (0.8 mL) solution of BOC-piperazine 11-1c (50.0 mg, 0.096 mmol) was added trifluoroacetic acid (0.2 mL) at 23° C. and the mixture was stirred for 50 minutes. The reaction mixture was treated with saturated aqueous NaHCO₃ solution (5 mL) and extracted with EtOAc (30 mL). The organic layer was dried over Na₂SO₄ and evaporated to provide the piperazine as white foam, which was dissolved in DMF/methylene chloride (1:3, 1 mL). To this solution was added NaHCO₃ (16.2 mg, 0.192 mmol), 3-(2,4-dichloro-phenyl)propionic acid (25.3 g, 0.12 mmol), (HOBt (15.5 mg, 0.12 mmol), and EDCI (22.0 g, 0.12 mmol) sequentially. The reaction mixture was stirred overnight at room temperature. The mixture was diluted with EtOAc (20 mL), washed with 5% aqueous HCl (5 mL), saturated aqueous NaHCO₃ (5 mL), brine (5 mL), and then was dried (Na₂SO₄). The solution was concentrated in vacuo to provide crude product, which was dissolved in MeOH (2 mL) and treated with HCl (58 mL 4 N HCl in dioxane). The mixture was stirred for 1 h at room temperature. The excess of HCl and solvent were removed in vacuo. The residue was purified by flash column chromatography (5˜15% MeOH in dichloromethane) to provide 11-1 as a colorless oil ( 55.2 mg, 93%).

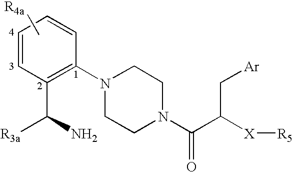

| Cpd | R₃ₐ | R₄ₐ | Ar | X—R₅ | Mass | Mol Wt |
|---|---|---|---|---|---|---|
| 11-1 | (S)-isobutyl | 4-CF₃ | 2,4-Cl-phenyl | H | 516 | 516.43 |
| 11-2 | (S)-isobutyl | 4-CF₃ | 2,4-Cl-phenyl | (R)-NH₂ | 532 | 531.45 |
| 11-3 | (S)-isobutyl | 4-CF₃ | 2,4-Cl-phenyl | (R)-NMe₂ | 560 | 559.51 |
| 11-4 | (S)-isobutyl | 4-CF₃ | 2,4-Cl-phenyl | Me | 530 | 530.47 |
| 11-5 | (S)-isobutyl | 4-CF₃ | 2,6-Cl-phenyl | H | 516 | 516.43 |
| 11-6 | (S)-isobutyl | 4-CF₃ | 2-Cl-phenyl | H | 482 | 481.99 |
| 11-7 | (S)-isobutyl | 4-CF₃ | 2-F-phenyl | H | 465 | 465.53 |
| 11-8 | (S)-isobutyl | 4-CF₃ | 2-OH-phenyl | H | 464 | 463.54 |
| 11-9 | (S)-isobutyl | 4-CF₃ | 2-MeO-phenyl | H | 478 | 477.57 |
| 11-10 | (S)-isobutyl | 4-CF₃ | 3-MeO-phenyl | H | 478 | 477.57 |
| 11-11 | (S)-isobutyl | 4-CF₃ | 3-Me-phenyl | H | 462 | 461.57 |
| 11-12 | (S)-isobutyl | 4-CF₃ | 3-CF₃-phenyl | H | 516 | 515.54 |
| 11-13 | (S)-isobutyl | 4-CF₃ | 4-MeO-phenyl | H | 478 | 477.57 |
| 11-14 | (S)-isobutyl | 4-CF₃ | 4-OH-phenyl | H | 464 | 463.54 |
| 11-15 | (S)-isobutyl | 4-CF₃ | 4-MeSO₂-phenyl | H 536 | 525.63 | |
| 11-16 | (S)-isobutyl | 4-CF₃ | 3,4-methylenedioxy- | H | 492 | 4~,1.55 |
| | | | phenyl | | | |
| 11-17 | (S)-isobutyl | 4-CF₃ | 3,4-MeO-phenyl | H | 508 | 507.59 |
| 11-18 | (S)-isobutyl | 4-CF₃ | 2,5-MeO-phenyl | H | 508 | 507.59 |
| 11-19 | (S)-isobutyl | 4-CF₃ | 2,4,5-MeO-phenyl | H | 538 | 537.62 |
| 11-20 | (S)-sec-butyl | 4-CF₃ | 2,4-Cl-phenyl | H | 517 | 516.43 |
| 11-21 | (S)-isobutyl | 4-F | 2,4-Cl-phenyl | H | 467 | 466.43 |

### Example 12

### N-(2-TETRAHYDROFURAN)METHYL (1S)-2-METHYL-1-(2-{4-[3-(2,4-DICHLORO-PHENYL)PROPIONYL]-PIPERAZINYL}-5-TRIFLUOROMETHYL-PHENYL)BUTYLAMINE

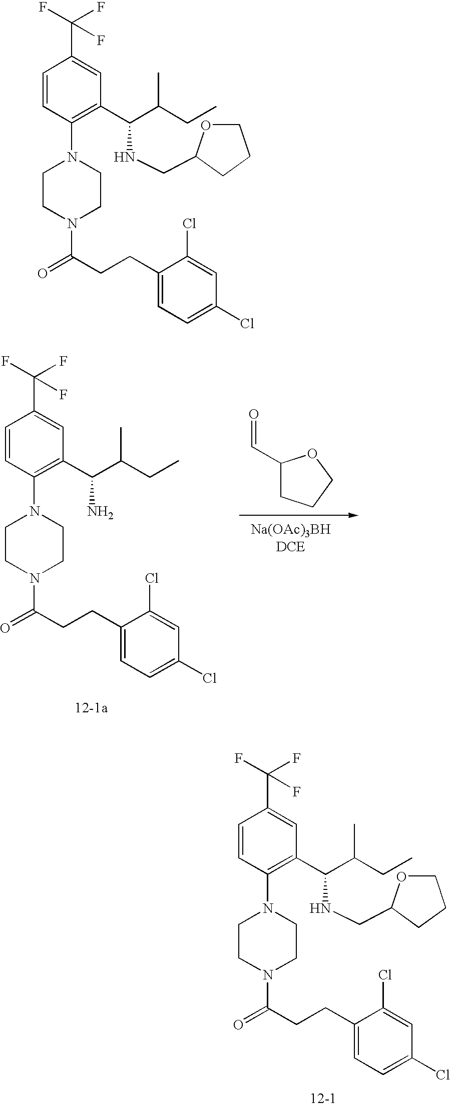

In a 1 dram vial, compound 12-1a (52 mg, 0.1 mmol, made according to the procedure of Example 11) is dissolved in dichloroethane (1 mL) and then treated with tetrahydrofuran-3-carboxaldehyde (20 mg, 0.2 mmol). The vial was capped and the mixture was allowed to stir for 45 minutes at room temperature. Sodium triacetoxy borohydride (42 mg, 0.2 mmol) was added and the mixture stirred for 45 minutes. The mixture was then diluted with dichloromethane (1 mL) and washed once with aqueous NaHCO₃ (1 mL). The organic layer was collected, dried over anhydrous NaSO₄, and filtered. Solvent was reduced under a stream of nitrogen to afford an orange residue. Methanol (2 mL) was added and 1 mL of the solution was purified via prep HPLC to give 2 mg of compound 12-1. LCMS (tᵣ, 7.062) 601 (M+H).

The following compounds were made by the procedures outlined in the Examples.

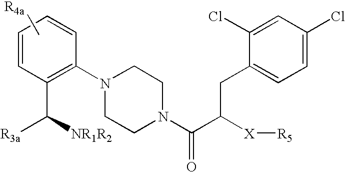

| Cpd | R₃ₐ | R₁R₂N— | R₄ₐ | R₅X— | Mol Wt |
|---|---|---|---|---|---|
| 12-1 | (S)-sec-butyl | 2- | 4-CF₃ | H | 600.549 |
| | | tetrahydrofuranCH₂NH— | | | |
| 12-2 | (S)-isobutyl | MeNH— | 4-CF₃ | Me₂N— | 573.53 |
| 12-3 | (S)-isobutyl | MeNH— | 4-CF₃ | H₂N— | 545.48 |
| 12-4 | (S)-isobutyl | MeNH— | 4-CF₃ | Me | 544.49 |
| 12-5 | (S)-isobutyl | EtNH— | 6-F | H | 494.48 |
| 12-6 | (S)-isobutyl | MeOCH₂CH₂NH— | 6-F | H | 524.50 |
| 12-7 | (S)-isobutyl | MeNH— | 4-F | H | 480.45 |
| 12-8 | (S)-isobutyl | EtNH— | 4-F | H | 494.48 |
| 12-9 | (S)-isobutyl | MeOCH₂CH₂NH— | 4-F | H | 524.50 |
| 12-10 | (S)-isobutyl | MeNH— | 4-CF₃ | H | 530.46 |
| 12-11 | (S)-isobutyl | EtNH— | 4-CF₃ | H | 544.49 |
| 12-12 | (S)-isobutyl | MeOCH₂CH₂NH— | 4-CF₃ | H | 574.51 |
| 12-13 | (S)-sec-butyl | MeNH— | 4-CF₃ | H | 530.459 |
| 12-14 | (S)-sec-butyl | EtNH— | 4-CF₃ | H | 544.486 |
| 12-15 | (S)-sec-butyl | PhCH₂CH₂NH— | 4-CF₃ | H | 620.583 |
| 12-16 | (S)-sec-butyl | 2-F-Bn-NH— | 4-CF₃ | H | 624.547 |
| 12-17 | (S)-sec-butyl | Bn-NH— | 4-CF₃ | H | 606.556 |
| 12-18 | (S)-sec-butyl | NH₂CH₂CH₂NH— | 4-CF₃ | H | 559.5 |
| 12-19 | (S)-sec-butyl | EtCH(Me)CH₂NH— | 4-CF₃ | H | 586.566 |
| 12-20 | (S)-sec-butyl | 4-Py-CH₂NH— | 4-CF₃ | H | 607.544 |
| 12-21 | (S)-sec-butyl | (R)-2-NH₂PrNH— | 4-CF₃ | H | 573.527 |
| 12-22 | isobutyl | H₂N— | H | (R)-n-Pr2N— | 547.61 |
| 12-23 | isobutyl | H₂N— | H | (R)-n-Bu₂N— | 575.66 |
| 12-24 | isobutyl | H₂N— | H | (R)-i-Bu₂N— | 575.66 |
| 12-25 | isobutyl | H₂N— | H | (R)-(c-PrCH₂)₂N— | 571.63 |
| 12-26 | isobutyl | H₂N— | H | (R)-(2-PyCH₂)₂N— | 645.68 |
| 12-27 | (R)-Me | MeOCH₂CH₂NH— | 4-F | MeONHCONH— | 570.50 |

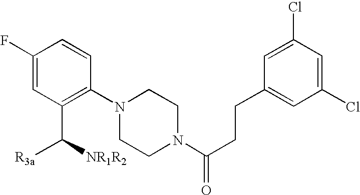

| Cpd | R₃ₐ | R₁R₂N— | Mol Wt |
|---|---|---|---|
| 12-28 | methyl | 2-MeOPhCH₂CH₂NH— | 558.52 |
| 12-29 | methyl | 2-FPhCH₂CH₂NH— | 546.49 |
| 12-30 | methyl | i-PrOCH₂CH₂NH— | 510.48 |
| 12-31 | methyl | EtOCH₂CH₂NH— | 496.45 |
| 12-32 | methyl | MeOCH₂CH₂NH— | 482.424 |
| 12-33 | methyl | MeOCH₂CH(Me)NH— | 496.45 |
| 12-34 | methyl | i-Bu-NH— | 480.45 |
| 12-35 | methyl | Bu-NH— | 480.452 |
| 12-36 | methyl | c-Pr-NH— | 464.41 |
| 12-37 | methyl | MeNH— | 438.37 |
| 12-38 | methyl | 1-pyrrolidine | 478.44 |
| 12-39 | (R)-Me | 1-morpholine | 494.44 |
| 12-40 | (R)-Me | (MeOCH₂CH₂)₂N— | 540.50 |
| 12-41 | Methyl | 2-MeOPhCH₂CH₂NH— | 558.52 |

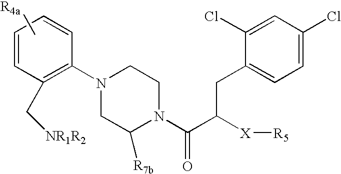

| Cpd | R₄ₐ | R₁R₂N— | R_{7b} | R₅X— | Mol Wt | MS |
|---|---|---|---|---|---|---|
| 12-42 | 4-Br | MeOCH₂CH₂N(Me)— | H | H | 543.33 | 544 |
| 12-43 | 4-Cl | MeOCH₂CH₂N(Me)— | H | H | 498.88 | 499 |
| 12-44 | 4-Br | NH₂CH₂CH₂NH— | H | H | 514.29 | 515 |
| 12-45 | 4-Cl | NH₂CH₂CH₂NH— | H | H | 469.84 | 470 |
| 12-46 | 4-BrMe | OCH₂CH(Me)NH— | H | H | 543.33 | 544 |
| 12-47 | 4-Cl | MeOCH₂CH(Me)NH | H | H | 498.88 | 499 |
| 12-48 | 4-Cl | MeOCH₂CH(Me)NH— | H | i-Pr | 527.32 | 527 |
| 12-49 | 4-CF₃ | MeOCH₂CH(Me)NH— | (R)-Me | H | 546.46 | 546 |
| 12-50 | 4-CF₃ | S-MeOCH₂CH(Me)NH— | (R)-Me | H | 546.46 | 546 |
| 12-51 | 4-CF₃ | S-MeOCH₂CH(Me)NH— | S-Me | H | 546.46 | 546 |
| 12-52 | 4-CF₃ | R-MeOCH₂CH(Me)NH— | (R)-Me | H | 546.46 | 546 |
| 12-53 | 4-CF₃ | R-MeOCH₂CH(Me)NH— | S-Me | H | 546.46 | 546 |
| 12-54 | 4-CF₃ | MeOCH₂CH₂N(Me)— | H | H | 532.43 | 533 |
| 12-55 | 4-CF₃ | MeOCH₂CH₂N(Me)— | (R)-Me | NH₂- | 561.47 | 561 |
| 12-56 | 4-CF₃ | MeOCH₂CH₂N(Me)— | (R)-Me | NH(Boc) | 661.59 | 661 |
| 12-57 | 4-CF₃ | MeOCH₂CH₂N(Me)— | (R)-Me | Me | 560.48 | 560 |
| 12-58 | 4-CF₃ | MeOCH₂CH₂N(Me)— | (R)-Me | (R)-4-methylpiperazineCONH— | 687.6 | 687 |
| 12-59 | 4-CF₃ | MeOCH₂CH₂N(Me)— | (R)-Me | (R)-4-ethylpiperazineCONH— | 701.7 | 701 |
| 12-60 | 4-CF₃ | MeOCH₂CH₂N(Me)— | (R)-Me | (R)-4-piperidineCH₂NCONH— | 701.7 | 701 |
| 12-61 | 4-CF₃ | MeOCH₂CH₂N(Me)— | (R)-Me | (R)-4- | 701.7 | 701 |
| | | | | methylhomopiperazineCONH | | |
| 12-62 | 4-CF₃ | MeOCH₂CH₂N(Me)— | (R)-Me | (R)-Me₂NCH₂CONH— | 646.6 | 646 |
| 12-63 | 4-CF₃ | MeOCH₂CH₂N(Me)— | (R)-Me | (R)-Me₂NCH₂CH₂CONH— | 660.6 | 660 |
| 12-64 | 4-CF₃ | MeOCH₂CH₂N(Me)— | (R)-Me | (R)-2-piperidineCONH—672.6 | 672 | |
| 12-65 | 4-CF₃ | MeOCH₂CH₂N(Me)— | (R)-Me | (R)-3-piperidineCONH—672.6 | 672 | |
| 12-66 | 4-CF₃ | MeOCH₂CH₂N(Me)— | (R)-Me | (R)-4-piperidineCONH—672.6 | 672 | |
| 12-67 | 4-CF₃ | MeOCH₂CH₂N(Me)— | (R)-Me | (R)-MeNHCH₂CONH— | 632.6 | 632 |
| 12-68 | 4-CF₃ | MeOCH₂CH₂N(Me)— | (R)-Me | (R)-2-pyrrolidineCONH— | 658.6 | 658 |
| 12-69 | 4-CF₃ | MeOCH₂CH₂N(Me)— | (R)-Me | (R)-3-morpholineCONH—674.6 | 674 | |
| 12-70 | 4-CF₃ | MeOCH₂CH₂N(Me)— | (R)-Me | (R)-NH₂C(Me)₂CONH— | 646.6 | 646 |
| 12-71 | 4-CF₃ | MeOCH₂CH₂N(Me)— | (R)-Me | (R)-NH₂CH₂CH₂CONH— | 632.6 | 632 |
| 12-72 | 4-CF₃ | MeOCH₂CH₂N(Me)— | (S)-Me | (R)-NH₂CH₂CH₂CONH— | 632.6 | 632 |
| 12-73 | 4-CF₃ | MeOCH₂CH(Me)NH— | (R)-Me | (R)-NH₂CH₂CH₂CONH— | 632.6 | 632 |
| 12-74 | 4-CF₃ | MeOCH₂CH(Me)NLi- | (S)-Me | (R)-NH₂CH₂CH₂CONH— | 632.6 | 632 |
| 12-75 | 4-CF₃ | 2-MeOPhCH₂CH₂NH— | (R)-Me | (R)-NH₂CH₂CH₂CONH— | 694.6 | 694 |
| 12-76 | 4-CF₃ | 2-MeOPhCH₂CH₂NH— | (S)-Me | (R)-NH₂CH₂CH₂CONH— | 694.6 | 694 |
| 12-77 | 4-CF₃ | 2-thiophenylCH₂CH₂NH— | (R)-Me | (R)-NH₂CH₂CH₂CONH— | 670.6 | 670 |
| 12-78 | 4-CF₃ | 2-thiophenylCH₂CH₂NH— | (S)-Me | (R)-NH₂CH₂CH₂CONH— | 670.6 | 670 |
| 12-79 | 4-CF₃ | HOCH₂CH(Me)NH— | (R)-Me | (R)-NH₂CH₂CH₂CONH— | 618.5 | 618 |
| 12-80 | 4-CF₃ | HOCH₂CH(Me)NH— | (S)-Me | (R)-NH₂CH₂CH₂CONH— | 618.5 | 618 |
| 12-81 | 4-CF₃ | MeOCH₂CH(Et)NH— | (R)-Me | (R)-NH₂CH₂CH₂CONH— | 646.6 | 646 |
| 12-82 | 4-CF₃ | MeOCH₂CH(Et)NH— | (S)-Me | (R)-NH₂CH₂CH₂CONH— | 646.6 | 646 |
| 12-83 | 4-CF₃ | MeOCOCH₂CH₂NH— | (R)-Me | (R)-NH₂CH₂CH₂CONH— | 646.5 | 646 |
| 12-84 | 4-CF₃ | MeOCOCH₂CH₂NH— | (S)-Me | (R)-NH₂CH₂CH₂CONH— | 646.5 | 646 |
| 12-85 | 4-CF₃ | Et₂N— | (R)-Me | (R)-NH₂CH₂CH₂CONH— | 616.6 | 616 |
| 12-86 | 4-CF₃ | Et₂N— | (S)-Me | (R)-NH₂CH₂CH₂CONH— | 616.6 | 616 |

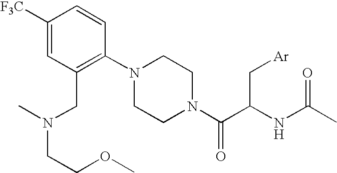

| Cpd | Ar | Mol Wt |
|---|---|---|
| 12-87 | 2-F-phenyl | 538.58 |
| 12-88 | (R)-2-Me-phenyl | 534.62 |
| 12-89 | (R)-3-CN-phenyl | 545.60 |
| 12-90 | 4-F-phenyl | 538.58 |
| 12-91 | 4-Br-phenyl | 599.49 |
| 12-92 | 4-CF₃-phenyl | 588.59 |
| 12-93 | (S)-4-(CF₃)-phenyl | 604.59 |
| 12-94 | (R)-4-(t-Bu)-phenyl | 576.70 |
| 12-95 | (S)-4-(MeO)-phenyl | 550.62 |
| 12-96 | (R)-4-(MeO)-phenyl | 550.62 |
| 12-97 | (R)-4-(EtO)-phenyl | 564.65 |
| 12-98 | (S)-4-(i-PrO)-phenyl | 578.67 |
| 12-99 | (S)-4-(t-BuO)-phenyl | 592.70 |
| 12-100 | (S)-3,4-Me-phenyl | 548.65 |
| 12-101 | (R)-3,4-MeO-phenyl | 580.64 |

### Example 13

### 1-[2-(2-OXO-1-IMIDAZOLIDINYL)-3-(2,4-DICHLOROPHENYL)PROPIONYLI-4-[2-(1-METHYL-2-METHOXYETHYL)AMINOMETHYL)-4-FLUOROPHENYL]PIPERAZINE

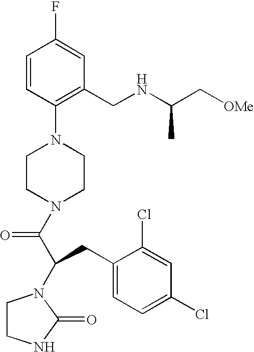

Step 13A:

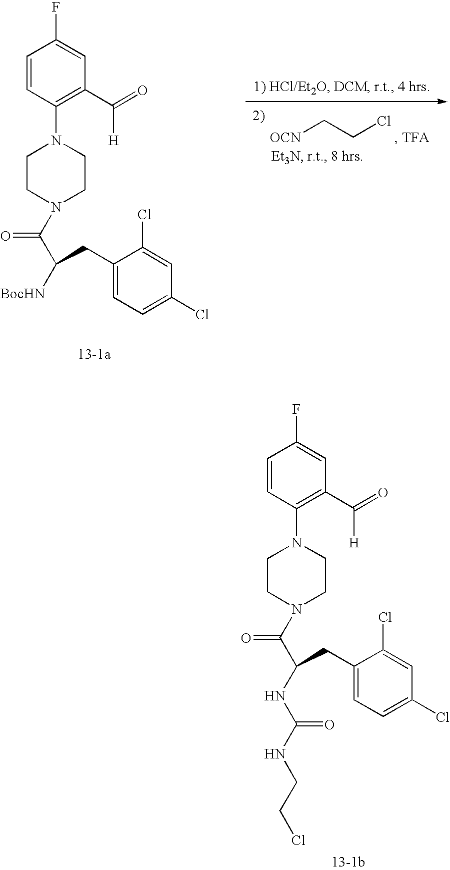

Fluorobenzaldehyde 13-1a (1.25 g, 2.39 mmol) was dissolved in dichloromethane (15 mL) along with 10 mL of 2M HCl in ether solution. The reaction mixture was allowed to stir at room temperature for 4 hours then solvent was removed in vacuo. The deprotected amine was recovered as the HCl salt in 88% yield (0.97 g, 2.1 mmol). This intermediate amine-HCl salt (0.97 g, 2.1 mmol) was then dissolved in THF (8 mL) along with 2-chloroethyl isocyanate (182 uL, 2.l mmol) and Et₃N (585 uL, 4.21 mmol). The reaction mixture was allowed to stir at room temperature for 8 hours then washed with saturated NaHCO₃ solution (3×15 mL) and saturated NaCI solution (15 mL). The organic layer was separated, dried over anhydrous MgSO₄, filtered, and solvent was removed in vacuo. The residue was purified by column chromatography on silica using 50% ethyl acetate/hexanes as the eluent (R_{f}0.3) to give compound 13-1b as an off-white solid in 74% overall yield (0.9 g, 1.77 mmol).

Step 13B:

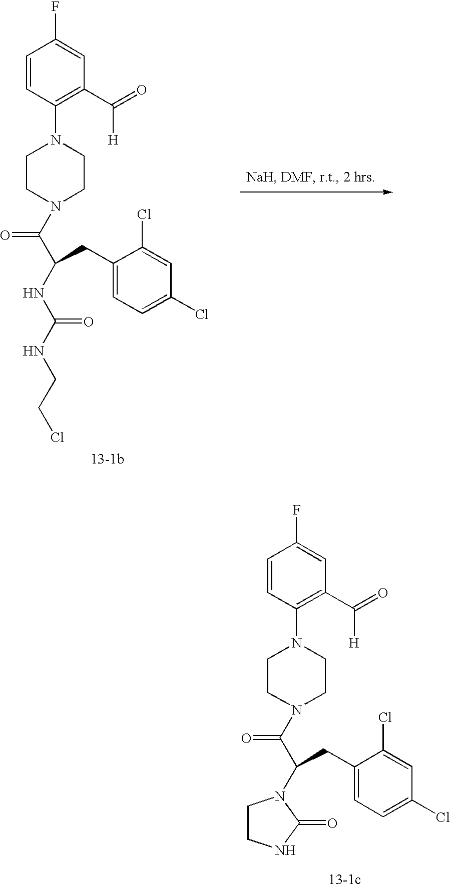

Fluorobenzaldehyde urea 13-1b (0.94 g, 1.77 mmol) was dissolved in DMF (4 mL) and stirred at room temperature. To the reaction mixture, NaH (89 mg, 2.22 mmol) was added in small portions over a period of 30 minutes. After the addition, the reaction mixture was allowed to stir at room temperature for an additional 1.5 hours then was quenched with water (10 mL). The reaction mixture was extracted with ethyl acetate (3×10 mL). The organic layers were combined, dried over anhydrous MgSO₄, filtered, and the solvent was removed in vacuo. The crude product was purified by column chromatography on silica using 85% ethyl acetate/hexanes as the eluent (R_{f}=0.3). The fluorobenzaldehyde cyclic urea 13-1c was recovered as a solid in 55% yield (0.477 g, 0.97 mmol).

Step 13C:

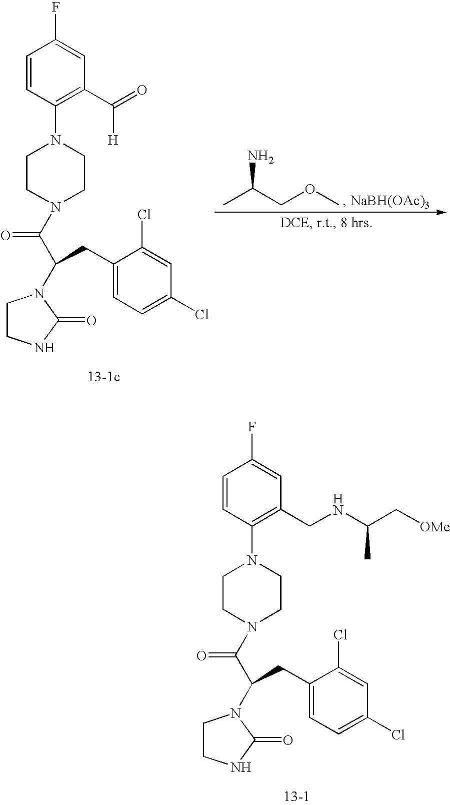

Fluorobenzaldehyde cyclic urea 13-1c (330 mg, 0.66 mmol) was dissolved in dichloroethane (2.5 mL) along with (R)-1-methoxy-2-propyl amine (59 mg, 0.66 mmol). The mixture was allowed to stir at room temperature for 1 hour then NaBH(OAc)₃ (196 mg, 0.93 mmol) was added in one portion. The reaction mixture was allowed to stir at room temperature for 8 hours then quenched with saturated NaHCO₃ (1 mL). The product was extracted with dichloromethane (3×1 mL) and the combined extracts were dried over anhydrous MgSO₄. The mixture was then filtered and solvent was removed in vacuo. The residue was dissolved in MeOH (4 mL) and the product was purified by prep HPLC. The recovered fractions were combined and solvent was removed in vacuo to give the product as the TFA salt. The TFA salt was converted to the HCl salt by dissolving the residue in dichloromethane, washing with saturated NaHCO₃ (2×1 mL), removal of solvent in vacuo, and redissolving in MeOH with HCl in ether. The solvents were then evaporated to give compound 13-1 as the HCl salt in 13% yield (50 mg).

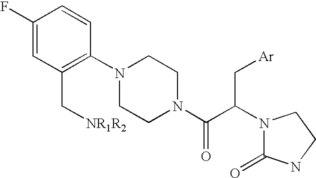

| Cpd | Ar | R₁R₂N— | MS | Mol Wt |
|---|---|---|---|---|
| 13-1 | 4-CF₃-phenyl | MeOCH₂CH₂N(Me)— | 617 | 616.51 |
| 13-2 | 4-CF₃-phenyl | (R)-MeOCH₂CH(Me)NH— | 617 | 616.51 |
| 13-3 | 4-CF₃-phenyl | i-Pr | 586 | 586.48 |
| 13-4 | 4-CF₃-phenyl | 2-F-PhCH₂CH₂NH— | 667 | 666.54 |
| 13-5 | 4-CF₃-phenyl | c-hexyl-NH— | 627 | 626.55 |
| 13-6 | 4-CF₃-phenyl | CH(Me)₂CH(Me)NH— | 615 | 614.54 |
| 13-7 | 4-CF₃-phenyl | c-Pr-CH₂NH— | 598 | 598.49 |
| 13-8 | 4-CF₃-phenyl | MeOCH₂CH₂NH— | 602 | 602.48 |
| 13-9 | 4-CF₃-phenyl | CH₃CH₂C(Me)₂NH— | 615 | 614.54 |
| 13-10 | 4-CF₃-phenyl | CH(Me)₂CH(CH₂OH)NH— | 631 | 630.54 |
| 13-11 | 4-CF₃-phenyl | 2-furanCH₂NH— | 624 | 624.49 |
| 13-12 | 4-CF₃-phenyl | 3-pentylNH— | 615 | 614.54 |
| 13-13 | 4-CF₃-phenyl | n-BuNH— | 601 | 600.51 |
| 13-14 | 4-CF₃-phenyl | s-BuNH— | 601 | 600.51 |
| 13-15 | 4-CF₃-phenyl | CH₃CH₂CH₂CH(Me)NH— | 615 | 614.54 |
| 13-16 | phenyl | 2-thiophenylCH₂CH₂NH— | 586 | 586.59 |
| 13-17 | 4-F-phenyl | (R)-MeOCH₂CH(Me)NH— | 567 | 566.50 |
| 13-18 | 4-F-phenyl | MeOCH₂CH₂N(Me)— | 567 | 566.50 |
| 13-19 | 4-F-phenyl | CH₃CH₂CH₂CH(Me)NH— | 565 | 564.53 |
| 13-20 | 4-F-phenyl | (R)-HOCH₂CH(Me)NH— | 552 | 552.48 |

### Example 14

### 1-[2-(2-OXO-3-{N-PIPERIDINYLETHYL}-1-IMIDAZOLIDINYL)-3-(2,4-DICHLOROPHENYL)PROPIONYL]-4-[2-{N-METHOXYETHYL-N-METHYLAMINO)METHYL}-4-(TRIFLUOROMETHYL)PHENYL]PIPERAZINE

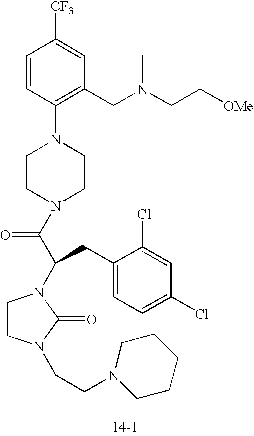

Step 14A:

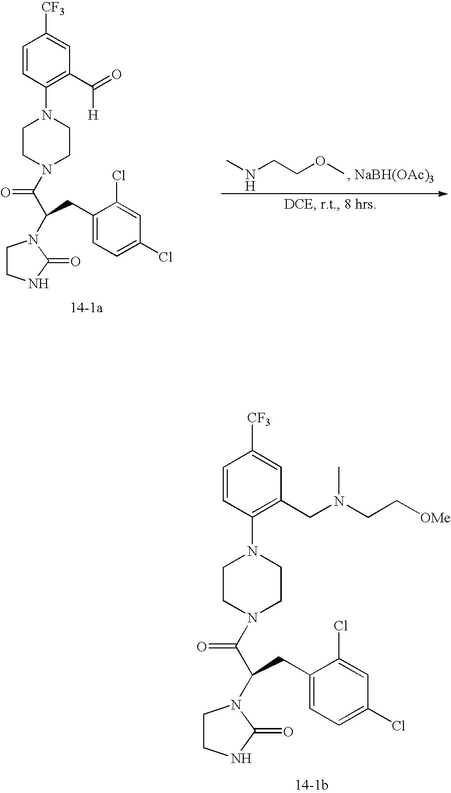

Trifluoromethylbenzaldehyde cyclic urea analog 14-1a (978 mg, 1.8 mmol) was dissolved in dichloroethane (7 mL) along with N-(2-methoxyethyl)methylamine (193 mg, 1.8 mmol). The mixture was allowed to stir at room temperature for 1 hour then NaBH(OAc)₃ (534 mg, 2.5 mmol) was added in one portion. The reaction mixture was allowed to stir at room temperature for 8 hours then was quenched with saturated NaHCO₃ (10 mL). The product was extracted with dichloromethane (3×7 mL) and the combined extracts were dried over anhydrous MgSO₄. The mixture was then filtered and solvent was removed in vacuo. The residue was isolated in 88% yield (981 mg) as a yellow solid without further purification.

Step 14B:

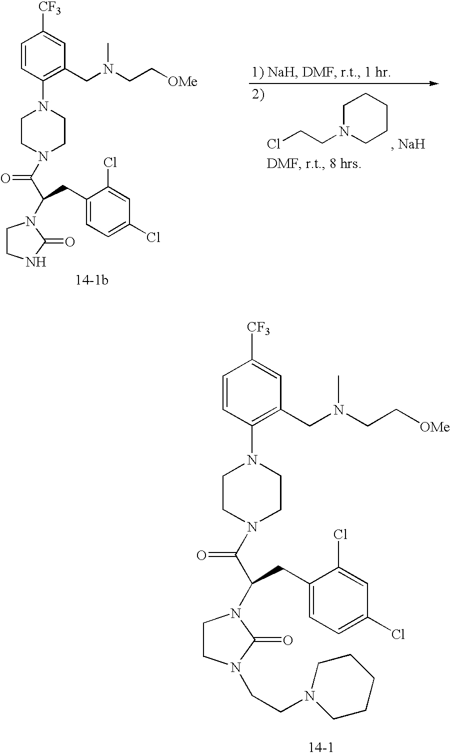

Compound 14-1b (981 mg, 1.6 mmol) was dissolved in DMF (3.2 mL) along with NaH (71 mg, 1.8 mmol). The reaction mixture was allowed to stir at room temperature for 1 hour then 1-(27chloroethyl)piperidine (55 mg, 0.3 mmol) and NaH (13 mg, 0.3 mmol) were added. The reaction mixture was stirred at room temperature for an additional 8 hrs then was quenched with saturated NaHCO₃ (1 mL). The product was extracted with ethyl acetate (3×2 mL). The organic layers were combined, washed with saturated NaCl (5 mL), dried over anhydrous Na₂SO₄, and filtered. The solvent was evaporated under a stream of nitrogen and the residue was redissolved in MeOH. The crude material was purified by prep HPLC to give 14-1 as the TFA salt in 4% yield (6.1 mg).

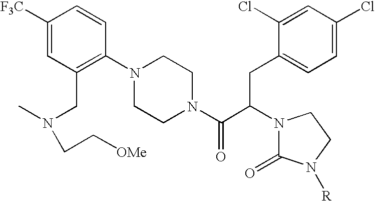

| | R | | |
|---|---|---|---|
| Cpd | (heterocycle substituent) | MS | Mol Wt |
| 14-1 | (1-piperidine)CH₂CH₂— | 728 | 727.70 |
| 14-2 | Me₂NCH₂CH₂— | 688 | 687.63 |
| 14-3 | (1-morpholine)CH₂CH₂— | 730 | 729.67 |

### Example 15

### 1-[1-(2,4-DICHLOROBENZYL)-2-OXO-2-(4-{2-[2-THIOPHEN-2-YL-ETHYLAMINO)METHYL]-PHENYL}PIPERAZIN-1-YL)ETHYL]-OXAZOLIDIN-2-ONE

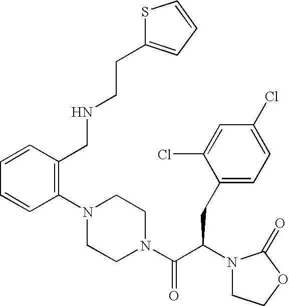

Step 15A:

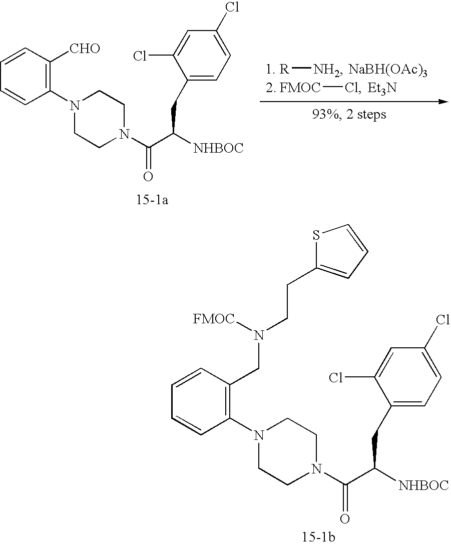

To a solution of the aldehyde 15-1a (2.70 g, 5.35 mmol) and 2-thiopheneethylamine (0.713 g, 5.62 mmol) in dichloromethane (30 mL) was added sodium triacetoxyborohydride (1.59 g, 7.50 mmol). The mixture was stirred overnight, then washed with saturated aqueous sodium bicarbonate solution (15 mL), dried over magnesium sulfate, and evaporated at reduced pressure to give the crude amine as a yellow foam (3.05 g; MS=617.2 (M+H)⁺). A portion of the crude amine (1.22 g, 1.98 mmol) was immediately dissolved in dichloromethane (5 mL) and cooled in an ice-bath. FMOC—Cl (0.51 g, 1.98 mmol) and triethylamine (0.3 mL) were added, and the mixture was stirred for 0.5 h. The mixture was loaded directly onto a silica gel column, and elution with 40% ethyl acetate/hexane provided the FMOC-protected amine 15-1b as a yellow foam (1.61 g, 93%).

Step 15B:

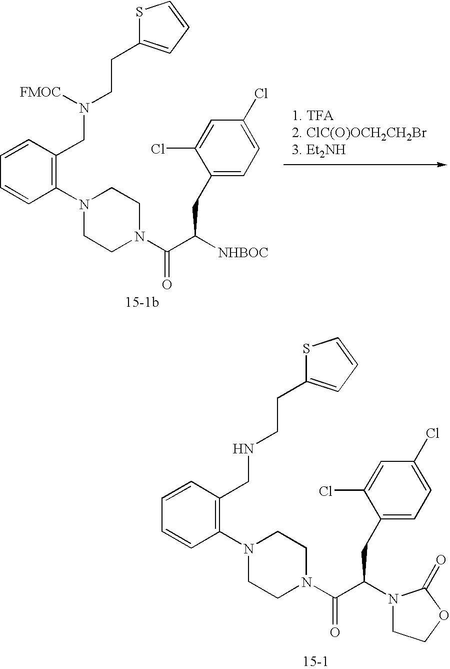

To 15-1b (1.61 g, 1.92 mmol) was added 1:1 dichloromethane/trifluoracetic acid (6 mL). The solution was stirred for 0.5 h, concentrated, dissolved in ethyl acetate (20 mL), washed with saturated aqueous sodium bicarbonate solution (10 mL), dried over magnesium sulfate, and evaporated at reduced pressure to give the crude free base as a yellow foam (1.36 g; MS=739.2 (M+H)⁺). A portion of the amine (30 mg, 0.041 mmol), diisopropylethylamine (13 mg, 0.13 mmol), DMAP (1 crystal), and 2-bromoethyl chloroformate (12 mg, 0.064 mmol) was stirred in dichloromethane (1 mL) overnight. The mixture was diluted with ethyl acetate (5 mL), washed with saturated aqueous sodium bicarbonate solution (2 mL), and dried over magnesium sulfate. The crude was re-dissolved in DMF (0.5 mL), and diisopropylethylamine (2 drops) and lithium iodide (10 mg, 0.075 mmol) were added. The mixture was heated at 100° C. overnight, then evaporated, dissolved in 1:1 diethyl amine/acetonitrile (1 mL), and stirred for 0.5 h. Following evaporation, the crude was purified by preparative LCMS to give compound 15-1 as a yellow oil (8 mg, 32%, 3 steps; MS=599.2 (M+H)⁺).

### Example 16

### 1-[2-(2-OXO-1-PYRROLIDINYL)-3-(2,4-DICHLOROPHENYL)PROPIONYL]-4-[2-(1-METHYLAMINO-2-METHYLBUTYL)-4-TRIFLUOROMETHYLPHENYL]-2-METHYL-PIPERAZINE

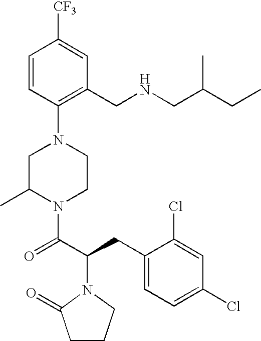

Step 16A:

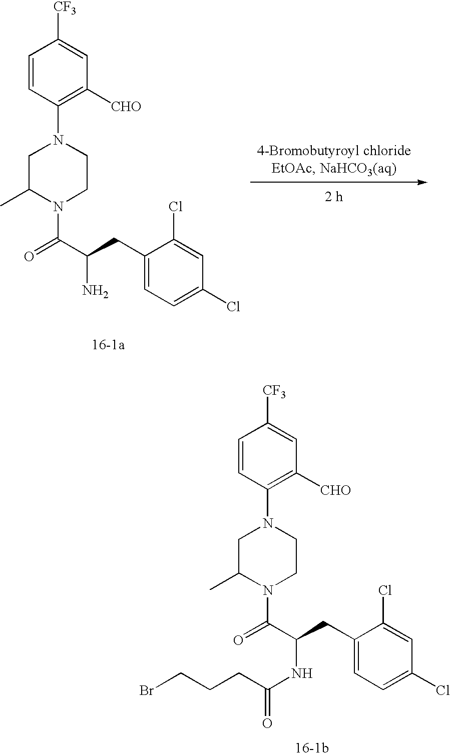

To the solution of 16-1a (700 mg, 1.40 mmol) in EtOAc (7 mL) and sat. NaHCO₃ (7 mL) was added 4-bromobutyroyl chloride (324 μL, 2.80 mmol) dropwise and the reaction was stirred at room temperature for 2 h. The organic layer was separated and the aqueous layer was extracted with EtOAc (20 mL). The combined organic layers were washed with brine, dried over MgSO₄, filtered and concentrated to give compound 16-1b.

Step 16B:

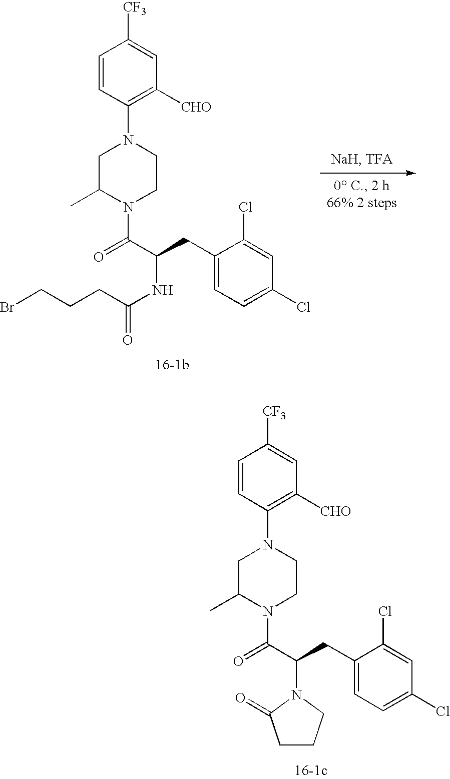

The bromoamide 16-1b was dissolved in 14 mL dry THF and then cooled to 0° C. NaH (56 mg, 60% suspension in mineral oil, 1.40 mmol) was added to the solution. The reaction mix was stirred for 1 h at the same temperature and was quenched by adding sat. NH₄Cl solution (20 mL). The product was extracted with EtOAc (2×20 mL). The organic layer was washed with brine, dried over MgSO₄, filtered and concentrated. The residue was purified by flash column chromatography (Hex: EtOAc 2:1 to 1:2) to afford the lactam 16-1c as a yellow foam (525 mg, 0.92 mmol). The yield was 66% over two steps.

Step 16C:

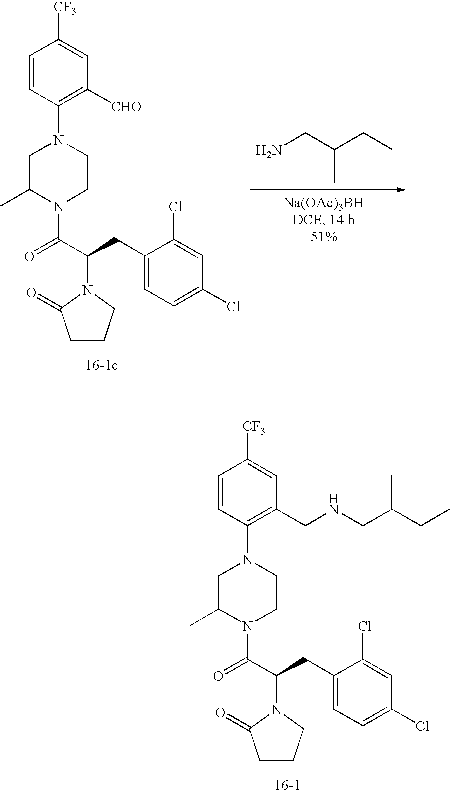

To the solution of lactam 16-1c (50 mg, 0.09 mmol) in 0.4 mL dichloroethane was added 2-methyl-1-butylamine (16 mg, 0.18 mmol). The reaction mix was stirred for 30 minutes, then Na(OAc)₃BH (38 mg, 0.18 mmol) was added. The reaction was allowed to stir at room temperature for 14 h and then was quenched by adding 2 mL H₂O. The product was extracted by dichloromethane (2 mL, twice) and the organic solution was dried over Na₂SO₄, filtered and concentrated. The product 16-1 was purified by HPLC as a TFA salt (39 mg, MW 855.62, 0.046 mmol) in 51% yield.

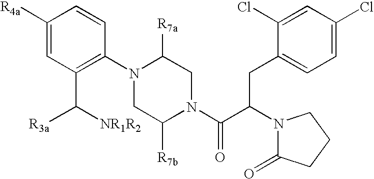

| Cpd | R₄ₐ | R₃ₐ | R₁R₂N— | R₇ₐ | R_{7b} | MS | Mol Wt |
|---|---|---|---|---|---|---|---|
| 16-1 | 4-CF₃-Ph | H | CH₃CH(Et)CH₂NH— | H | Me | 628 | 627.58 |
| 16-2 | 4-CF₃-Ph | H | CH₃CH₂CH₂CH(Me)NH— | Me | Me | 642 | 641.60 |
| 16-3 | 4-CF₃-Ph | H | 2-F-PhCH₂CH₂NH— | Me | Me | 694 | 693.61 |
| 16-4 | 4-CF₃-Ph | H | (R,S)-MeOCH₂CH(Me)NH— | Me | Me | 644 | 643.57 |
| 16-5 | 4-CF₃-Ph | H | 4-Py-CH₂CH₂NH— | Me | Me | 677 | 676.61 |
| 16-6 | 4-CF₃-Ph | H | s-BuNH— | Me | Me | 628 | 627.58 |
| 16-7 | 4-CF₃-Ph | H | MeOCOCH₂CH₂NH— | Me | Me | 658 | 657.56 |
| 16-8 | 4-CF₃-Ph | H | n-BuNH— | Me | Me | 628 | 627.58 |
| 16-9 | 4-CF₃-Ph | H | NCCH₂CH₂NH— | Me | Me | 625 | 624.53 |
| 16-10 | 4-CF₃-Ph | H | 4-Im-CH₂CH₂NH— | Me | Me | 666 | 665.58 |
| 16-11 | 4-CF₃-Ph | H | Me₂NCH₂CH₂CH₂NH— | Me | Me | 657 | 656.62 |
| 16-12 | 4-CF₃-Ph | H | MeOCH₂CH₂N(Me)— | Me | Me | 644 | 643.57 |
| 16-13 | 4-CF₃-Ph | H | (R,S)-HOCH₂CH(Me)NH— | Me | Me | 630 | 629.55 |
| 16-14 | 4-CF₃-Ph | H | CH₃CH₂CH₂CH(Me)NH— | H | Me | 628 | 627.58 |
| 16-15 | 4-CF₃-Ph | H | 2-F-PhCH₂CH₂NH— | H | Me | 680 | 679.58 |
| 16-16 | 4-CF₃-Ph | H | cyclohexylNH— | H | Me | 640 | 639.59 |
| 16-17 | 4-CF₃-Ph | H | (R,S)-MeOCH₂CH(Me)NH— | H | Me | 630 | 629.55 |
| 16-18 | 4-CF₃-Ph | H | (R)-MeOCH₂CH(Me)NH— | H | Me | 630 | 629.55 |
| 16-19 | 4-CF₃-Ph | H | cycloheptylNH— | H | Me | 654 | 653.61 |
| 16-20 | 4-CF₃-Ph | H | 3,4- | H | Me | 692 | 691.58 |
| | | | methylenedioxybenzylNH— | | | | |
| 16-21 | 4-CF₃-Ph | H | 4-Py-CH₂CH₂NH— | H | Me | 663 | 662.58 |
| 16-22 | 4-CF₃-Ph | H | s-BuNH— | H | Me | 614 | 613.55 |
| 16-23 | 4-CF₃-Ph | H | MeOCOCH₂CH₂NH— | H | Me | 644 | 643.53 |
| 16-24 | 4-CF₃-Ph | H | (1-Me-pyrrolidin-2- | H | Me | 669 | 668.63 |
| | | | yl)CH₂CH₂NH— | | | | |
| 16-25 | 4-CF₃-Ph | H | n-BuNH— | H | Me | 614 | 613.55 |
| 16-26 | 4-CF₃-Ph | H | CH₃CH₂C(Me)₂NH— | H | Me | 628 | 627.58 |
| 16-27 | 4-CF₃-Ph | H | NCCH₂CH₂NH— | H | Me | 611 | 610.51 |
| 16-28 | 4-CF₃-Ph | H | i-BuNH— | H | Me | 614 | 613.55 |
| 16-29 | 4-CF₃-Ph | H | 4-Im-CH₂CH₂NH— | H | Me | 652 | 651.56 |
| 16-30 | 4-CF₃-Ph | H | CH(Me)₂CH₂CH(Me)NH— | H | Me | 642 | 641.60 |
| 16-31 | 4-CF₃-Ph | H | Me₂NCH₂CH₂CH₂NH— | H | Me | 643 | 642.59 |
| 16-32 | 4-CF₃-Ph | H | 2-MeOPhCH₂CH₂NH— | H | Me | 692 | 691.62 |
| 16-33 | 4-CF₃-Ph | H | MeOCH₂CH₂N(Me)— | H | Me | 630 | 629.55 |
| 16-34 | 4-CF₃-Ph | H | HOCH₂CH(Me)NH— | H | Me | 616 | 615.52 |
| 16-35 | 4-CF₃-Ph | H | (1-morpholine)CH₂CH₂NH— | H | Me | 671 | 670.60 |
| 16-36 | 4-CF₃-Ph | H | 2-MeBnNH— | H | Me | 662 | 661.59 |
| 16-37 | 4-CF₃-Ph | H | 2-NO₂BnNH— | H | Me | 693 | 692.56 |
| 16-38 | 4-CF₃-Ph | H | MeOCH₂CH₂NH— | H | Me | 616 | 615.52 |
| 16-39 | 4-CF₃-Ph | H | 4-NH₂PhCH₂CH₂NH— | H | Me | 677 | 676.61 |
| 16-40 | 4-CF₃-Ph | H | 4-Me-piperazine | H | Me | 641 | 640.57 |
| 16-41 | 4-CF₃-Ph | H | PhCH₂CH₂N(Me)— | H | Me | 676 | 675.62 |
| 16-42 | 4-CF₃-Ph | H | n-PrN(Me)— | H | Me | 614 | 613.55 |
| 16-43 | 4-CF₃-Ph | H | Et₂N— | H | Me | 614 | 613.55 |
| 16-44 | 4-CF₃-Ph | H | (R)-MeOCH₂CH(Me)NH— | H | H | 616 | 615.52 |
| 16-45 | 4-CF₃-Ph | H | MeOCH₂CH₂N(Me)— | H | H | 616 | 615.52 |
| 16-46 | 4-CF₃-Ph | H | HOCH₂CH₂NH— | H | H | 587 | 587.47 |
| 16-47 | 4-CF₃-Ph | H | 2-MeOBnNH— | H | H | 664 | 663.57 |
| 16-48 | 4-CF₃-Ph | H | CH₃CH(Et)CH₂NH— | H | H | 614 | 613.55 |
| 16-49 | 4-CF₃-Ph | H | (R,S)-MeOCH₂CH(Me)NH— | H | H | 616 | 615.52 |
| 16-50 | 4-CF₃-Ph | H | NCCH₂CH₂NH— | H | H | 596 | 596.48 |
| 16-51 | 4-CF₃-Ph | H | i-BuNH— | H | H | 600 | 599.52 |
| 16-52 | 4-CF₃-Ph | H | HOCH₂CH(Me)NH— | H | H | 601 | 601.49 |
| 16-53 | 4-CF₃-Ph | H | MeOCH₂CH₂NH— | H | H | 601 | 601.49 |
| 16-54 | 4-CF₃-Ph | H | 4-Me-piperazine | H | H | 627 | 626.55 |
| 16-55 | 4-CF₃-Ph | H | n-PrN(Me)— | H | H | 600 | 599.52 |
| 16-56 | 4-CF₃-Ph | H | (4-piperidine)CH₂NH— | H | H | 641 | 640.57 |
| 16-57 | 4-CF₃-Ph | H | (3-pyrrolidine)NH— | H | H | 613 | 612.52 |
| 16-58 | 4-CF₃-Ph | H | 1-piperazine | H | H | 613 | 612.52 |
| 16-59 | 4-CF₃-Ph | H | 4-NH₂-PhCH₂CH₂NH— | H | H | 663 | 662.58 |
| 16-60 | 4-CF₃-Ph | H | n-BuNH— | H | H | 600 | 599.52 |
| 16-61 | 4-F-Ph | H | (R)-MeOCH₂CH(Me)NH— | H | H | 566 | 565.51 |
| 16-62 | Ph | H | (2-thiophenyl)CH₂CH₂NH— | H | H | 586 | 585.60 |
| 16-63 | 6-F-Ph | (S)-i-Bu | NH₂— | H | H | 550 | 549.52 |
| 16-64 | 4-CF₃-Ph | (S)-i-Bu | NH₂— | H | H | 600 | 599.52 |

### Example 17

### 1-[1-(2,4-DICHLOROBENZYL)-2-OXO-2-(4-{2-[2-THIOPHEN-2-YL-ETHYLAMINO)METHYL]-PHENYL}PIPERAZIN-1-YL)ETHYL]PYRROLIDINE-2,5-DIONE

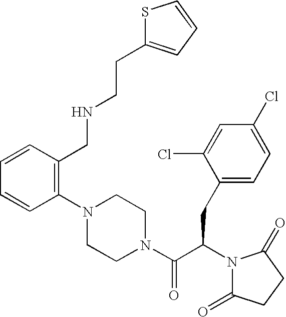

Step 17A:

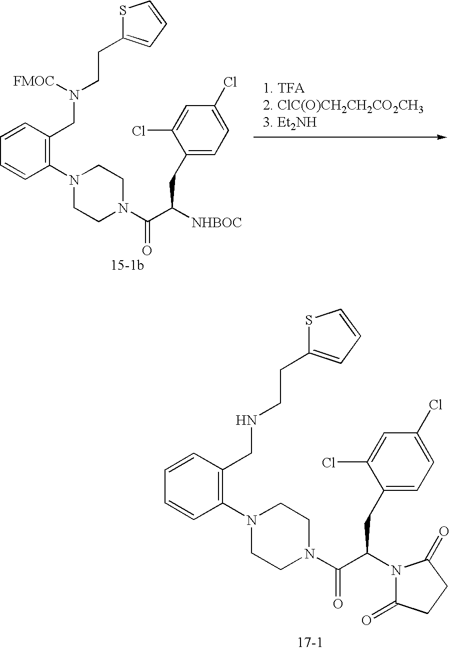

To 15-1b (1.61 g, 1.92 mmol) was added 1:1 dichloromethane/trifluoroacetic acid (6 mL). The solution was stirred for 0.5 h, concentrated, dissolved in ethyl acetate (20 mL), washed with saturated aqueous sodium bicarbonate solution (10 mL), dried over magnesium sulfate, and evaporated at reduced pressure to give a yellow foam (1.36 g; MS=739.2 (M+H)⁺). A portion of the amine (50 mg, 0.068 mmol), diisopropylethylamine (2 drops), and methyl 4-chloro-4-oxobutyrate (11 mg, 0.074 mmol) were stirred in dichloromethane (1 mL) overnight. The mixture was evaporated, re-dissolved in DMF (1 mL), and diisopropylethylamine (2 drops) was added. The mixture was heated at 100° C. overnight. The solvent was evaporated and the residue was dissolved in 1:1 diethyl amine/acetonitrile (1 mL), and stirred for 0.5 h. Following evaporation, the residue was purified by preparative LCMS to give the compound 17-1 as a yellow oil (5 mg, 11% yield for 3 steps; MS=599.2 (M+H)⁺).

### EXAMPLE 18

### 1-[1-(2,4-DICHLORO-BENZYL)-2-(4-{4-FLUORO-2-[(2-METHOXY-1-METHYL-ETHYLAMINO)-METHYL]-PHENYL}-PIPERAZIN-1-YL)-2-OXO-ETHYL]-4-METHYL-PIPERAZIN-2-ONE

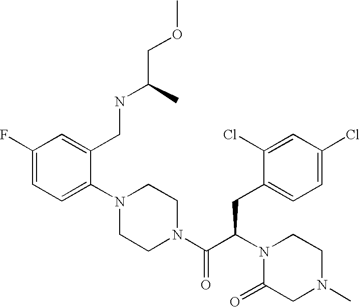

Step 18A: [1-(2,4-Dichloro-benzyl)-2-(4-{4-fluoro-2-[(2-methoxy-1-methyl-ethylamino)-methyl]-phenyl{-piperazin-1-yl)-2-oxo-ethyl]-carbamic acid tert-butyl ester

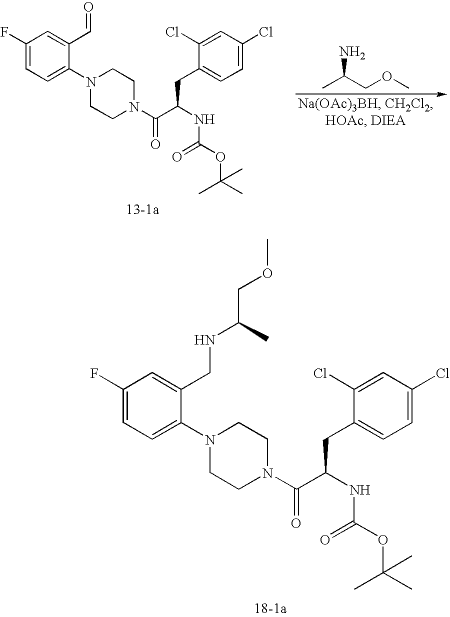

A stirred solution of aldehyde 13-1a ({1-(2,4-dichloro-benzyl)-2-[4-(4-fluoro-2-formyl-phenyl)-piperazin-1-yl]-2-oxo-ethyl}-carbamic acid tert-butyl ester) (1.57 g, 3.0 mmol), (R)-2-methoxy-1-methyl-ethylamine hydrochloride (0.57 g, 4.5 mmol) and diisopropylethylamine (1.6 mL, 9.0 mmol) in dichloromethane (30 mL), at room temperature under N₂, was treated with Na(OAc)₃BH (1.27 g, 6.0 mmol). The resulting suspension was stirred at room temperature for 23 h, and the reaction progress was monitored by LCMS. The reaction mixture was diluted with dichloromethane (100 mL) and was washed with water, aqueous saturated solution of NaHCO₃ and brine. The organic layer was dried over anhydrous MgSO₄, filtered and concentrated in vacuum. Compound 18-1a was obtained as a yellow foam and was used as is in the next step.

Step 18B: {1-(2,4-Dichloro-benzyl)-2-[4-(2-{[(9H-fluoren-9-ylmethoxycarbonyl)-(2-methoxy-1-methyl-ethyl)-amino]-methyl}-4-fluoro-phenyl)-piperazin-1-yl]-2-oxo-ethyl}-carbamic acid tert-butyl ester

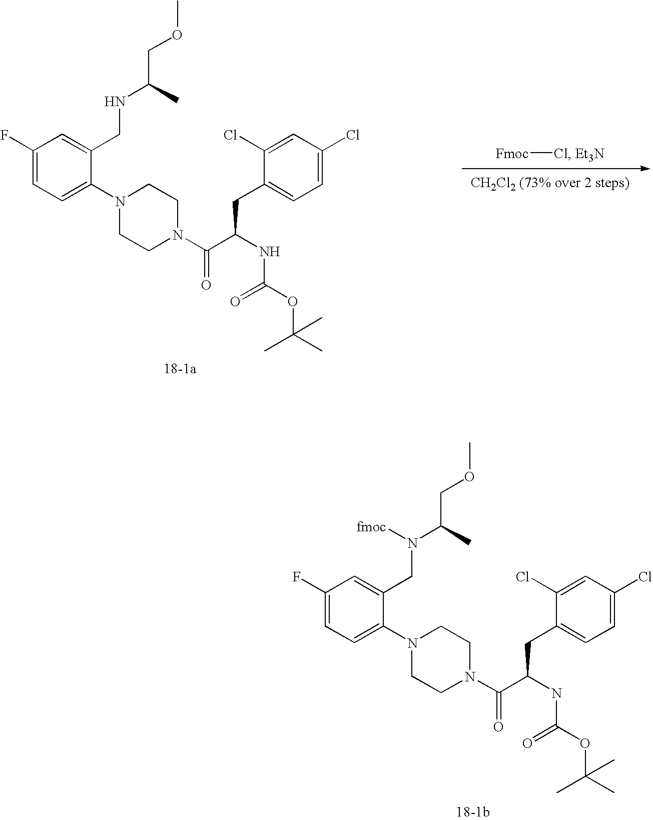

Fmoc chloride (0.93 g, 3.6 mmol) was added portionwise to a stirred solution of compound 18-1a (1.79 g, 3.0 mmol) and triethylamine (0.85 mL, 6.0 mmol) in dichloromethane (15 mL), under N₂. The resulting mixture was stirred at room temperature for 3 h, diluted with EtOAc (100 mL) and washed with water, 1 N HCl and brine. The organic layer was dried over anhydrous MgSO₄, filtered and concentrated in vacuum. Purification by column chromatography on silica-gel, eluting with a 2:1 v/v mixture of hexanes and EtOAc, gave 18-1b as a pale yellow foam (1.78 g, 2.2 mmol, 73%). LCMS m/z 819.6 (M⁺+1).

Step 18C: (2-{1-(2,4-Dichloro-benzyl)-2-[4-(2-{[(9H-fluoren-9-ylmethoxycarbonyl)-(2-methoxy-1-methyl-ethyl)-amino]-methyl}-4-fluoro-phenyl)-piperazin-1-yl]-2-oxo-ethylamino}-ethyl)-carbamic acid tert-butyl ester

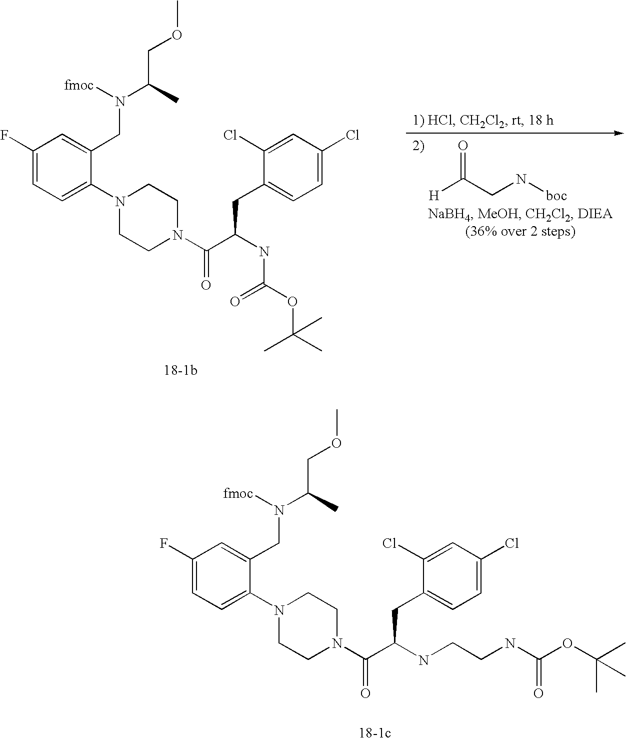

Compound 18-1b (1.78 g, 2.2 mmol) was dissolved in dichloromethane (11 mL) and treated with HCl (2.8 mL of a 4.0 M solution in dioxane, 10.9 mmol). The resulting mixture was stirred at room temperature for 18 h then was concentrated under vacuum to give the crude amine hydrochloride salt as a yellow foam. This foam was dissolved in MeOH (11 mL) and dichloromethane (11 mL) and was treated with diisopropylethylamine (0.8 mL, 4.4 mmol). tert-Butyl N-(2-oxoethyl)carbamate (1.0 g, 6.3 mmol) was then added and the resulting mixture was stirred at room temperature for 1 h. NaBH₄ (0.25 g, 6.5 mmol) was then added portionwise over 15 minutes and the resulting mixture was stirred for 1 h. Another portion of tert-butyl N-(2-oxoethyl)carbamate (1.0 g, 6.3 mmol) was added, followed by more NaBH₄ (0.25 g, 6.5 mmol). The mixture was stirred at room temperature overnight and then worked-up. The crude residue was purified by column chromatography on silica gel, eluting with a 95:5 v/v mixture of EtOAc and MeOH. Compound 18-1c was isolated as a white foam (0.67 g, 0.78 mmol, 36%). LCMS m/z 862.2 (M⁺+1).

Step 18D: (2-{4-[3-(2,4-Dichloro-phenyl)-2-(2-oxo-piperazin-1-yl)-propionyl]-piperazin-1-yl}-5-fluoro-benzyl)-(2-methoxy-1-methyl-ethyl)-carbamic acid9H-fluoren-9-yl methyl ester

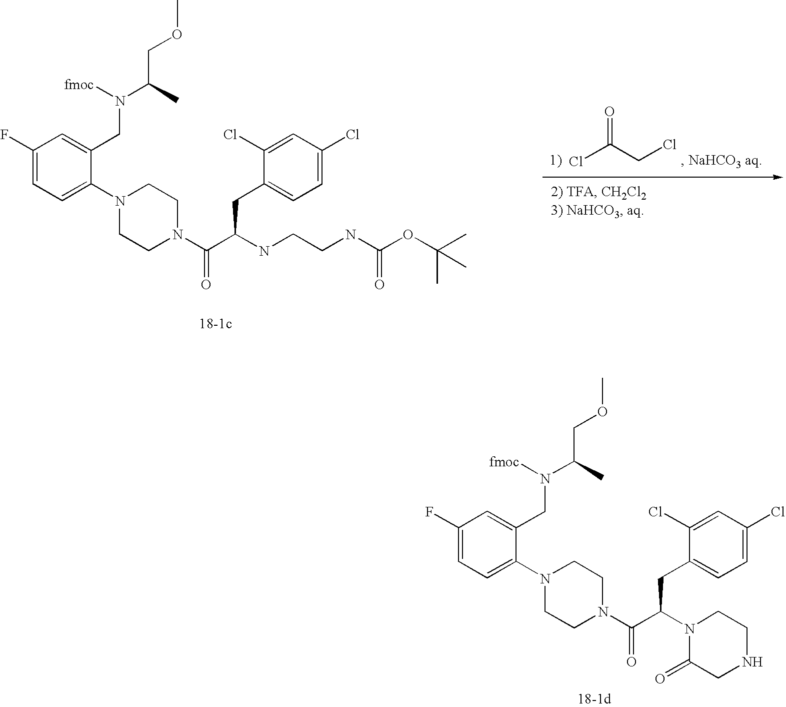

Chloroacetyl chloride (0.13 mL, 1.2 mmol) was added to a vigorously stirring suspension of amine 18-1c (0.52 g, 0.6 mmol) in EtOAc (4 mL) and aqueous saturated NaHCO₃ (4 mL). After 1.5 h, the organic layer was separated and concentrated under vaccum to give a white foam. This foam was treated with a 1:1 v/v solution of dichloromethane and trifluoroacetic acid for 1 h at room temperature. The volatiles were removed under vacuum and the residue was dissolved in dichloromethane (50 mL) and washed with aqueous saturated NaHCO₃ and brine. The organic layer was dried over anhydrous MgSO₄, filtered and concentrated under vacuum. Compound 18-1d was obtained as a yellow foam (0.43 g, 0.53 mmol, 89%). LCMS m/z 802.2 (M⁺+1).

Step 18E: 1-[1-(2,4-Dichloro-benzyl)-2-(4-{4-fluoro-2-[(2-methoxy-1-methyl-ethylamino)-methyl]-phenyl}-piperazin-1-yl)-2-oxo-ethyl]-piperazin-2-one

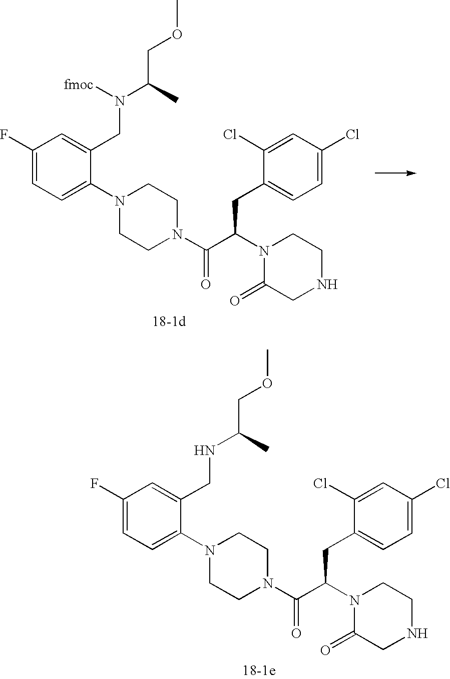

Compound 18-1d (50 mg, 0.06 mmol) was dissolved in a 1:1 v/v mixture of acetonitrile and diethylamine at room temperature. After 2 h, the volatiles were removed in vacuum and the residue was purified by preparative HPLC/MS to give compound 18-1e (20 mg, 0.035 mmol, 56%). LCMS m/z 580.1 (M⁺+1).

Step 18F: 1-[1-(2,4-Dichloro-benzyl)-2-(4-{4-fluoro-2-[(2-methoxy-1-methyl-ethylamino)-methyl]-phenyl{-piperazin-1-yl)-2-oxo-ethyl]-4-methyl-piperazin-2-one

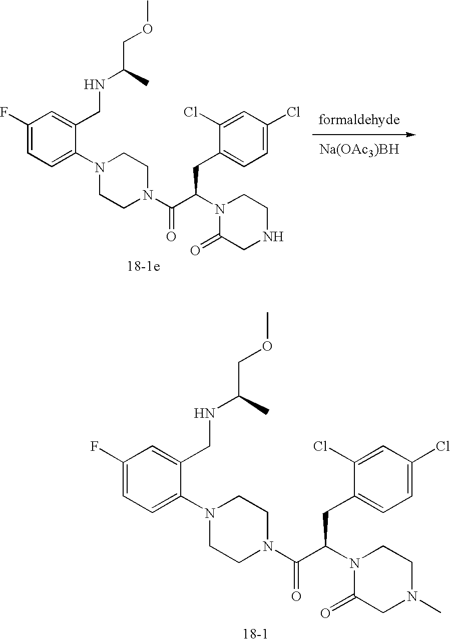

Compound 18-1e (50 mg, 0.06 mmol) was dissolved in dichloromethane (1 mL), treated with formaldehyde (0.5 mL of a 37% wt. aqueous solution) and Na(OAc)₃BH (40 mg, 0.19 mmol) and was stirred at room temperature for 4 h. The volatiles were removed under vacuum and the residue was treated with a 1:1 v/v mixture of acetonitrile and diethylamine (2 mL) for 1 h. The volatiles were evaporated and the residue was dissolved in MeOH (1 mL), filtered and purified by preparative HPLC/MS to give compound 18-1 (22 mg, 0.037 mmol, 60% yield). LCMS m/z 594.2 (M⁺+1).

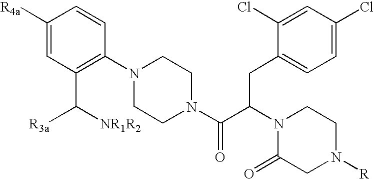

| | | | | R | |
|---|---|---|---|---|---|
| | | | | (heterocycle | |
| Cpd | R₃ₐ | R₁R₂N— | R₄ₐ | substituent) | Mol Wt |
| 18-1 | H | (R)-MeOCH₂CH(Me)NH— | 4-F | Me | 594.56 |
| 18-2 | H | (R)-MeOCH₂CH(Me)NH— | 4-F | H | 580.53 |
| 18-3 | H | (R)-MeOCH₂CH(Me)NH— | 4-F | Et | 608.58 |
| 18-4 | H | (R)-MeOCH₂CH(Me)NH— | 4-F | i-Pr | 622.61 |
| 18-5 | H | (R)-MeOCH₂CH(Me)NH— | 4-F | c-Pr | 620.59 |
| 18-6 | H | MeOCH₂CH₂N(Me)— | 4-CF₃ | H | 630.54 |
| 18-7 | H | MeOCH₂CH₂N(Me)— | 4-CF₃ | Me | 644.56 |
| 18-8 | H | MeOCH₂CH₂N(Me)— | 4-CF₃ | Et | 658.59 |
| 18-9 | H | MeOCH₂CH₂N(Me)— | 4-CF₃ | i-Pr | 672.62 |
| 18-10 | H | MeOCH₂CH₂N(Me)— | 4-CF₃ | c-Pr | 670.60 |
| 18-11 | (S)-i-Bu | NH₂— | 4-CF₃ | H | 614.54 |

### Example 19

### 1-[2-(2-OXO-3-AMINO-1-PYRROLIDINYL)-3-(2,4-DICHLOROPHENYL)PROPIONYL]-4-[2-(1-AMINO-3-METHYLBUTYL)-4-(TRIFLUOROMETHYL)PHENYL]PIPERAZINE

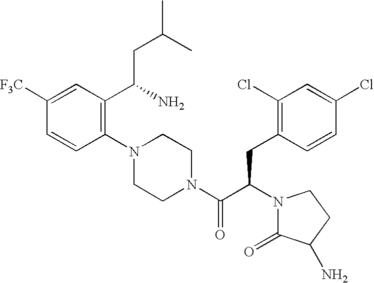

Step 19A:

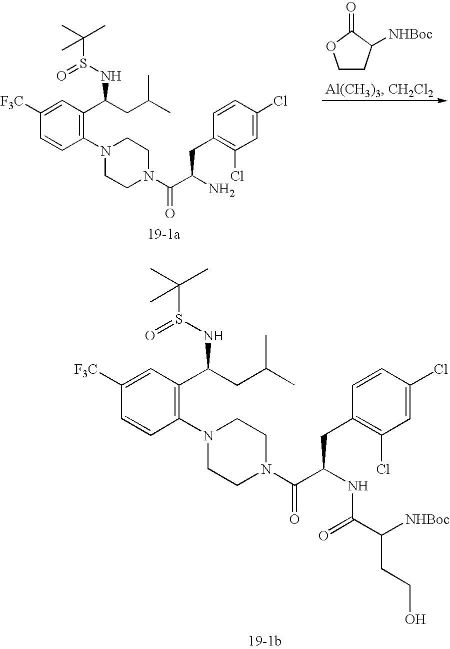

To a mixture of sulfinamide 19-1a (98 mg, 0.16 mmol) in dry methylene chloride (2 mL) under nitrogen, was added trimethylaluminium (0.17 mL, 0.33 mmol) dropwise at room temperature. The reaction mixture was then allowed to stir for 15 minutes and a solution of tert-butyl (tetrahydro-2-oxo-3-furanyl)carbamate (32 mg, 0.16 mmol) dissolved in dry methylene chloride (2 mL) was then added dropwise to the reaction at room temperature and stirred overnight. The mixture was quenched with 4 mL of 10% citric acid, partitioned between methylene chloride and potassium sodium tartrate. The organic layer was separated, dried over magnesium sulfate and then the solvent was removed in vacuo to obtain 19-1b as a white foam (159 mg). LCMS m/z 836.2 (M⁺+H⁺).

Step 19B:

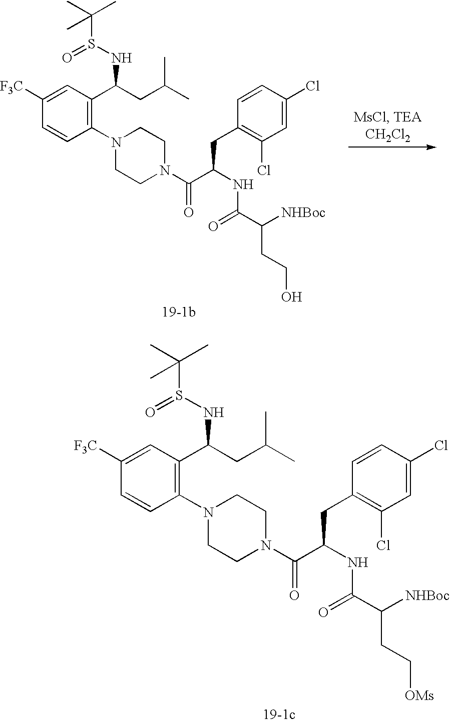

To a mixture of 19-1b (159 mg, 0.19 mmol) in dry methylene chloride (5 mL) was added triethylamine (55 uL, 0.38 mmol) and methanesulfonyl chloride (15 uL 0.19 mmol) at 0° C. The mix was allowed to stir for 2 hours, gradually warming to room temperature. The reaction was then partitioned between methylene chloride and sodium bicarbonate. The organic layer was separated, dried over magnesium sulfate, and removed in vacuo to obtain the mesylate 19-1c as a white foam (163 mg). LCMS m/z 914.3 (M⁺+H⁺).

Step 19C:

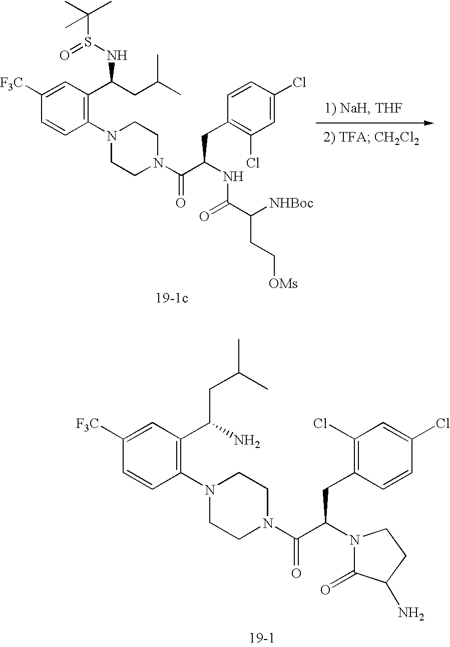

To a mixture of mesylate 19-1c (163 mg, 0.18 mmol) in tetrahydrafuran (10 mL) was added sodium hydride (22 mg, 0.54 mmol). The reaction mix was stirred overnight, and then partitioned between methylene chloride and saturated ammonium chloride. The organic layer was separated, dried over magnesium sulfate and removed in vacuo to yield the protected intermediate. Trifluoroacetic acid (2 mL) and methylene chloride (2 mL) were added to 46 mg 0.06 mmol of the protected intermediate and the reaction was stirred at room temperature for forty-five minutes. The solvent was then removed in vacuo to give a residue which was purified by preparative liquid chromatography to give 19-1 as clear oil (35 mg). LCMS m/z 614.0 (M⁺+H⁺).

### Example 20

### 1-[3-(2,4-DICHLOROPHENYL)PROPIONYL]-4-(3-DIETHYLAMINOMETHYL-2-PYRIDYL)PIPERAZINE

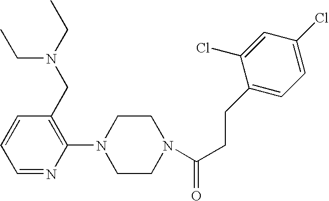

Step 20A: 2-Bromo-3-formylpyridine

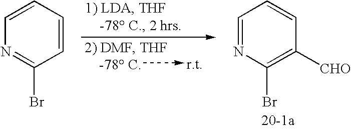

Lithium diisopropylamide (131 mL, 262 mmol, 2M in THF) was added to a stirring solution of 2-bromopyridine (25 mL, 262 mmol) in THF (208 mL) at −78° C. under nitrogen. The reaction mixture was allowed to stir at −78° C. for 2 hours then a solution of DMF (20.3 mL, 262 mmol) in THF (104 mL) was added. After the addition, the reaction mixture was allowed to warm to r.t. and was neutralized by adding to a saturated solution of ammonium chloride. After extraction with ethyl acetate (3×200 mL), the organic layers were combined, dried over anhydrous Na₂SO₄, filtered, and the solvent removed in vacuo. The residue was purified by column chromatography on silica using 15% ethyl acetate/hexanes as the eluent (R_{f}=0.3) to give compound 20-1a in 19% yield as a yellow oil (9.4 g, 50.5 mmol).

Step 20B: Boc-piperazine formylpyridine

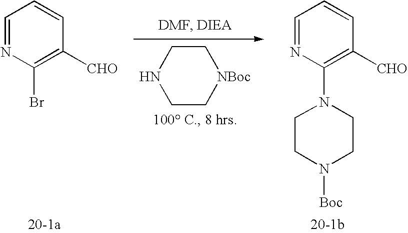

2-Bromo-3-formylpyridine 20-1a (9.4 g, 50.5 mmol) was dissolved in DMF (100 mL) along with diisopropylethylamine (8.8 mL, 50.5 mmol) and 1-Boc-piperazine (9.4 g, 50.5 mmol). The reaction mixture was heated at 100° C. for 8 hours then cooled to room temperature and quenched with saturated NaHCO₃ (150 mL). The crude product was extracted with ethyl acetate (3×100 mL), the organic layers were combined, dried over anhydrous Na₂SO₄, filtered, and solvent removed in vacuo. The residue was purified by column chromatography on silica using 25% ethyl acetate/hexanes as the eluent (R_{f}=0.3) to give 20-1b in 67% yield as a yellow solid (9.8 g, 33.5 mmol).

Step 20C: 1-[3-(2,4-Dichlorophenyl)propionyl]-4-3-formyl-2-pyridylpiperazine

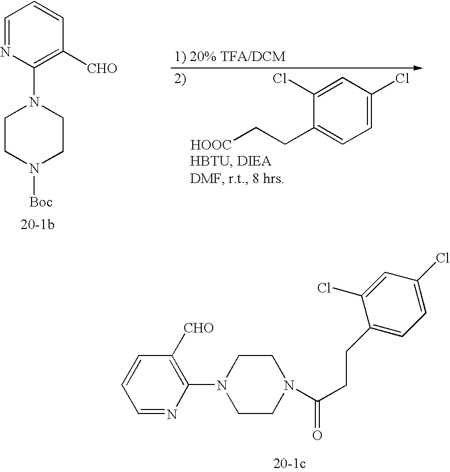

Boc-piperazine formylpyridine 20-1b (2.15 g, 7.4 mmol) was allowed to stir at room temperature for 1 hour in a (1:1) TFA/DCM mixture. The reaction mixture was then concentrated under vacuum and diluted in dichloromethane (30 mL). The organic layer was washed with saturated NaHCO₃ solution (3×50 mL), saturated NaCl solution (50 mL), dried over anhydrous MgSO₄, filtered, and solvent removed in vacuo. This deprotected piperazine intermediate (1.4 g, 7.38 mmol) was added to a solution of 3-(2,4-dichlorophenyl)propionic acid and diisopropylethylamine (2 mL, 14.76 mmol) in DMF (14 mL) that had been stirring under nitrogen atmosphere with HBTU (2.8 g, 7.38 mol) at room temperature for 1 hour. The reaction mixture was allowed to stir for an additional 8 hours at room temperature then diluted with saturated NaHCO₃ solution (50 mL). The crude product was extracted with ethyl acetate (3×75 mL), the organic layers were combined, dried over anhydrous Na₂SO₄, filtered, and solvent removed in vacuo. The residue was purified by column chromatography on silica using 50% ethyl acetate/hexanes as the eluent (R_{f}=0.3). Compound 20-1c was recovered in quantitative yield as a yellow oil (2.9 g, 7.4 mmol).

Step 20D: 1-[3-(2,4-dichlorophenyl)propionyl]-4-diethylaminomethyl-2-pyridylpiperazine

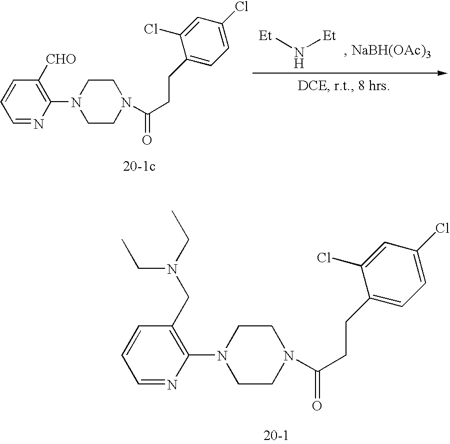

Formylpyridine 20-1c (39.2 mg, 0.1 mmol) was dissolved in DCE (0.5 mL) along with diethylamine (10.3 uL, 0.1 mmol) and stirred for 30 minutes at room temperature. NaHB(OAc)₃ (42 mg, 0.2 mmol) was added and reaction mixture was allowed to stir at room temperature for an additional 8 hours. The reaction mixture was then diluted with dichloromethane (1 mL) and quenched with saturated NaHCO₃ (1 mL). The product was extracted with dichloromethane (3×1 mL) and the combined extracts were dried over anhydrous MgSO₄. The mixture was then filtered and solvent was removed in vacuo. The crude product was purified by prep HPLC to yield compound 20-1 in 33% yield as the TFA salt (18.4 mg, 0.033 mmol).

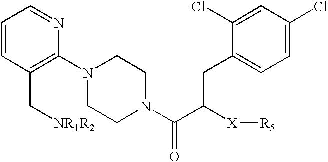

| Cpd | R₅X- | R₁R₂N— | MS | Mol Wt |
|---|---|---|---|---|
| 20-1 | H | Et₂N— | 449 | 449.42 |
| 20-2 | H | EtCH(Me)CH₂NH— | 463 | 463.45 |
| 20-3 | H | PrCH(Me)NH— | 463 | 463.45 |
| 20-4 | H | 2-FPhCH₂CH₂NH— | 515 | 515.46 |
| 20-5 | H | MeOCH₂CH(Me)NH— | 465 | 465.42 |
| 20-6 | H | EtCH(Me)NH— | 449 | 449.42 |
| 20-7 | H | n-BuNH— | 449 | 449.42 |
| 20-8 | H | EtC(Me)₂NH— | 463 | 463.45 |
| 20-9 | H | i-BuNH— | 449 | 449.42 |
| 20-10 | H | CH(Me)₂CH₂CH(Me)NH— | 477 | 477.48 |
| 20-11 | H | MeOCH₂CH₂N(Me)— | 465 | 465.42 |
| 20-12 | H | CycloheptylNH— | 489 | 489.49 |
| 20-13 | H | HOCH₂CH₂NH— | 437 | 437.37 |
| 20-14 | Me | MeOCH₂CH(Me)NH— | 479 | 479.47 |
| 20-15 | (R)-AcNH— | 2-MeOPhCH₂CH₂NH— | 585 | 584.54 |
| 20-16 | (R)-AcNH— | 2-FPhCH₂CH₂NH— | 572 | 572.51 |
| 20-17 | (R)-AcNH— | 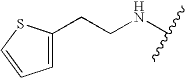 | 561 | 560.55 |
| 20-18 | (R)-AcNH— | MeOCH₂CH(Me)NH— | 522 | 522.47 |
| 20-19 | (R)-NH₂CH₂CH₂CONH— | 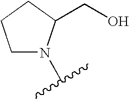 | 563 | 563.53 |
| 20-20 | (R)-NH₂CH₂CH₂CONH— | HOCH₂C(Me)₂NH— | 551 | 551.52 |
| 20-21 | (R)-NH₂CH₂CH₂CONH— | MeOCH₂CH(Me)NH— | 551 | 551.52 |
| 20-22 | (R)-NH₂CH₂CH₂CONH— | HOCH₂CH(Me)NH— | 537 | 537.49 |
| 20-23 | (R)-NH₂CH₂CH₂CONH— | HOCH₂CH(Et)NH— | 551 | 551.52 |
| 20-24 | (R)-NH₂CH₂CH₂CONH— | 2-F-BnNH— | 587 | 587.52 |
| 20-25 | (R)-NH₂CH₂CH₂CONH | CF₃CH₂NH— | 561 | 561.43 |
| 20-26 | (R)-NH₂CH₂CH₂CONH— | HOCH₂CH₂NH— | 523 | 523.46 |
| 20-27 | (R)-NH₂CH₂CH₂CONH— | 2-MeOPhCH₂CH₂NH— | 613 | 613.59 |
| 20-28 | (R)-NH₂CH₂CH₂CONH— | 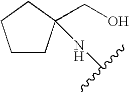 | 577 | 577.55 |
| 20-29 | (R)-NH₂CH₂CH₂CONH— | MeOCH₂CH(Et)NH— | 565 | 565.54 |
| 20-30 | (R)-NH₂CH₂CH₂CONH— | HOCH₂CH(CH₂OH)NH— | 553 | 553.49 |
| 20-31 | (R)-NH₂CH₂CH₂CONH— | 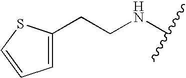 | 589 | 589.59 |
| 20-32 | (R)-NH₂CH₂CH₂CONH— | 2-FPhCH₂CH₂NH— | 601 | 601.55 |

### Example 21

### 1-[3-(2,4-DICHLOROPHENYL)PROPIONYL]-4-(3-[1-AMINO-3-METHYLBUTYL]-2-PYRIDYL)PIPERAZINE

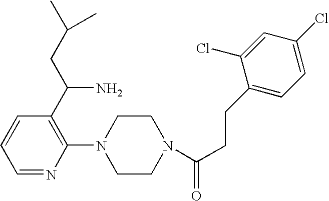

Step 21A:

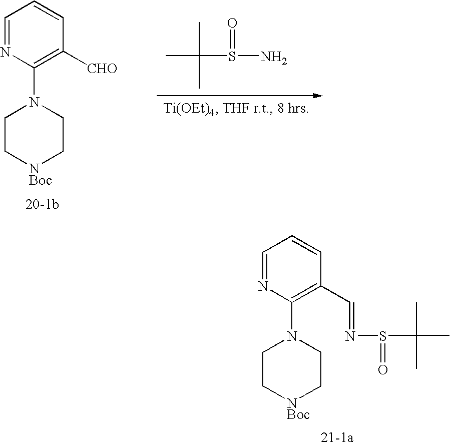

Boc-piperazine formylpyridine 20-1b (3 g, 10.3 mmol) was dissolved in THF (51 mL) along with 2-methyl-2-propanesulfinamide (1.4 g, 11.3 mmol) and titanium (IV) ethoxide (8.6 mL, 41.2 mmol). The reaction mixture was stirred at room temperature for 8 hours then saturated NaCl solution (20 mL) was added. The reaction mixture was filtered and the solid was washed with ethyl acetate (3×75 mL). The organic layer was collected, dried over anhydrous Na₂SO₄, filtered, and solvent removed in vacuo. Compound 21-1a was isolated as a yellow solid in quantitative yield without further purification (4.1 g, 10.3 mmol).

Step 21B:

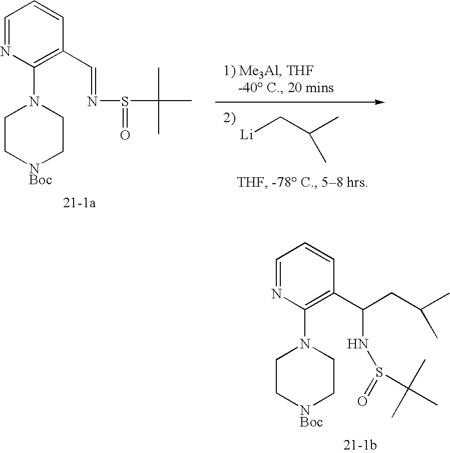

Sulfinyl imine-pyridine 21-1a (4.1 g, 10.3 mmol) in THF (30 mL) was cooled to 40° C. and Me₃Al (15.45 mL, 30.9 mmol) was added. The reaction mixture was allowed to stir at −40° C. under nitrogen atmosphere for 20 minutes then was cooled to −78° C. To the reaction mixture, isobutyl lithium (12.9 mL, 20.6 mmol, 1.6 M inheptane) was added slowly at −78° C. After the addition was complete, the reaction was warmed to room temperature and carefully quenched with water. The crude product mixture was then concentrated under vacuum and diluted with dichloromethane (150 mL). The organic layer was then washed with saturated NaHCO₃ solution (2×100 mL), saturated NaCl solution (100 mL), dried over anhydrous MgSO₄, filtered, and solvent removed in vacuo. The residue was purified by column chromatography on silica using 75% ethyl acetate/hexanes as the eluent (R_{f}=0.3). Compound 21-1b was recovered in 60% yield as a yellow solid (2.8 g, 6.15 mmol).

Step 21C:

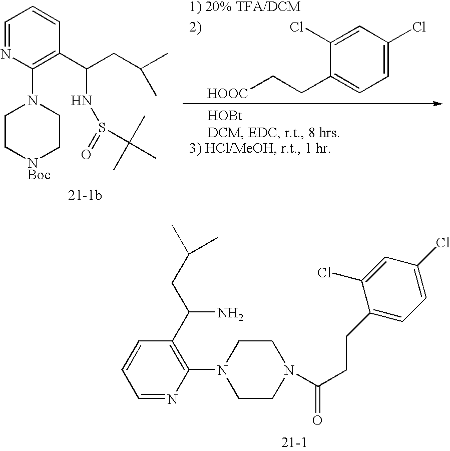

Sulfinamide-pyridine 21-1b (452.6 mg, 1 mmol) was stirred at room temperature for 1.5 hours in 20% TFA/DCM mixture. The reaction was quenched with saturated NaHCO₃ solution (5 mL). The organic layer was washed with saturated NaHCO₃ solution (2×10 mL), saturated NaCl solution (10 mL), dried over anhydrous MgSO₄, filtered, and solvent removed in vacuo. The deprotected piperazine intermediate was recovered in quantitative yield. A small portion of this piperazine intermediate (35.2 mg, 0.1 mmol) was dissolved in dichloromethane (0.5 mL) along with HOBt (13.5 mg, 0.1 mmol) and 3-(2,4-dichlorophenyl)propionic acid (21.9 mg, 0.1 mmol). The reaction mixture was allowed to stir at room temperature for 10 minutes then EDC (19.2 mg, 0.1 mmol) was added. The reaction was then stirred for an additional 8 hours at room temperature followed by quenching with saturated NaHCO₃ solution. The organic layer was separated, washed with saturated NaCl solution (2 mL), dried over anhydrous MgSO₄, filtered, and solvent removed in vacuo. The resulting residue was dissolved in MeOH (2 mL) and 0.2M HCl/ether (1 mL) was added. The reaction was stirred at room temperature for 1 hour then solvent was removed under a stream of nitrogen. The crude product was purified by prep HPLC to yield compound 21-1 in 26% yield as the TFA salt (15 mg, 0.026 mmol).

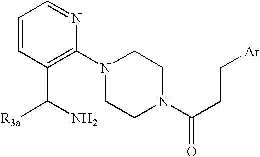

| Cpd | R₃ₐ | —Ar | MS | Mol Wt |
|---|---|---|---|---|
| 21-1 | (R)-i-Bu | 2,4-Cl-phenyl | 449 | 449.42 |
| 21-2 | (S)-i-Bu | 4-Cl-phenyl | 415 | 414.98 |
| 21-3 | (S)-i-Bu | 2,4-Cl-phenyl | 449 | 449.42 |

It will be appreciated that, although specific embodiments of the invention have been described herein for purposes of illustration, various modifications may be made without departing from the spirit and scope of the invention. Accordingly, the invention is not limited except as by the appended claims.

1. A compound having the following structure: 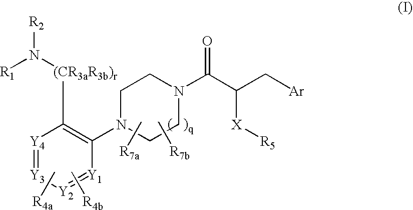
or a stereoisomer, prodrug or pharmaceutically acceptable salt thereof,
wherein:
q is 1 or 2;
r is 1, 2, or 3;
Y₁, Y₂, Y₃ and Y₄ are independently CH or N, with the proviso that no more than two of Y₁, Y₂, Y₃ and Y₄ are N, and with the further proviso that, when two of Y₁, Y₂, Y₃ and Y₄ are N, either Y₁ and Y₃ are N or Y₂ and Y₄ are N;
Ar is phenyl, substituted phenyl, naphthyl, or substituted naphthyl;
X is a bond, —O—, —S—, —N(R₆ₐ)—, —N(R₆ₐ)C(═O)—, —N(R₆ₐ)S(═O)₂—, —N(R₆ₐ)C(═O)N(R_{6b})——C(═O)O—, —OC(═O)—, —N(R₆ₐ)C(═O)N(R_{6b})O—, —N(R₆ₐ)C(═O)N(R_{6b})N(R_{6c})—, or —N(R₆ₐ)C(═O)O—;
R₁ and R₂ are the same or different and independently hydrogen, alkyl, substituted alkyl, aryl; substituted aryl, arylalkyl, substituted arylalkyl, heterocycle, substituted heterocycle, heterocyclealkyl, or substituted heterocyclealkyl;
R₃ₐ and R_{3b} are, at each occurrence, the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₄ₐ and R_{4b} are optional ring substituents and, when one or both are present, are the same or different and independently hydroxy, alkyl, substituted alkyl, cyano, halogen, alkoxy, or alkylamino;
R₅ is hydrogen, alkyl, substituted alkyl, aryl, substituted aryl, heterocycle, or substituted heterocycle;
R₆ₐ, R_{6b} and R_{6c}, are, at each occurrence, the same or different and independently hydrogen, alkyl, or substituted alkyl; and
R₇ₐ and R_{7b} are optional ring substituents and, when one or both are present, are the same or different and independently hydrogen, lower alkyl, or substituted lower alkyl;
with the proviso that when r is 1 then R₁, R₂, R₃ₐ and R_{3b} are not all hydrogen.

2. A compound having the following structure: 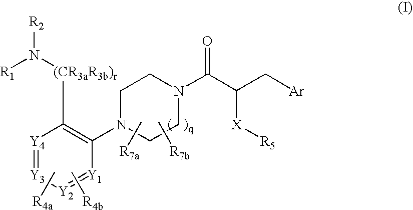
or a stereoisomer, prodrug or pharmaceutically acceptable salt thereof,
wherein:
q is 1 or 2;
r is 1, 2, or 3;
Y₁, Y₂, Y₃ and Y₄ are independently CH or N, with the proviso that no more than two of Y₁, Y₂, Y₃ and Y₄ are N, and with the further proviso that, when two of Y₁, Y₂, Y₃ and Y₄ are N, either Y₁ and Y₃ are N or Y₂ and Y₄ are N;
Ar is phenyl, substituted phenyl, naphthyl, or substituted naphthyl;
X is a bond;
R₁ and R₂ are the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₃ₐ and R_{3b} are, at each occurrence, the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₄ₐ and R_{4b} are optional ring substituents and, when one or both are present, are the same or different and independently hydroxy, alkyl, substituted alkyl, cyano, halogen, alkoxy, or alkylamino;
R₅ is hydrogen, methyl, heterocycle, or substituted heterocycle; and
R₇ₐ and R_{7b} are optional ring substituents and, when one or both are present, are the same or different and independently hydrogen, lower alkyl, or substituted lower alkyl;
with the proviso that when r is 1 then R₁, R₂, R₃ₐ and R_{3b} are not all hydrogen.

3. The compound of claim 2 wherein R₅ is hydrogen.

4. The compound of claim 2 where R₅ is methyl.

5. The compound of claim 2 wherein R₅ is heterocycle or substituted heterocycle.

6. A compound having the following structure: 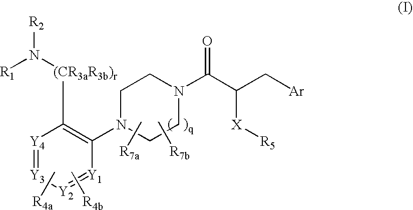
or a stereoisomer, prodrug or pharmaceutically acceptable salt thereof,
wherein:
q is 1 or 2;
r is 1, 2, or 3;
Y₁, Y₂, Y₃ and Y₄ are independently CH or N, with the proviso that no more than two of Y₁, Y₂, Y₃ and Y₄ are N, and with the further proviso that, when two of Y₁, Y₂, Y₃ and Y₄ are N, either Y₁ and Y₃ are N or Y₂ and Y₄ are N;
Ar is phenyl, substituted phenyl, naphthyl, or substituted naphthyl;
X is —S—, —C(═O)O—, —N(R₆ₐ)C(═O)N(R_{6b})O—, or —N(R₆ₐ)C(═O)N(R_{6b})N(R_{6c})—;
R₁ and R₂ are the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₃ₐ and R_{3b} are, at each occurrence, the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₄ₐ and R_{4b} are optional ring substituents and, when one or both are present, are the same or different and independently hydroxy, alkyl, substituted alkyl, cyano, halogen, alkoxy, or alkylamino;
R₅ is hydrogen, alkyl, substituted alkyl, aryl, substituted aryl, heterocycle, or substituted heterocycle;
R₆ₐ, R_{6b} and R_{6c} are, at each occurrence, the same or different and independently hydrogen, alkyl, or substituted alkyl; and
R₇ₐ and R_{7b} are optional ring substituents and, when one or both are present, are the same or different and independently hydrogen, lower alkyl, or substituted lower alkyl;
with the proviso that when r is 1 then R₁, R₂, R₃ₐ and R_{3b} are not all hydrogen.

7. A compound having the following structure: 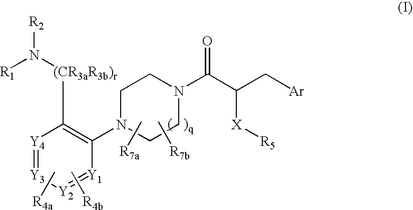
or a stereoisomer, prodrug or pharmaceutically acceptable salt thereof,
wherein:
q is 1 or 2;
r is 1, 2, or 3;
Y₁, Y₂, Y₃ and Y₄ are independently CH or N, with the proviso that no more than two of Y₁, Y₂, Y₃ and Y₄ are N, and with the further proviso that, when two of Y₁, Y₂, Y₃ and Y₄ are N, either Y₁ and Y₃ are N or Y₂ and Y₄ are N;
Ar is phenyl, substituted phenyl, naphthyl, or substituted naphthyl;
X is a —N(R₆ₐ)—, —N(R₆ₐ)C(═O)—, —N(R₆ₐ)S(═O)₂—, —N(R₆ₐ)C(═O)N(R_{6b})—, or —N(R₆ₐ)C(═O)O—;
R₁ and R₂ are the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₃ₐ and R_{3b} are, at each occurrence, the same or different and independently hydrogen, alkyl, substituted alkyl, aryl, substituted aryl, arylalkyl, substituted arylalkyl, heterocycle, substituted heterocycle, heterocyclealkyl, or substituted heterocyclealkyl;
R₄ₐ and R_{4b} are optional ring substituents and, when one or both are present, are the same or different and independently hydroxy, alkyl, substituted alkyl, cyano, halogen, alkoxy, or alkylamino;
R₅ is hydrogen, alkyl, substituted alkyl, aryl, substituted aryl, heterocycle, or substituted heterocycle;
R₆ₐ is alkyl, or substituted alkyl;
R_{6b} is hydrogen, alkyl or substituted alkyl; and
R₇ₐ and R_{7b} are optional ring substituents and, when one or both are present, are the same or different and independently hydrogen, lower alkyl, or substituted lower alkyl;
with the proviso that when r is 1 then R₁, R₂, R₃ₐ and R_{3b} are not all hydrogen.

8. The compound of claim 7 wherein X is —N(R₆ₐ)—.

9. The compound of claim 7 wherein X is —N(R₆ₐ)C(═O)—.

10. The compound of claim 7 wherein X is —N(R₆ₐ)S(═O)₂—.

11. The compound of claim 7 wherein X is —N(R₆ₐ)C(═O)N(R_{6b})—.

12. The compound of claim 7 wherein X is —N(R₆ₐ)C(═O)O—.

13. The compound of any one of claims 1, 2, 6, or 7 wherein Ar is phenyl or substituted phenyl.

14. The compound of any one of claims 1, 2, 6, or 7 wherein Ar is halogen substituted phenyl.

15. The compound of any one of claims 1, 2, 6, or 7 wherein q is 1.

16. The compound of any one of claims 1, 2, 6, or 7 wherein q is 2.

17. The compound of any one of claims 1, 2, 6, or 7 wherein R₁ is alkyl, substituted alkyl, aryl, substituted aryl, arylalkyl, substituted arylalkyl, heterocycle, substituted heterocycle, heterocyclealkyl, or substituted heterocyclealkyl

18. The compound of any one of claims 1, 2, 6, or 7 wherein R₂ is hydrogen.

19. The compound of any one of claims 1, 2, 6, or 7 wherein r is 1.

20. The compound of claim 19 wherein R₃ₐ is hydrogen, alkyl, or substituted alkyl.

21. The compound of claim 19 wherein R_{3b} is hydrogen.

22. The compound of any one of claims 1, 2, 6, or 7 wherein r is 2.

23. The compound of claim 22 wherein R₃ₐ is, at each occurrence, the same or different and independently hydrogen, alkyl, or substituted alkyl.

24. The compound of claim 22 wherein R_{3b} is, at each occurrence, hydrogen.

25. The compound of any one of claims 1, 2, 6, or 7 wherein neither R₄ₐ nor R_{4b} are present.

26. The compound of any one of claims 1, 2, 6, or 7 wherein R₄ₐ is present and is F, Cl, or CF_{3.}

27. The compound of any one of claims 1, 2, 6, or 7 wherein R_{4b} is present and is F or Cl.

28. The compound of claim 1 wherein R₅ is alkyl, substituted alkyl, aryl, or substituted aryl.

29. The compound of any one of claims 1, 2, 6, or 7 wherein neither R₇ₐ nor R_{7b} are present.

30. The compound of any one of claims 1, 2, 6, or 7 wherein one of R₇ₐ or R_{7b} is present.

31. The compound of any one of claims 1, 2, 6, or 7 wherein both R₇ₐ and R_{7b} are present.

32. The compound of any one of claims 1, 2, 6, or 7 wherein each of Y₁, Y₂, Y₃ and Y₄ are CH.

33. The compound of any one of claims 1, 2, 6, or 7 wherein one of Y₁, Y₂, Y₃ and Y₄ is N.

34. The compound of claim 33 wherein Y₁ is N.

35. The compound of claim 33 wherein Y₂ is N.

36. The compound of claim 33 wherein Y₃ is N.

37. The compound of claim 33 wherein Y₄ is N.

38. The compound of any one of claims 1, 2, 6, or 7 wherein two of Y₁, Y₂, Y₃ and Y₄ are N.

39. The compound of claim 38 wherein Y₁ and Y₃ are N.

40. The compound of claim 38 wherein Y₂ and Y₄ are N.

41. The compound of any one of claims 1, 2, 6, or 7 wherein the compound is an agonist of a melanocortin receptor.

42. The compound of claim 41 wherein the melanocortin receptor is melanocortin 3 receptor.

43. The compound of claim 41 wherein the melanocortin receptor is melanocortin 4 receptor.

44. The compound of any one of claims 1, 2, 6, or 7 wherein the compound is an antagonist of a melanocortin receptor.

45. The compound of claim 44 wherein the melanocortin receptor is melanocortin 4 receptor.

46. A composition comprising a compound of any one of claims 1, 2, 6, or 7 in combination with a pharmaceutically acceptable carrier.

47. A method for altering a disorder associated with the activity of a melanocortin receptor, comprising administering to a patient an effective amount of the pharmaceutical composition of claim 46.

48. The method of claim 47 wherein the disorder is an eating disorder.

49. The method of claim 48 wherein the eating disorder is cachexia.

50. The method of claim 47 wherein the disorder is sexual disfunction.

51. The method of claim 50 where the sexual disfunction is erectile disfunction.

52. The method of claim 47 wherein the disorder is a skin disorder.
